(19) United States
(12) Patent Application Publication
(10) Pub. No.: US 2022/0064228 A1
(43) Pub. Date: Mar. 3, 2022

(54) Methods For Increasing The Productivity Of A Filamentous Fungal Cell In The Production Of A Polypeptide

(71) Applicant: Novozymes A/S, Bagsvaerd (DK)

(72) Inventors: Suchindra Maiyuran, Gold River, CA (US); Alfredo Lopez De Leon, Davis, CA (US)

(73) Assignee: Novozymes A/S, Bagsvaerd (DK)

(21) Appl. No.: 17/413,434

(22) PCT Filed: Dec. 12, 2019
(86) PCT No.: PCT/US2019/066046

§ 371 Date: Jun. 11, 2021

Related U.S. Application Data
(60) Provisional application No. 62/778,598, filed on Dec. 12, 2018.

(51) Int. Cl. C07K 14/37; C12N 9/24; C12P 21/00

(52) U.S. Cl. CPC C07K 14/37; C12P 21/00; C12N 9/2402

(57) ABSTRACT

The present invention relates to a mutant of a parent filamentous fungal cell, comprising a coding sequence of a polypeptide of interest under the transcriptional control of a promoter regulated by a transcription factor, wherein the gene encoding the transcription factor is modified in the parent filamentous fungal cell to produce the mutant rendering the mutant deficient in the production of the transcription factor, which increases the productivity of the mutant in the production of the polypeptide of interest, and/or reduces or eliminates the cellulase-negative phenotype in the resulting mutant. The present invention also relates to a method for constructing such a mutant and a method of producing a polypeptide of interest with such a mutant.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to isolated mutant filamentous fungal cells deficient in a transcription factor for the production of polypeptides.

### Description of the Related Art

Filamentous fungi are widely used for producing enzymes and other biologicals for a variety of industrial applications. The productivity of a filamentous fungal cell in the production of a polypeptide of interest is dependent upon several factors, such as carbon source, nitrogen source, secretion, pH, temperature, and dissolved oxygen. In particular, the carbon source can determine which genes for secreted enzymes are induced and/or repressed and their production rates. The carbon source acts through transcription factors and their associated promoters that are either activated or repressed depending on the level of the carbon source.

The expression of cellulases and hemicellulases is generally driven by such promoters. Several transcription factors are known that interact with the promoter regions of cellulase and hemicellulase genes and regulate their expression (Ilmen et al., 1996, Mol. Gen. Genet. 251: 451-460; Aro et al., 2001, J. Biol. Chem. 276: 24309-24314; Zeilinger et al., 2001, Mol. Genet. Genom. 266: 56-63; Aro et al., 2003, Appl. Environ. Microbiol. 69: 56-65; Mach et al., 2003, Appl. Microbiol. Biotechnol. 60: 515-522; Schmoll et al., 2003, Acta Microbiologica et Immunologica Hungarica 50: 125-145; Strieker et al., 2006, Eukaryotic Cell 5: 2128-2137; Strieker et al., 2007, FEBS Letters 581: 3915-3920; Seidl et al., 2008, BMC Genomics 9: 327-341; Strieker et al., 2008, Appl. Microbiol. Biotechnol. 78: 211-220; Kubicek et al., 2009, Biotechnology for Biofuels 2: 19-3; Nakari-Setala et al., 2009, Appl. Environ. Microbiol. 75: 4853-4860).

The development of a cellulase-negative phenotype can arise after prolonged fermentation of filamentous fungi that produce cellulase. There is a need in the art to identify genes encoding transcription factors that can be modified to reduce or eliminate the cellulase-negative phenotype.

The present invention provides improved methods for increasing the productivity of a filamentous fungal cell in the production of a polypeptide of interest in which the activity of a transcription factor has been modified.

### SUMMARY OF THE INVENTION

The present invention relates to an isolated mutant of a parent filamentous fungal cell, comprising a coding sequence of a polypeptide of interest under the transcriptional control of a promoter regulated by one or more transcription factors selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;

wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii).

The present invention also relates to a method of producing a polypeptide of interest, comprising cultivating such a mutant filamentous fungal cell in a medium for production of the polypeptide of interest, and optionally recovering the polypeptide of interest.

The present invention also relates to a method for constructing a mutant of a parent filamentous fungal cell, comprising modifying one or more genes each encoding a transcription factor in the parent filamentous fungal cell to produce the mutant, wherein the parent filamentous fungal cell or the mutant thereof comprises a coding sequence of a polypeptide of interest under the transcriptional control of a promoter regulated by one or more of the transcription factors, wherein the one or more transcription factors are selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;

wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii); and optionally recovering the mutant.

The present invention also relates to an isolated transcription factor, selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

The present invention also relates to an isolated polynucleotide encoding a transcription factor of the present invention; a nucleic acid construct, an expression vector, and a recombinant host cell comprising a polynucleotide encoding a transcription factor of the present invention; and methods of producing such a transcription factor.

### BRIEF DESCRIPTION OF THE FIGURES

FIG. 1 shows a map of plasmid pSMai320.

FIG. 2 shows a map of plasmid pSMai321.

FIG. 3 shows a map of plasmid pSMai322a.

FIG. 4 shows phenotypic analysis of the number of cellulase producing colonies on CMC plates as a function of time for the T. reesei GMer62-1A9 fermentations.

FIG. 5 shows a map of plasmid pBTP01.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd.) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc.).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” or “AA9 lytic polysaccharide monooxygenase” means a polypeptide classified as a lytic polysaccharide monooxygenase (Quinlan et al., 2011, Proc. Natl. Acad. Sci. USA 108: 15079-15084; Phillips et al., 2011, ACS Chem. Biol. 6: 1399-1406; Li et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., and a suitable pH, such as 4-9, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5 L (Novozymes A/S, Bagsværd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can be used in the presence of a soluble activating divalent metal cation according to WO 2008/151043 or WO 2012/122518, e.g., manganese or copper.

The AA9 polypeptide can also be used in the presence of a dioxy compound, a bicyclic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1-4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Catalase: The term “catalase” means a hydrogen-peroxide:hydrogen-peroxide oxidoreductase (E.C. 1.11.1.6 or E.C. 1.11.1.21) that catalyzes the conversion of two hydrogen peroxides to oxygen and two waters.

Catalase activity can be determined by monitoring the degradation of hydrogen peroxide at 240 nm based on the following reaction:

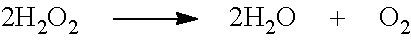

The reaction is conducted in 50 mM phosphate pH 7 at 25° C. with 10.3 mM substrate (H₂O₂). Absorbance is monitored spectrophotometrically within 16-24 seconds, which should correspond to an absorbance reduction from 0.45 to 0.4. One catalase activity unit can be expressed as one μmole of H₂O₂ degraded per minute at pH 7.0 and 25° C.

cDNA: The term “cDNA” means a DNA molecule that can be prepared by reverse transcription from a mature, spliced, mRNA molecule obtained from a eukaryotic or prokaryotic cell. cDNA lacks intron sequences that can be present in the corresponding genomic DNA. The initial, primary RNA transcript is a precursor to mRNA that is processed through a series of steps, including splicing, before appearing as mature spliced mRNA.

cel− phenotype: The term “cel− phenotype” or “cel−” or “cellulase-negative phenotype” means a filamentous fungal cell that cannot produce any cellulase protein when provided with a carbon source containing a cellulase-inducing carbohydrate. For example, filamentous fungal cells exhibiting a cel− phenotype do not produce halos or clearing zones of digested cellulose around filamentous fungal colonies growing on agar medium containing a cellulose substrate. Alternatively, a filamentous fungal cell exhibiting a cel− phenotype does not secrete measurable cellulase protein into culture medium when the filamentous fungal cells are grown in liquid culture medium containing a cellulase-inducing carbohydrate.

cel+ phenotype: The term “cel+ phenotype” or “cel+” or “cellulase-positive phenotype” means a filamentous fungal cell that can produce any cellulase protein when provided with a carbon source containing a cellulase-inducing carbohydrate. For example, filamentous fungal cells exhibiting a cel+ phenotype produce halos or clearing zones of digested cellulose around filamentous fungal colonies growing on agar medium containing a cellulose substrate. Alternatively, a filamentous fungal cell exhibiting a cel+ phenotype does secrete measurable cellulase protein into culture medium when the filamentous fungal cells are grown in liquid culture medium containing a cellulase-inducing carbohydrate. In one aspect, at least 70% of the colonies produce cellulase. In another aspect, at least 75% of the colonies produce cellulase. In another aspect, at least 80% of the colonies produce cellulase. In another aspect, at least 90% of the colonies produce cellulase. In another aspect, at least 95% of the colonies produce cellulase.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teed, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, carboxymethylcellulose, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature, such as 40° C.-80° C., and a suitable pH, such as 4-9, compared to a control hydrolysis without addition of cellulolytic enzyme protein. Typical conditions are 1 ml reactions, washed or unwashed PCS, 5% insoluble solids (dry weight), 50 mM sodium acetate pH 5, 0.1 mM CuCl₂, 50° C., 55° C., or 60° C., 72 hours, sugar analysis by AMINEX® HPX-87H column chromatography (Bio-Rad Laboratories, Inc., Hercules, Calif., USA).

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence can be a genomic DNA, cDNA, synthetic DNA, or a combination thereof.

Control sequences: The term “control sequences” means nucleic acid sequences necessary for expression of a coding sequence for a polypeptide. Each control sequence can be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences can be provided with linkers for the purpose of introducing specific restriction sites facilitating ligation of the control sequences with the coding region of the polynucleotide encoding a polypeptide.

Deficient: The term “deficient” means the gene encoding a transcription factor of the present invention is modified in a parent filamentous fungal cell to produce a mutant rendering the mutant partially deficient (at least 25% less, more preferably at least 50% less, even more preferably at least 75% less, and most preferably at least 95% less transcription factor) or completely deficient (100% less transcription factor) in the production of the transcription factor compared to the parent filamentous fungal cell without the modification of the transcription factor gene when cultivated under identical conditions. The level of a transcription factor produced by a filamentous fungal cell, parent or mutant, can be determined using methods described herein or known in the art.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Fragment: The term “fragment” means a transcription factor having one or more amino acids absent from the amino and/or carboxyl terminus of the transcription factor, wherein the fragment has transcription regulating activity. In one aspect, a fragment contains at least 85%, at least 90%, or at least 95% of the amino acid residues of the full-length transcription factor, e.g., SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Hemicellulolytic enzyme or hemicellulase: The term “hemicellulolytic enzyme” or “hemicellulase” means one or more enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, such as 40° C.-80° C., and a suitable pH, such as 4-9.

Host cell: The term “host cell” means any filamentous fungal cell that is susceptible to transformation, transfection, transduction, or the like with a nucleic acid construct or expression vector comprising a polynucleotide encoding a polypeptide. The term “host cell” encompasses any progeny of a cell that is not identical to the cell due to mutations that occur during replication. In one aspect, the host cell comprises a modification of a transcription factor gene of the present invention.

Increased productivity: The term “increased productivity” and variations thereof mean an increase of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% in the production of a polypeptide of interest by a mutant filamentous fungal cell with a modification of a transcription factor gene of the present invention when cultivated under the same conditions of medium composition, temperature, pH, cell density, dissolved oxygen, and time as the parent filamentous fungal cell without the modification. In one aspect, the productivity of the mutant filamentous fungal cell with a modification of a transcription factor gene is increased 1%, 2%, 3%, 4%, 5% 6%, 7%, 8%, 9%, 10%, 11%, 12%, 13%, 14%, 15%, 16%, 17%, 18%, 19%, or 20% in the production of the polypeptide of interest compared to the parent filamentous fungal cell without the modification.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, or peptide, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Laccase: The term “laccase” means a benzenediol:oxygen oxidoreductase (E.C. 1.10.3.2) that catalyzes the following reaction: 1,2- or 1,4-benzenediol+O₂=1,2- or 1,4-benzosemiquinone+2H₂O.

Laccase activity can be determined by the oxidation of syringaldazine (4,4′-[azinobis(methanylylidene)]bis(2,6-dimethoxyphenol)) to the corresponding quinone 4,4′-[azobis(methanylylidene)]bis(2,6-dimethoxycyclohexa-2,5-dien-1-one) by laccase. The reaction (shown below) is detected by an increase in absorbance at 530 nm.

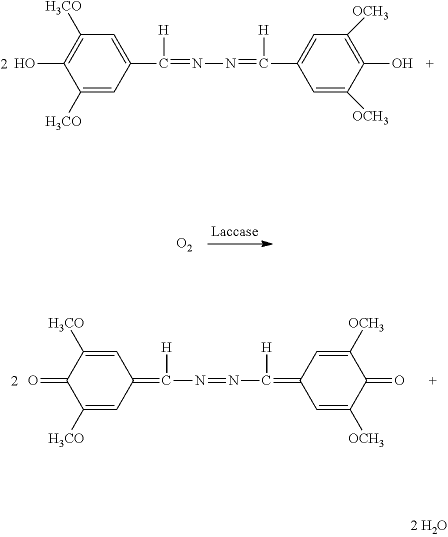 [SMILES: COc1cc(C=NN=Cc2cc(OC)c(O)c(OC)c2)cc(OC)c1O ; "O2" ; COC1=CC(=CN=NC=C2C=C(OC)C(=O)C(OC)=C2)C=C(OC)C1=O]

The reaction is conducted in 23 mM MES pH 5.5 at 30° C. with 19 μM substrate (syringaldazine) and 1 g/L polyethylene glycol (PEG) 6000 and the change in absorbance is measured at 530 nm every 15 seconds up to 90 seconds. One laccase unit is the amount of enzyme that catalyzes the conversion of 1 μmole syringaldazine per minute under the specified analytical conditions.

Lysozyme: The term “lysozyme” means a peptidoglycan N-acetylmuramoylhydrolase (E.C. 3.2.1.17) that catalyzes the hydrolysis of (1->4)-beta-linkages between N-acetylmuramic acid and N-acetyl-D-glucosamine residues in a peptidoglycan and between N-acetyl-D-glucosamine residues in chitodextrins. Lysozyme activity can be determined according to the assay described in Example 11.

Peroxidase: The term “peroxidase” means an enzyme that converts a peroxide, e.g., hydrogen peroxide, to a less oxidative species, e.g., water. It is understood herein that a peroxidase encompasses a peroxide-decomposing enzyme. The term “peroxide-decomposing enzyme” is defined herein as a donor:peroxide oxidoreductase (E.C. number 1.11.1.x, wherein x=1-3, 5, 7-19, or 21) that catalyzes the reaction reduced substrate (2e⁻)+ROOR′→oxidized substrate+ROH+R′OH; such as horseradish peroxidase that catalyzes the reaction phenol+H₂O₂→quinone+H₂O, and catalase that catalyzes the reaction H₂O₂+H₂O₂→O₂+2H₂O. In addition to hydrogen peroxide, other peroxides may also be decomposed by these enzymes.

Peroxidase activity can be determined by measuring the oxidation of 2,2′-azino-bis(3-ethylbenzthiazoline-6-sulfonic acid) (ABTS) by a peroxidase in the presence of hydrogen peroxide as shown below. The reaction product ABTSₒₓ forms a blue-green color which can be quantified at 418 nm.

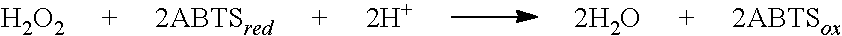

The reaction is conducted in 0.1 M phosphate pH 7 at 30° C. with 1.67 mM substrate (ABTS), 1.5 g/L TRITON® X-405, 0.88 mM hydrogen peroxide, and approximately 0.040 unit enzyme per ml and the change in absorbance is measured at 418 nm from 15 seconds up to 60 seconds. One peroxidase unit can be expressed as the amount of enzyme required to catalyze the conversion of 1 μmole of hydrogen peroxide per minute under the specified analytical conditions.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are a gap open penalty of 10, a gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Transcription factor: The term “transcription factor” means a polypeptide having transcription regulating activity.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

The present invention relates to an isolated mutant of a parent filamentous fungal cell, comprising a coding sequence of a polypeptide of interest under the transcriptional control of a promoter regulated by one or more transcription factors selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;

wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii).

The present invention also relates to a method for constructing a mutant of a parent filamentous fungal cell, comprising modifying one or more genes each encoding a transcription factor in the parent filamentous fungal cell to produce the mutant, wherein the parent filamentous fungal cell or the mutant thereof comprises a coding sequence of a polypeptide of interest under the transcriptional control of a promoter regulated by one or more of the transcription factors, wherein the one or more transcription factors are selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;

wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii); and optionally recovering the mutant.

An advantage of the present invention is that modification of one or more genes each encoding a transcription factor of the present invention in a parent filamentous fungal cell can reduce or eliminate the cellulase-negative phenotype in the resulting mutant, which can increase the productivity of the mutant in the production of a polypeptide of interest when cultivated under the same conditions as the parent filamentous fungal cell without the modification. In one aspect, the modification of the one or more transcription factor genes increases the productivity of the mutant in the production of the polypeptide of interest when cultivated under the same conditions as the parent filamentous fungal cell without the modification of the one or more transcription factor genes. In another aspect, the modification of the one or more transcription factor genes reduces or eliminates the cellulase-negative phenotype in the resulting mutant compared to the parent filamentous fungal cell without the modification of the one or more transcription factor genes. In another aspect, the modification of the one or more transcription factor genes (i) increases the productivity of the mutant in the production of the polypeptide of interest when cultivated under the same conditions as the parent filamentous fungal cell without the modification of the one or more transcription factor genes, and (ii) reduces or eliminates the cellulase-negative phenotype in the resulting mutant compared to the parent filamentous fungal cell without the modification of the one or more transcription factor genes.

### Transcription Factors

In one aspect, the transcription factor has a sequence identity of at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

In one embodiment, the transcription factor differs by up to 10 amino acids, e.g., 1, 2, 3, 4, 5, 6, 7, 8, 9, or 10, from SEQ ID NO: 2. In another embodiment, the transcription factor differs by up to 10 amino acids, e.g., 1, 2, 3, 4, 5, 6, 7, 8, 9, or 10, from SEQ ID NO: 4. In another embodiment, the transcription factor differs by up to 10 amino acids, e.g., 1, 2, 3, 4, 5, 6, 7, 8, 9, or 10, from SEQ ID NO: 6. In another embodiment, the transcription factor differs by up to 10 amino acids, e.g., 1, 2, 3, 4, 5, 6, 7, 8, 9, or 10, from SEQ ID NO: 8. In another embodiment, the transcription factor differs by up to 10 amino acids, e.g., 1, 2, 3, 4, 5, 6, 7, 8, 9, or 10, from SEQ ID NO: 10. In another embodiment, the transcription factor differs by up to 10 amino acids, e.g., 1, 2, 3, 4, 5, 6, 7, 8, 9, or 10, from SEQ ID NO: 12. In another embodiment, the transcription factor differs by up to 10 amino acids, e.g., 1, 2, 3, 4, 5, 6, 7, 8, 9, or 10, from SEQ ID NO: 14. In another embodiment, the transcription factor differs by up to 10 amino acids, e.g., 1, 2, 3, 4, 5, 6, 7, 8, 9, or 10, from SEQ ID NO: 16. In another embodiment, the transcription factor differs by up to 10 amino acids, e.g., 1, 2, 3, 4, 5, 6, 7, 8, 9, or 10, from SEQ ID NO: 18. In another embodiment, the transcription factor differs by up to 10 amino acids, e.g., 1, 2, 3, 4, 5, 6, 7, 8, 9, or 10, from SEQ ID NO: 20. In another embodiment, the transcription factor differs by up to 10 amino acids, e.g., 1, 2, 3, 4, 5, 6, 7, 8, 9, or 10, from SEQ ID NO: 22. In another embodiment, the transcription factor differs by up to 10 amino acids, e.g., 1, 2, 3, 4, 5, 6, 7, 8, 9, or 10, from SEQ ID NO: 24. In another embodiment, the transcription factor differs by up to 10 amino acids, e.g., 1, 2, 3, 4, 5, 6, 7, 8, 9, or 10, from SEQ ID NO: 26. In another embodiment, the transcription factor differs by up to 10 amino acids, e.g., 1, 2, 3, 4, 5, 6, 7, 8, 9, or 10, from SEQ ID NO: 28. In another embodiment, the transcription factor differs by up to 10 amino acids, e.g., 1, 2, 3, 4, 5, 6, 7, 8, 9, or 10, from SEQ ID NO: 30. In another embodiment, the transcription factor differs by up to 10 amino acids, e.g., 1, 2, 3, 4, 5, 6, 7, 8, 9, or 10, from SEQ ID NO: 32. In another embodiment, the transcription factor differs by up to 10 amino acids, e.g., 1, 2, 3, 4, 5, 6, 7, 8, 9, or 10, from SEQ ID NO: 34. In another embodiment, the transcription factor differs by up to 10 amino acids, e.g., 1, 2, 3, 4, 5, 6, 7, 8, 9, or 10, from SEQ ID NO: 36. In another embodiment, the transcription factor differs by up to 10 amino acids, e.g., 1, 2, 3, 4, 5, 6, 7, 8, 9, or 10, from SEQ ID NO: 38. In another embodiment, the transcription factor differs by up to 10 amino acids, e.g., 1, 2, 3, 4, 5, 6, 7, 8, 9, or 10, from SEQ ID NO: 40. In another embodiment, the transcription factor differs by up to 10 amino acids, e.g., 1, 2, 3, 4, 5, 6, 7, 8, 9, or 10, from SEQ ID NO: 42. In another embodiment, the transcription factor differs by up to 10 amino acids, e.g., 1, 2, 3, 4, 5, 6, 7, 8, 9, or 10, from SEQ ID NO: 44. In another embodiment, the transcription factor differs by up to 10 amino acids, e.g., 1, 2, 3, 4, 5, 6, 7, 8, 9, or 10, from SEQ ID NO: 46. In another embodiment, the transcription factor differs by up to 10 amino acids, e.g., 1, 2, 3, 4, 5, 6, 7, 8, 9, or 10, from SEQ ID NO: 48. In another embodiment, the transcription factor differs by up to 10 amino acids, e.g., 1, 2, 3, 4, 5, 6, 7, 8, 9, or 10, from SEQ ID NO: 50. In another embodiment, the transcription factor differs by up to 10 amino acids, e.g., 1, 2, 3, 4, 5, 6, 7, 8, 9, or 10, from SEQ ID NO: 52. In another embodiment, the transcription factor differs by up to 10 amino acids, e.g., 1, 2, 3, 4, 5, 6, 7, 8, 9, or 10, from SEQ ID NO: 54. In another embodiment, the transcription factor differs by up to 10 amino acids, e.g., 1, 2, 3, 4, 5, 6, 7, 8, 9, or 10, from SEQ ID NO: 56. In another embodiment, the transcription factor differs by up to 10 amino acids, e.g., 1, 2, 3, 4, 5, 6, 7, 8, 9, or 10, from SEQ ID NO: 124.

In another embodiment, the transcription factor comprises the amino acid sequence of SEQ ID NO: 2 or an allelic variant thereof. In another embodiment, the transcription factor comprises the amino acid sequence of SEQ ID NO: 2. In another embodiment, the transcription factor consists of the amino acid sequence of SEQ ID NO: 2. In another embodiment, the transcription factor comprises the amino acid sequence of SEQ ID NO: 4 or an allelic variant thereof. In another embodiment, the transcription factor comprises the amino acid sequence of SEQ ID NO: 4. In another embodiment, the transcription factor consists of the amino acid sequence of SEQ ID NO: 4. In another embodiment, the transcription factor comprises the amino acid sequence of SEQ ID NO: 6 or an allelic variant thereof. In another embodiment, the transcription factor comprises the amino acid sequence of SEQ ID NO: 6. In another embodiment, the transcription factor consists of the amino acid sequence of SEQ ID NO: 6. In another embodiment, the transcription factor comprises the amino acid sequence of SEQ ID NO: 8 or an allelic variant thereof. In another embodiment, the transcription factor comprises the amino acid sequence of SEQ ID NO: 8. In another embodiment, the transcription factor consists of the amino acid sequence of SEQ ID NO: 8. In another embodiment, the transcription factor comprises the amino acid sequence of SEQ ID NO: 10 or an allelic variant thereof. In another embodiment, the transcription factor comprises the amino acid sequence of SEQ ID NO: 10. In another embodiment, the transcription factor consists of the amino acid sequence of SEQ ID NO: 10. In another embodiment, the transcription factor comprises the amino acid sequence of SEQ ID NO: 12 or an allelic variant thereof. In another embodiment, the transcription factor comprises the amino acid sequence of SEQ ID NO: 12. In another embodiment, the transcription factor consists of the amino acid sequence of SEQ ID NO: 12. In another embodiment, the transcription factor comprises the amino acid sequence of SEQ ID NO: 14 or an allelic variant thereof. In another embodiment, the transcription factor comprises the amino acid sequence of SEQ ID NO: 14. In another embodiment, the transcription factor consists of the amino acid sequence of SEQ ID NO: 14. In another embodiment, the transcription factor comprises the amino acid sequence of SEQ ID NO: 16 or an allelic variant thereof. In another embodiment, the transcription factor comprises the amino acid sequence of SEQ ID NO: 16. In another embodiment, the transcription factor consists of the amino acid sequence of SEQ ID NO: 16. In another embodiment, the transcription factor comprises the amino acid sequence of SEQ ID NO: 18 or an allelic variant thereof. In another embodiment, the transcription factor comprises the amino acid sequence of SEQ ID NO: 18. In another embodiment, the transcription factor consists of the amino acid sequence of SEQ ID NO: 18. In another embodiment, the transcription factor comprises the amino acid sequence of SEQ ID NO: 20 or an allelic variant thereof. In another embodiment, the transcription factor comprises the amino acid sequence of SEQ ID NO: 20. In another embodiment, the transcription factor consists of the amino acid sequence of SEQ ID NO: 20. In another embodiment, the transcription factor comprises the amino acid sequence of SEQ ID NO: 22 or an allelic variant thereof. In another embodiment, the transcription factor comprises the amino acid sequence of SEQ ID NO: 22. In another embodiment, the transcription factor consists of the amino acid sequence of SEQ ID NO: 22. In another embodiment, the transcription factor comprises the amino acid sequence of SEQ ID NO: 24 or an allelic variant thereof. In another embodiment, the transcription factor comprises the amino acid sequence of SEQ ID NO: 24. In another embodiment, the transcription factor consists of the amino acid sequence of SEQ ID NO: 24. In another embodiment, the transcription factor comprises the amino acid sequence of SEQ ID NO: 26 or an allelic variant thereof. In another embodiment, the transcription factor comprises the amino acid sequence of SEQ ID NO: 26. In another embodiment, the transcription factor consists of the amino acid sequence of SEQ ID NO: 26. In another embodiment, the transcription factor comprises the amino acid sequence of SEQ ID NO: 28 or an allelic variant thereof. In another embodiment, the transcription factor comprises the amino acid sequence of SEQ ID NO: 28. In another embodiment, the transcription factor consists of the amino acid sequence of SEQ ID NO: 28. In another embodiment, the transcription factor comprises the amino acid sequence of SEQ ID NO: 30 or an allelic variant thereof. In another embodiment, the transcription factor comprises the amino acid sequence of SEQ ID NO: 30. In another embodiment, the transcription factor consists of the amino acid sequence of SEQ ID NO: 30. In another embodiment, the transcription factor comprises the amino acid sequence of SEQ ID NO: 32 or an allelic variant thereof. In another embodiment, the transcription factor comprises the amino acid sequence of SEQ ID NO: 32. In another embodiment, the transcription factor consists of the amino acid sequence of SEQ ID NO: 32. In another embodiment, the transcription factor comprises the amino acid sequence of SEQ ID NO: 34 or an allelic variant thereof. In another embodiment, the transcription factor comprises the amino acid sequence of SEQ ID NO: 34. In another embodiment, the transcription factor consists of the amino acid sequence of SEQ ID NO: 34. In another embodiment, the transcription factor comprises the amino acid sequence of SEQ ID NO: 36 or an allelic variant thereof. In another embodiment, the transcription factor comprises the amino acid sequence of SEQ ID NO: 36. In another embodiment, the transcription factor consists of the amino acid sequence of SEQ ID NO: 36. In another embodiment, the transcription factor comprises the amino acid sequence of SEQ ID NO: 38 or an allelic variant thereof. In another embodiment, the transcription factor comprises the amino acid sequence of SEQ ID NO: 38. In another embodiment, the transcription factor consists of the amino acid sequence of SEQ ID NO: 38. In another embodiment, the transcription factor comprises the amino acid sequence of SEQ ID NO: 40 or an allelic variant thereof. In another embodiment, the transcription factor comprises the amino acid sequence of SEQ ID NO: 40. In another embodiment, the transcription factor consists of the amino acid sequence of SEQ ID NO: 40. In another embodiment, the transcription factor comprises the amino acid sequence of SEQ ID NO: 42 or an allelic variant thereof. In another embodiment, the transcription factor comprises the amino acid sequence of SEQ ID NO: 42. In another embodiment, the transcription factor consists of the amino acid sequence of SEQ ID NO: 42. In another embodiment, the transcription factor comprises the amino acid sequence of SEQ ID NO: 44 or an allelic variant thereof. In another embodiment, the transcription factor comprises the amino acid sequence of SEQ ID NO: 44. In another embodiment, the transcription factor consists of the amino acid sequence of SEQ ID NO: 44. In another embodiment, the transcription factor comprises the amino acid sequence of SEQ ID NO: 46 or an allelic variant thereof. In another embodiment, the transcription factor comprises the amino acid sequence of SEQ ID NO: 46. In another embodiment, the transcription factor consists of the amino acid sequence of SEQ ID NO: 46. In another embodiment, the transcription factor comprises the amino acid sequence of SEQ ID NO: 48 or an allelic variant thereof. In another embodiment, the transcription factor comprises the amino acid sequence of SEQ ID NO: 48. In another embodiment, the transcription factor consists of the amino acid sequence of SEQ ID NO: 48. In another embodiment, the transcription factor comprises the amino acid sequence of SEQ ID NO: 50 or an allelic variant thereof. In another embodiment, the transcription factor comprises the amino acid sequence of SEQ ID NO: 50. In another embodiment, the transcription factor consists of the amino acid sequence of SEQ ID NO: 50. In another embodiment, the transcription factor comprises the amino acid sequence of SEQ ID NO: 52 or an allelic variant thereof. In another embodiment, the transcription factor comprises the amino acid sequence of SEQ ID NO: 52. In another embodiment, the transcription factor consists of the amino acid sequence of SEQ ID NO: 52. In another embodiment, the transcription factor comprises the amino acid sequence of SEQ ID NO: 54 or an allelic variant thereof. In another embodiment, the transcription factor comprises the amino acid sequence of SEQ ID NO: 54. In another embodiment, the transcription factor consists of the amino acid sequence of SEQ ID NO: 54. In another embodiment, the transcription factor comprises the amino acid sequence of SEQ ID NO: 56 or an allelic variant thereof. In another embodiment, the transcription factor comprises the amino acid sequence of SEQ ID NO: 56. In another embodiment, the transcription factor consists of the amino acid sequence of SEQ ID NO: 56. In another embodiment, the transcription factor comprises the amino acid sequence of SEQ ID NO: 124 or an allelic variant thereof. In another embodiment, the transcription factor comprises the amino acid sequence of SEQ ID NO: 124. In another embodiment, the transcription factor consists of the amino acid sequence of SEQ ID NO: 124.

In another aspect, the transcription factor is encoded by a polynucleotide having a sequence identity of at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123, or the cDNA sequence thereof.

In one embodiment, the transcription factor is encoded by a polynucleotide comprising SEQ ID NO: 1. In another embodiment, the transcription factor is encoded by a polynucleotide consisting of SEQ ID NO: 1. In another embodiment, the transcription factor is encoded by a polynucleotide comprising SEQ ID NO: 3. In another embodiment, the transcription factor is encoded by a polynucleotide consisting of SEQ ID NO: 3. In another embodiment, the transcription factor is encoded by a polynucleotide comprising SEQ ID NO: 5. In another embodiment, the transcription factor is encoded by a polynucleotide consisting of SEQ ID NO: 5. In another embodiment, the transcription factor is encoded by a polynucleotide comprising SEQ ID NO: 7. In another embodiment, the transcription factor is encoded by a polynucleotide consisting of SEQ ID NO: 7. In another embodiment, the transcription factor is encoded by a polynucleotide comprising SEQ ID NO: 9. In another embodiment, the transcription factor is encoded by a polynucleotide consisting of SEQ ID NO: 9. In another embodiment, the transcription factor is encoded by a polynucleotide comprising SEQ ID NO: 11. In another embodiment, the transcription factor is encoded by a polynucleotide consisting of SEQ ID NO: 11. In another embodiment, the transcription factor is encoded by a polynucleotide comprising of SEQ ID NO: 13. In another embodiment, the transcription factor is encoded by a polynucleotide consisting of SEQ ID NO: 13. In another embodiment, the transcription factor is encoded by a polynucleotide comprising SEQ ID NO: 15. In another embodiment, the transcription factor is encoded by a polynucleotide consisting of SEQ ID NO: 15. In another embodiment, the transcription factor is encoded by a polynucleotide comprising SEQ ID NO: 17. In another embodiment, the transcription factor is encoded by a polynucleotide consisting of SEQ ID NO: 17. In another embodiment, the transcription factor is encoded by a polynucleotide comprising SEQ ID NO: 19. In another embodiment, the transcription factor is encoded by a polynucleotide consisting of SEQ ID NO: 19. In another embodiment, the transcription factor is encoded by a polynucleotide comprising SEQ ID NO: 21. In another embodiment, the transcription factor is encoded by a polynucleotide consisting of SEQ ID NO: 21. In another embodiment, the transcription factor is encoded by a polynucleotide comprising SEQ ID NO: 23. In another embodiment, the transcription factor is encoded by a polynucleotide consisting of SEQ ID NO: 23. In another embodiment, the transcription factor is encoded by a polynucleotide comprising SEQ ID NO: 25. In another embodiment, the transcription factor is encoded by a polynucleotide consisting of SEQ ID NO: 25. In another embodiment, the transcription factor is encoded by a polynucleotide comprising SEQ ID NO: 27. In another embodiment, the transcription factor is encoded by a polynucleotide consisting of SEQ ID NO: 27. In another embodiment, the transcription factor is encoded by a polynucleotide comprising SEQ ID NO: 29. In another embodiment, the transcription factor is encoded by a polynucleotide consisting of SEQ ID NO: 29. In another embodiment, the transcription factor is encoded by a polynucleotide comprising SEQ ID NO: 31. In another embodiment, the transcription factor is encoded by a polynucleotide consisting of SEQ ID NO: 31. In another embodiment, the transcription factor is encoded by a polynucleotide comprising SEQ ID NO: 33. In another embodiment, the transcription factor is encoded by a polynucleotide consisting of SEQ ID NO: 33. In another embodiment, the transcription factor is encoded by a polynucleotide comprising SEQ ID NO: 35. In another embodiment, the transcription factor is encoded by a polynucleotide consisting of SEQ ID NO: 35. In another embodiment, the transcription factor is encoded by a polynucleotide comprising SEQ ID NO: 37. In another embodiment, the transcription factor is encoded by a polynucleotide consisting of SEQ ID NO: 37. In another embodiment, the transcription factor is encoded by a polynucleotide comprising SEQ ID NO: 39. In another embodiment, the transcription factor is encoded by a polynucleotide consisting of SEQ ID NO: 39. In another embodiment, the transcription factor is encoded by a polynucleotide comprising SEQ ID NO: 41. In another embodiment, the transcription factor is encoded by a polynucleotide consisting of SEQ ID NO: 41. In another embodiment, the transcription factor is encoded by a polynucleotide comprising SEQ ID NO: 43. In another embodiment, the transcription factor is encoded by a polynucleotide consisting of SEQ ID NO: 43. In another embodiment, the transcription factor is encoded by a polynucleotide comprising SEQ ID NO: 45. In another embodiment, the transcription factor is encoded by a polynucleotide consisting of SEQ ID NO: 45. In another embodiment, the transcription factor is encoded by a polynucleotide comprising SEQ ID NO: 47. In another embodiment, the transcription factor is encoded by a polynucleotide consisting of SEQ ID NO: 47. In another embodiment, the transcription factor is encoded by a polynucleotide comprising SEQ ID NO: 49. In another embodiment, the transcription factor is encoded by a polynucleotide consisting of SEQ ID NO: 49. In another embodiment, the transcription factor is encoded by a polynucleotide comprising SEQ ID NO: 51. In another embodiment, the transcription factor is encoded by a polynucleotide consisting of SEQ ID NO: 51. In another embodiment, the transcription factor is encoded by a polynucleotide comprising SEQ ID NO: 53. In another embodiment, the transcription factor is encoded by a polynucleotide consisting of SEQ ID NO: 53. In another embodiment, the transcription factor is encoded by a polynucleotide comprising SEQ ID NO: 55. In another embodiment, the transcription factor is encoded by a polynucleotide consisting of SEQ ID NO: 55. In another embodiment, the transcription factor is encoded by a polynucleotide comprising SEQ ID NO: 123. In another embodiment, the transcription factor is encoded by a polynucleotide consisting of SEQ ID NO: 123.

In another aspect, the transcription factor is encoded by a polynucleotide that hybridizes under very low stringency conditions, low stringency conditions, medium stringency conditions, medium-high stringency conditions, high stringency conditions, or very high stringency conditions with (i) SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123, (ii) the cDNA sequence thereof, or (iii) the full-length complement of (i) or (ii) (Sambrook et al., 1989, Molecular Cloning, A Laboratory Manual, 2d edition, Cold Spring Harbor, N.Y.).

The polynucleotide of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123, or a subsequence thereof, as well as the polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124, or a fragment thereof, can be used to design nucleic acid probes to identify and clone DNA encoding transcription factors from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains can be screened for DNA that hybridizes with the probes described above and encodes a transcription factor. Genomic or other DNA from such other strains can be separated by agarose or polyacrylamide gel electrophoresis, or other separation techniques. DNA from the libraries or the separated DNA can be transferred to and immobilized on nitrocellulose or other suitable carrier material. In order to identify a clone or DNA that hybridizes with SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123, or a subsequence thereof, the carrier material is used in a Southern blot.

For purposes of the present invention, hybridization indicates that the polynucleotides hybridize to a labeled nucleic acid probe corresponding to (i) SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; (ii) the cDNA sequence thereof; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one embodiment, the nucleic acid probe is SEQ ID NO: 1 or the cDNA sequence thereof. In another embodiment, the nucleic acid probe is SEQ ID NO: 3 or the cDNA sequence thereof. In another embodiment, the nucleic acid probe is SEQ ID NO: 5 or the cDNA sequence thereof. In another embodiment, the nucleic acid probe is SEQ ID NO: 7 or the cDNA sequence thereof. In another embodiment, the nucleic acid probe is SEQ ID NO: 9 or the cDNA sequence thereof. In another embodiment, the nucleic acid probe is SEQ ID NO: 11 or the cDNA sequence thereof. In another embodiment, the nucleic acid probe is SEQ ID NO: 13 or the cDNA sequence thereof. In another embodiment, the nucleic acid probe is SEQ ID NO: 15 or the cDNA sequence thereof. In another embodiment, the nucleic acid probe is SEQ ID NO: 17 or the cDNA sequence thereof. In another embodiment, the nucleic acid probe is SEQ ID NO: 19 or the cDNA sequence thereof. In another embodiment, the nucleic acid probe is SEQ ID NO: 21 or the cDNA sequence thereof. In another embodiment, the nucleic acid probe is SEQ ID NO: 23 or the cDNA sequence thereof. In another embodiment, the nucleic acid probe is SEQ ID NO: 25 or the cDNA sequence thereof. In another embodiment, the nucleic acid probe is SEQ ID NO: 27 or the cDNA sequence thereof. In another embodiment, the nucleic acid probe is SEQ ID NO: 29 or the cDNA sequence thereof. In another embodiment, the nucleic acid probe is SEQ ID NO: 31 or the cDNA sequence thereof. In another embodiment, the nucleic acid probe is SEQ ID NO: 33 or the cDNA sequence thereof. In another embodiment, the nucleic acid probe is SEQ ID NO: 35 or the cDNA sequence thereof. In another embodiment, the nucleic acid probe is SEQ ID NO: 37 or the cDNA sequence thereof. In another embodiment, the nucleic acid probe is SEQ ID NO: 39 or the cDNA sequence thereof. In another embodiment, the nucleic acid probe is SEQ ID NO: 41 or the cDNA sequence thereof. In another embodiment, the nucleic acid probe is SEQ ID NO: 43 or the cDNA sequence thereof. In another embodiment, the nucleic acid probe is SEQ ID NO: 45 or the cDNA sequence thereof. In another embodiment, the nucleic acid probe is SEQ ID NO: 47 or the cDNA sequence thereof. In another embodiment, the nucleic acid probe is SEQ ID NO: 49 or the cDNA sequence thereof. In another embodiment, the nucleic acid probe is SEQ ID NO: 51 or the cDNA sequence thereof. In another embodiment, the nucleic acid probe is SEQ ID NO: 53 or the cDNA sequence thereof. In another embodiment, the nucleic acid probe is SEQ ID NO: 55 or the cDNA sequence thereof. In another embodiment, the nucleic acid probe is SEQ ID NO: 123 or the cDNA sequence thereof.

In another embodiment, the nucleic acid probe is a polynucleotide that encodes the transcription factor of SEQ ID NO: 2 or a fragment thereof. In another embodiment, the nucleic acid probe is a polynucleotide that encodes the transcription factor of SEQ ID NO: 4 or a fragment thereof. In another embodiment, the nucleic acid probe is a polynucleotide that encodes the transcription factor of SEQ ID NO: 6 or a fragment thereof. In another embodiment, the nucleic acid probe is a polynucleotide that encodes the transcription factor of SEQ ID NO: 8 or a fragment thereof. In another embodiment, the nucleic acid probe is a polynucleotide that encodes the transcription factor of SEQ ID NO: 10 or a fragment thereof. In another embodiment, the nucleic acid probe is a polynucleotide that encodes the transcription factor of SEQ ID NO: 12 or a fragment thereof. In another embodiment, the nucleic acid probe is a polynucleotide that encodes the transcription factor of SEQ ID NO: 14 or a fragment thereof. In another embodiment, the nucleic acid probe is a polynucleotide that encodes the transcription factor of SEQ ID NO: 16 or a fragment thereof. In another embodiment, the nucleic acid probe is a polynucleotide that encodes the transcription factor of SEQ ID NO: 18 or a fragment thereof. In another embodiment, the nucleic acid probe is a polynucleotide that encodes the transcription factor of SEQ ID NO: 20 or a fragment thereof. In another embodiment, the nucleic acid probe is a polynucleotide that encodes the transcription factor of SEQ ID NO: 22 or a fragment thereof. In another embodiment, the nucleic acid probe is a polynucleotide that encodes the transcription factor of SEQ ID NO: 24 or a fragment thereof. In another embodiment, the nucleic acid probe is a polynucleotide that encodes the transcription factor of SEQ ID NO: 26 or a fragment thereof. In another embodiment, the nucleic acid probe is a polynucleotide that encodes the transcription factor of SEQ ID NO: 28 or a fragment thereof. In another embodiment, the nucleic acid probe is a polynucleotide that encodes the transcription factor of SEQ ID NO: 30 or a fragment thereof. In another embodiment, the nucleic acid probe is a polynucleotide that encodes the transcription factor of SEQ ID NO: 32 or a fragment thereof. In another embodiment, the nucleic acid probe is a polynucleotide that encodes the transcription factor of SEQ ID NO: 34 or a fragment thereof. In another embodiment, the nucleic acid probe is a polynucleotide that encodes the transcription factor of SEQ ID NO: 36 or a fragment thereof. In another embodiment, the nucleic acid probe is a polynucleotide that encodes the transcription factor of SEQ ID NO: 38 or a fragment thereof. In another embodiment, the nucleic acid probe is a polynucleotide that encodes the transcription factor of SEQ ID NO: 40 or a fragment thereof. In another embodiment, the nucleic acid probe is a polynucleotide that encodes the transcription factor of SEQ ID NO: 42 or a fragment thereof. In another embodiment, the nucleic acid probe is a polynucleotide that encodes the transcription factor of SEQ ID NO: 44 or a fragment thereof. In another embodiment, the nucleic acid probe is a polynucleotide that encodes the transcription factor of SEQ ID NO: 46 or a fragment thereof. In another embodiment, the nucleic acid probe is a polynucleotide that encodes the transcription factor of SEQ ID NO: 48 or a fragment thereof. In another embodiment, the nucleic acid probe is a polynucleotide that encodes the transcription factor of SEQ ID NO: 50 or a fragment thereof. In another embodiment, the nucleic acid probe is a polynucleotide that encodes the transcription factor of SEQ ID NO: 52 or a fragment thereof. In another embodiment, the nucleic acid probe is a polynucleotide that encodes the transcription factor of SEQ ID NO: 54 or a fragment thereof. In another embodiment, the nucleic acid probe is a polynucleotide that encodes the transcription factor of SEQ ID NO: 56 or a fragment thereof. In another embodiment, the nucleic acid probe is a polynucleotide that encodes the transcription factor of SEQ ID NO: 124 or a fragment thereof.

### Parent Filamentous Fungal Cells

In the present invention, the parent filamentous fungal cell can be any filamentous fungal cell. The filamentous fungal cell can be a wild-type cell or a mutant thereof.

“Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

In one aspect, the parent filamentous fungal cell is an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimasfix, Neurospora, Paecilomyces, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria cell.

In an embodiment, the parent filamentous fungal cell is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Talaromyces emersonii, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride cell.

In another embodiment, the parent filamentous fungal cell is a Myceliophthora thermophila cell.

In another embodiment, the parent filamentous fungal cell is a Talaromyces emersonii cell.

In another embodiment, the parent filamentous fungal cell is a Trichoderma harzianum cell.

In another embodiment, the parent filamentous fungal cell is a Trichoderma koningii cell.

In another embodiment, the parent filamentous fungal cell is a Trichoderma longibrachiatum cell.

In another embodiment, the parent filamentous fungal cell is a Trichoderma reesei cell.

In another embodiment, the parent filamentous fungal cell is a Trichoderma viride cell.

In a preferred embodiment, the parent Trichoderma reesei cell is Trichoderma reesei Rut-C30.

In another preferred embodiment, the parent Trichoderma reesei cell is a mutant of Trichoderma reesei.

In another preferred embodiment, the parent Trichoderma reesei cell is a morphological mutant of Trichoderma reesei (see WO 97/26330).

In another preferred embodiment, the parent Trichoderma reesei cell is a protease-deficient mutant of Trichoderma reesei (see WO 2011/075677).

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

A mutant filamentous fungal cell deficient in a transcription factor of the invention can be constructed by reducing or eliminating expression of the gene encoding the transcription factor using methods well known in the art. A portion of the gene can be modified such as the coding region or a control sequence required for expression of the coding region. Such a control sequence of the gene can be a promoter sequence or a functional part thereof, i.e., a part that is sufficient for affecting expression of the gene. For example, a promoter sequence can be inactivated resulting in no expression or a weaker promoter can be substituted for the native promoter sequence to reduce expression of the coding sequence.

The mutant filamentous fungal cell can be constructed by gene deletion techniques to eliminate or reduce expression of the gene. Gene deletion techniques enable the partial or complete removal of the gene thereby eliminating its expression. In such methods, deletion of the gene is accomplished by homologous recombination using a plasmid that has been constructed to contiguously contain the 5′ and 3′ regions flanking the gene.

The mutant filamentous fungal cell can also be constructed by any RNA-guided DNA endonuclease using, for example, MAD7 (U.S. Pat. No. 9,982,279), MAD2 (U.S. Pat. No. 9,982,279), Cas9 (Doudna et al., 2014, Science 346: 1258096), “dead” Cas9 (dcas9; Qi et al., 2013, Cell 152(5): 1173), Cas9 nickase (Satomura et al. 2017, Sci. Rep. 7(1):2095), or Cpf1 endonuclease (Zetsche et al. 2015, Cell 163(3): 759), directed to the nucleotide sequence of the gene by a suitably designed guide RNA.

The mutant filamentous fungal cell can also be constructed by introducing, substituting, and/or deleting one or more nucleotides in the gene or a control sequence thereof required for the transcription or translation thereof. For example, nucleotides can be inserted or removed for the introduction of a stop codon, the removal of the start codon, or a frame-shift of the open reading frame. Such a modification can be accomplished by site-directed mutagenesis or PCR generated mutagenesis in accordance with methods known in the art. See, for example, Botstein and Shortie, 1985, Science 229: 4719; Lo et al., 1985, Proceedings of the National Academy of Sciences USA 81: 2285; Higuchi et al., 1988, Nucleic Acids Research 16: 7351; Shimada, 1996, Meth. Mol. Biol. 57: 157; Ho et al., 1989, Gene 77: 61; Horton et al., 1989, Gene 77: 61; and Sarkar and Sommer, 1990, BioTechniques 8: 404.

The mutant filamentous fungal cell can also be constructed by gene disruption techniques by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct can be simply a selectable marker gene accompanied by 5′ and 3′ regions homologous to the gene. The selectable marker enables identification of transformants containing the disrupted gene.

The mutant filamentous fungal cell can also be constructed by the process of gene conversion (see, for example, Iglesias and Trautner, 1983, Molecular General Genetics 189: 73-76). For example, in the gene conversion method, a nucleotide sequence corresponding to the gene is mutagenized in vitro to produce a defective nucleotide sequence, which is then transformed into the filamentous fungal cell to produce a defective gene. By homologous recombination, the defective nucleotide sequence replaces the endogenous gene. It may be desirable that the defective nucleotide sequence also comprises a marker for selection of transformants containing the defective gene.

The mutant filamentous fungal cell can also be constructed by established anti-sense techniques using a nucleotide sequence complementary to the nucleotide sequence of the gene (Parish and Stoker, 1997, FEMS Microbiology Letters 154: 151-157). More specifically, expression of the gene can be reduced or inactivated by introducing a nucleotide sequence complementary to the nucleotide sequence of the gene, which can be transcribed in the cell and is capable of hybridizing to the mRNA produced in the cell. Under conditions allowing the complementary anti-sense nucleotide sequence to hybridize to the mRNA, the amount of protein translated is thus reduced or eliminated.

The mutant filamentous fungal cell can also be constructed by established RNA interference (RNAi) techniques (see, for example, WO 2005/056772 and WO 2008/080017).

The mutant filamentous fungal cell can be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the gene can be performed by subjecting the parent cell to mutagenesis and screening for mutant cells in which expression of the gene has been reduced or inactivated. The mutagenesis, which can be specific or random, can be performed, for example, by use of a suitable physical or chemical mutagenizing agent, use of a suitable oligonucleotide, or subjecting the DNA sequence to PCR generated mutagenesis. Furthermore, the mutagenesis can be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent cell to be mutagenized in the presence of the mutagenizing agent of choice under suitable conditions, and selecting for mutants exhibiting reduced or no expression of the gene.

Modification of expression of a gene encoding a transcription factor of the present invention can be measured by one of ordinary skill in the art through analysis of selected mRNA or transcript levels by well-known means, for example, quantitative real-time PCR (qRT-PCR), Northern blot hybridization, global gene expression profiling using cDNA or oligo array hybridization, or deep RNA sequencing (RNA-seq). Alternatively, modification of a gene encoding a transcription factor of the present invention can be determined by fungal spore PCR using a locus-specific primer as described in Example 9.

In one aspect, the mutant is partially deficient in the production of the transcription factor compared to the parent filamentous fungal cell without the modification when cultivated under identical conditions. In a preferred aspect, the mutant produces at least 25% less, more preferably at least 50% less, even more preferably at least 75% less, and most preferably at least 95% less transcription factor than the parent filamentous fungal cell without the modification when cultivated under identical conditions.

In another aspect, the mutant is completely deficient in the production of the transcription factor compared to the parent filamentous fungal cell without the modification when cultivated under identical conditions. In other words, the gene encoding the transcription factor is inactivated (e.g., deletion, disruption, etc. of the gene).

### Polypeptides of Interest

The polypeptide of interest can be any polypeptide native or foreign (heterologous) to the mutant filamentous fungal cell whose coding sequence is under the transcriptional control of a promoter regulated by a transcription factor of the present invention. The promoter can be native or heterologous to the coding sequence of the polypeptide of interest. The polypeptide can be encoded by a single gene or two or more genes. The term “heterologous polypeptide” is defined herein as a polypeptide that is not native to the cell; a native polypeptide in which structural modifications have been made to alter the native polypeptide, e.g., the protein sequence of a native polypeptide; or a native polypeptide whose expression is quantitatively altered as a result of a manipulation of the polynucleotide or host cell by recombinant DNA techniques, e.g., a different promoter, multiple copies of a DNA encoding the polypeptide. Thus, the present invention also encompasses, within the scope of the term “heterologous polypeptides,” such recombinant production of native polypeptides, to the extent that such expression involves the use of genetic elements not native to the filamentous fungal cell, or use of native elements that have been manipulated to function in a manner that do not normally occur in the filamentous fungal cell.

In one aspect, the polypeptide is native to the filamentous fungal cell. In another aspect, the polypeptide is heterologous to the filamentous fungal cell.

The polypeptide can be any polypeptide having a biological activity of interest. The term “polypeptide” is not meant herein to refer to a specific length of the encoded product and, therefore, encompasses peptides, oligopeptides, and proteins. The term “polypeptide” also encompasses two or more polypeptides combined to form the encoded product. Polypeptides also include fusion polypeptides, which comprise a combination of partial or complete polypeptide sequences obtained from at least two different polypeptides wherein one or more can be heterologous to the filamentous fungal cell. Polypeptides further include hybrid polypeptides comprising domains from two or more polypeptides, e.g., a binding domain from one polypeptide and a catalytic domain from another polypeptide. The domains may be fused at the N-terminus or the C-terminus.

In one aspect, the polypeptide is an antibody, an antigen, an antimicrobial peptide, an enzyme, a growth factor, a hormone, an immunomodulator, a neurotransmitter, a receptor, a reporter protein, a structural protein, or a transcription factor.

In another aspect, the polypeptide is an oxidoreductase, a transferase, a hydrolase, a lyase, an isomerase, or a ligase. In another aspect, the polypeptide is an acetylmannan esterase, acetylxylan esterase, aminopeptidase, alpha-amylase, arabinanase, arabinofuranosidase, carbohydrase, carboxypeptidase, catalase, cellobiohydrolase, cellulase, chitinase, coumaric acid esterase, cyclodextrin glycosyltransferase, cutinase, cyclodextrin glycosyltransferase, deamidase, deoxyribonuclease, dispersin, endoglucanase, esterase, feruloyl esterase, AA9 lytic polysaccharide monooxygenase, alpha-galactosidase, beta-galactosidase, glucocerebrosidase, glucose oxidase, alpha-glucosidase, beta-glucosidase, glucuronidase, glucuronoyl esterase, haloperoxidase, hemicellulase, invertase, isomerase, laccase, ligase, lipase, lysozyme, mannanase, mannosidase, mutanase, oxidase, pectinolytic enzyme, peroxidase, phosphodiesterase, phospholipase, phytase, phenoloxidase, polyphenoloxidase, proteolytic enzyme, ribonuclease, alpha-1,6-transglucosidase, transglutaminase, urokinase, xanthanase, xylanase, or beta-xylosidase.

In another aspect, the polypeptide is a cellulase. In another aspect, the cellulase is selected from the group consisting of an endoglucanase, a cellobiohydrolase, and a beta-glucosidase.

In another aspect, the polypeptide is a hemicellulase. In another aspect, the hemicellulase is selected from the group consisting of a xylanase, an acetylxylan esterase, a feruloyl esterase, an arabinofuranosidase, a xylosidase, and a glucuronidase.

In another aspect, the polypeptide is an endoglucanase. In another aspect, the polypeptide is a cellobiohydrolase. In another aspect, the polypeptide is a beta-glucosidase. In another aspect, the polypeptide is an AA9 lytic polysaccharide monooxygenase. In another aspect, the polypeptide is a xylanase. In another aspect, the polypeptide is a beta-xylosidase. In another aspect, the polypeptide is an acetyxylan esterase. In another aspect, the polypeptide is a feruloyl esterase. In another aspect, the polypeptide is an arabinofuranosidase. In another aspect, the polypeptide is a glucuronidase. In another aspect, the polypeptide is an acetylmannan esterase. In another aspect, the polypeptide is an arabinanase. In another aspect, the polypeptide is a coumaric acid esterase. In another aspect, the polypeptide is a galactosidase. In another aspect, the polypeptide is a glucuronoyl esterase. In another aspect, the polypeptide is a mannanase. In another aspect, the polypeptide is a mannosidase.

In the methods of the present invention, the mutant filamentous fungal cell is a recombinant cell, comprising a polynucleotide encoding a heterologous polypeptide, which is advantageously used in the recombinant production of the polypeptide. The cell is preferably transformed with a nucleic acid construct or an expression vector comprising the polynucleotide encoding the heterologous polypeptide followed by integration of the vector into the chromosome. “Transformation” means introducing a vector comprising the polynucleotide into a host cell so that the vector is maintained as a chromosomal integrant or as a self-replicating extra-chromosomal vector. Integration is generally considered to be an advantage as the polynucleotide is more likely to be stably maintained in the cell. Integration of the vector into the chromosome can occur by homologous recombination, non-homologous recombination, or transposition.

The polynucleotide encoding a heterologous polypeptide can be obtained from any prokaryotic, eukaryotic, or other source, e.g., archaeabacteria. For purposes of the present invention, the term “obtained from” as used herein in connection with a given source shall mean that the polypeptide is produced by the source or by a cell in which a gene from the source has been inserted.

The techniques used to isolate or clone a polynucleotide encoding a polypeptide of interest are known in the art and include isolation from genomic DNA, preparation from cDNA, or a combination thereof. The cloning of such a polynucleotide from such genomic DNA can be effected, e.g., by using the well-known polymerase chain reaction (PCR). See, for example, Innis et al., 1990, PCR Protocols: A Guide to Methods and Application, Academic Press, New York. The cloning procedures may involve excision and isolation of a desired nucleic acid fragment comprising the polynucleotide encoding the polypeptide, insertion of the fragment into a vector molecule, and incorporation of the recombinant vector into a mutant filamentous fungal cell of the present invention where multiple copies or clones of the polynucleotide will be replicated. The polynucleotide can be of genomic, cDNA, RNA, semisynthetic, synthetic origin, or any combinations thereof.

An isolated polynucleotide encoding a heterologous polypeptide can be manipulated in a variety of ways to provide for expression of the polypeptide in a mutant filamentous fungal cell of the present invention. Manipulation of the polynucleotide's sequence prior to its insertion into a vector can be desirable or necessary depending on the expression vector. The techniques for modifying polynucleotide sequences utilizing recombinant DNA methods are well known in the art.

A nucleic acid construct comprising a polynucleotide encoding a polypeptide can be operably linked to one or more control sequences capable of directing expression of the coding sequence in a mutant filamentous fungal cell of the present invention under conditions compatible with the control sequences.

The control sequence can be an appropriate promoter sequence regulated by a transcription factor of the present invention. The promoter sequence contains transcriptional control sequences that mediate expression of the polypeptide. The promoter can be any nucleotide sequence that shows transcriptional activity in the filamentous fungal cell, including mutant, truncated, and hybrid promoters, and can be obtained from genes encoding extracellular or intracellular polypeptides either native or heterologous (foreign) to the filamentous fungal cell.

In one aspect, the promoter is a promoter from a cellulase gene regulated by a transcription factor of the present invention. In another aspect, the promoter is a promoter from a hemicellulase gene regulated by a transcription factor of the present invention. The cellulase gene can be an endoglucanase, a cellobiohydrolase, or a beta-glucosidase gene. The hemicellulase gene can be an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, or a xylosidases gene.

In one aspect, the cellulase gene or hemicellulase gene can be obtained from an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria cell.

In another aspect, the cellulase gene or hemicellulase gene can be obtained from an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Talaromyces emersonii, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride cell.

In a preferred aspect, the cellobiohydrolase gene is a cellobiohydrolase I gene. In a more preferred aspect, the cellobiohydrolase I gene is a Trichoderma cellobiohydrolase I gene. In a most preferred aspect, the cellobiohydrolase I gene is a Trichoderma reesei cellobiohydrolase I gene.

In another preferred aspect, the cellobiohydrolase gene is a cellobiohydrolase II gene. In a more preferred aspect, the cellobiohydrolase II gene is a Trichoderma cellobiohydrolase II gene. In a most preferred aspect, the cellobiohydrolase II gene is a Trichoderma reesei cellobiohydrolase II gene.

Examples of suitable promoters for directing the transcription of the nucleic acid constructs in the methods of the present invention are promoters obtained from the genes for Trichoderma reesei beta-glucosidase, Trichoderma reesei AA9 lytic polysaccharide monoxygenase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei swollenin 1-, Trichoderma reesei glycosyl hydrolase family 11 xylanase I, Trichoderma reesei glycosyl hydrolase family 11 xylanase II, Trichoderma reesei family 10 xylanase I, Trichoderma reesei family 10 xylanase II, Trichoderma reesei glycosyl hydrolase family 10 xylanase III, Trichoderma reesei glycosyl hydrolase family 30 xylanase IV, and Trichoderma reesei beta-xylosidase; and mutant, truncated, and hybrid promoters thereof.

The control sequence may also be a suitable transcription terminator sequence, a sequence recognized by a mutant filamentous fungal cell of the present invention to terminate transcription. The terminator sequence is operably linked to the 3′ terminus of the nucleotide sequence encoding the heterologous polypeptide. Any terminator that is functional in a filamentous fungal cell can be used in the present invention.

Preferred terminators are obtained from the genes for Aspergillus oryzae TAKA amylase, Aspergillus niger glucoamylase, Aspergillus nidulans anthranilate synthase, Aspergillus niger alpha-glucosidase, Fusarium oxysporum trypsin-like protease, Trichoderma reesei beta-glucosidase, Trichoderma reesei AA9 lytic polysaccharide monoxygenase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei swollenin 1-, Trichoderma reesei glycosyl hydrolase family 11 xylanase I, Trichoderma reesei glycosyl hydrolase family 11 xylanase II, Trichoderma reesei family 10 xylanase I, Trichoderma reesei family 10 xylanase II, Trichoderma reesei glycosyl hydrolase family 10 xylanase Ill, Trichoderma reesei glycosyl hydrolase family 30 xylanase IV, and Trichoderma reesei beta-xylosidase

The control sequence may also be a suitable leader sequence, a nontranslated region of a mRNA that is important for translation by a mutant filamentous fungal cell of the present invention. The leader sequence is operably linked to the 5′ terminus of the nucleotide sequence encoding the heterologous polypeptide. Any leader sequence that is functional in the filamentous fungal cell can be used in the present invention.

Preferred leaders for filamentous fungal cells are obtained from the genes for Aspergillus oryzae TAKA amylase and Aspergillus nidulans triose phosphate isomerase.

The control sequence may also be a polyadenylation sequence, a sequence operably linked to the 3′ terminus of the nucleotide sequence and, when transcribed, is recognized by the filamentous fungal cell as a signal to add polyadenosine residues to transcribed mRNA. Any polyadenylation sequence that is functional in the filamentous fungal cell can be used in the present invention.

Preferred polyadenylation sequences for filamentous fungal cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence can be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of the filamentous fungal cell, i.e., secreted into a culture medium, can be used in the present invention.

Effective signal peptide coding regions for the filamentous fungal cell are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to a mature, active polypeptide by catalytic or autocatalytic cleavage of the propeptide from the propolypeptide. The propeptide coding region can be obtained from genes for Saccharomyces cerevisiae alpha-factor, Rhizomucor miehei aspartic proteinase, and Myceliophthora thermophila laccase (WO 95/33836).

Where both signal peptide and propeptide sequences are present at the amino terminus of a polypeptide, the propeptide sequence is positioned next to the amino terminus of a polypeptide and the signal peptide sequence is positioned next to the amino terminus of the propeptide sequence.

The nucleic acid constructs may also comprise one or more polynucleotides that encode one or more factors that are advantageous for directing expression of the heterologous polypeptide, e.g., a transcriptional activator (e.g., a trans-acting factor), a chaperone, and a processing protease. Any factor that is functional in the filamentous fungal cell can be used in the present invention. The nucleic acids encoding one or more of these factors are not necessarily in tandem with the nucleotide sequence encoding the heterologous polypeptide.

In the methods of the present invention, a recombinant expression vector comprising a nucleotide sequence, a promoter, and transcriptional and translational stop signals can be used for the recombinant production of a polypeptide of interest. The various nucleic acids and control sequences described herein can be joined together to produce a recombinant expression vector that may include one or more convenient restriction sites to allow for insertion or substitution of the nucleotide sequence encoding the polypeptide at such sites. Alternatively, the nucleotide sequence can be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector can be any vector (e.g., a plasmid or virus) that can be conveniently subjected to recombinant DNA procedures and can bring about expression of the nucleotide sequence. The choice of the vector will typically depend on its compatibility with the filamentous fungal cell into which the vector is to be introduced. The vector can be a linear or closed circular plasmid.

The vector can be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector can be one that, when introduced into the filamentous fungal cell, is integrated into the genome and replicated together with the chromosome(s) into which it has been integrated. Furthermore, a single vector or plasmid or two or more vectors or plasmids that together contain the total DNA to be introduced into the genome of the filamentous fungal cell, or a transposon, can be used.

The vector preferably contains one or more selectable markers that permit easy selection of a transformed filamentous fungal cell. A selectable marker is a gene the product of which provides for biocide or viral resistance, resistance to heavy metals, prototrophy to auxotrophs, and the like.

Examples of selectable markers for use in the filamentous fungal cell include, but are not limited to, amdS (acetamidase), argB (ornithine carbamoyltransferase), bar (phosphinothricin acetyltransferase), hpt (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in the filamentous fungal cell are the amdS gene of Aspergillus nidulans and the bar gene of Streptomyces hygroscopicus.

The vectors preferably contain an element(s) that permits integration of the vector into the host cell's genome or autonomous replication of the vector in the cell independent of the genome.

For integration into the genome of the filamentous fungal cell, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the filamentous fungal cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity to the corresponding target sequence to enhance the probability of homologous recombination. The integrational elements can be any sequence that is homologous with the target sequence in the genome of the filamentous fungal cell. Furthermore, the integrational elements can be non-encoding or encoding nucleotide sequences. On the other hand, the vector can be integrated into the genome of the filamentous fungal cell by non-homologous recombination.

For autonomous replication, the vector may further comprise an origin of replication enabling the vector to replicate autonomously in the filamentous fungal cell. The origin of replication can be any plasmid replicator mediating autonomous replication that functions in a cell. The term “origin of replication” or “plasmid replicator” is defined herein as a nucleotide sequence that enables a plasmid or vector to replicate in vivo.

Examples of origins of replication useful in the filamentous fungal cell are AMA1 and ANSI (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

The procedures used to ligate the elements described herein to construct the recombinant expression vectors are well known to one skilled in the art (see, e.g., J. Sambrook, E. F. Fritsch, and T. Maniatis, 1989, Molecular Cloning, A Laboratory Manual, 2d edition, Cold Spring Harbor, N.Y.).

A vector can be introduced, e.g., by transformation, into the filamentous fungal cell so that the vector is maintained as a chromosomal integrant or as a self-replicating extra-chromosomal vector. Integration is generally considered to be an advantage as the nucleotide sequence is more likely to be stably maintained in the cell. Integration of the vector into the chromosome occurs by homologous recombination, non-homologous recombination, or transposition.

The introduction of an expression vector into the filamentous fungal cell may involve a process consisting of protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

### Methods of Producing a Polypeptide of Interest

The present invention also relates to a method of producing a polypeptide of interest, comprising (a) cultivating a mutant filamentous fungal cell of the present invention for production of the polypeptide of interest, and optionally (b) recovering the polypeptide of interest.

The mutant filamentous fungal cell is cultivated in a nutrient medium suitable for production of the polypeptide using methods known in the art. For example, the filamentous fungal cell can be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide of interest can be detected using methods known in the art that are specific for the polypeptide. These detection methods include, but are not limited to, use of specific antibodies, formation of an enzyme product, or disappearance of an enzyme substrate. For example, an enzyme assay can be used to determine the activity of the polypeptide.

The increase in productivity of the polypeptide of interest by the mutant filamentous fungal cell can be measured using the methods above. The increase in expression of the gene encoding the polypeptide of interest can be determined by analysis of selected mRNA or transcript levels by well-known means, for example, quantitative real-time PCR (qRT-PCR), Northern blot hybridization, global gene expression profiling using cDNA or oligo array hybridization, or deep RNA sequencing (RNA-seq).

The polypeptide can be recovered using methods known in the art. For example, the polypeptide can be recovered from the nutrient medium by conventional procedures including, but not limited to, collection, centrifugation, filtration, extraction, spray-drying, evaporation, or precipitation. In one aspect, a whole fermentation broth comprising the polypeptide is recovered.

The polypeptide can be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Methods of Producing a Transcription Factor

The present invention also relates to isolated polynucleotides encoding a transcription factor of the present invention, as described herein.

The techniques used to isolate or clone a polynucleotide are known in the art and include isolation from genomic DNA or cDNA, or a combination thereof, as described supra for a polypeptide of interest. The polynucleotide can be cloned from a strain of Trichoderma, or a related organism and thus, for example, can be an allelic or species variant of the transcription factor encoding region of the polynucleotide.

The present invention also relates to nucleic acid constructs and expression vectors comprising a polynucleotide encoding a transcription factor of the present invention operably linked to one or more control sequences that direct the expression of the coding sequence in a suitable host cell under conditions compatible with the control sequences. The polynucleotide encoding a transcription factor can be manipulated in a manner as described supra for a polynucleotide encoding a polypeptide of interest.

The present invention also relates to recombinant host cells, comprising a polynucleotide of the present invention operably linked to one or more control sequences that direct the production of a transcription factor of the present invention. A nucleic acid construct or expression vector comprising the polynucleotide is introduced into a host cell so that the construct or vector is maintained as a chromosomal integrant or as a self-replicating extra-chromosomal vector as described earlier.

The recombinant host cell can be any filamentous fungal cell. The filamentous fungal cell can be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocaffimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host can be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell. Filamentous fungal cells can be transformed using the procedures described supra.

The present invention also relates to methods of producing a transcription factor of the present invention, comprising (a) cultivating a recombinant host cell of the present invention under conditions conducive for production of the transcription factor; and optionally, (b) recovering the transcription factor. The host cells are cultivated in a nutrient medium suitable for production of the transcription factor, the transcription factor is detected and recovered using methods known in the art such as those described supra for a polypeptide of interest

The present invention is further described by the following examples that should not be construed as limiting the scope of the invention.

### EXAMPLES

### Strains

Trichoderma reesei BTR213 is described in WO 2013/086633.

Trichoderma reesei strain AgJg216-2B51 is a ku70 disrupted and paracelsin synthetase (pars) deleted strain of T. reesei BTR213 comprising four copies of an Acremonium alcalophilum GH25 lysozyme gene (SEQ ID NO: 57 for the DNA sequence and SEQ ID NO: 58 for the deduced amino acid sequence) under the transcriptional control of the Trichoderma reesei cellobiohydrolase I (cbh1) promoter and terminator.

Trichoderma reesei strain GMer62-1A9 is a is a ku70 disrupted and paracelsin synthetase (pars) gene deleted strain of T. reesei BTR213.

### Media and Solutions

0.2 M Citric acid was composed of 38.424 g of citric acid and deionized water to 1 liter. The solution was filter sterilized.

CMC plates were composed of 0.5% sodium carboxymethylcellulose (AQUALON™ Ashland, Inc), 10 ml of 50× Trace Elements Solution, 125 ml of 2× Mineral Salt Solution, 250 ml of 0.1 M phosphate-citrate buffer, 0.5 ml of 5 M Urea, 10 g of Noble agar, and deionized water to 500 ml.

Congo Red Solution was composed of 2.5 g of Congo red and deionized water to 1 liter.

COVE plates were composed of 218 g of sorbitol, 20 g of agar, 20 ml of COVE salts solution, 10 mM acetamide, 15 mM CsCl, and deionized water to 1 liter. The solution was adjusted to pH 7.0 before autoclaving.

COVE salts solution was composed of 26 g of KCl, 26 g of MgSO₄.7H₂O, 76 g of KH₂PO₄, 50 ml COVE trace metals solution, and deionized water to 1 liter.

COVE trace metals solution was composed of 0.04 g of Na₂B₄O₇.10H₂O, 0.4 g of CuSO₄.5H₂O, 1.2 g of FeSO₄.7H₂O, 0.7 g of MnSO₄.H₂O, 0.8 g of Na₂MoO₂.2H₂O, 10 g of ZnSO₄.7H₂O, and deionized water to 1 liter.

COVE2 μlates were composed of 30 g of sucrose, 20 ml of COVE salts solution, 10 ml 1 M acetamide, 25 g of Noble agar, and deionized water to 1 liter.

Fermentation Batch Medium was composed of 24 g of dextrose, 40 g of soy meal, 8 g of (NH₄)₂SO₄, 3 g of K₂HPO₄, 8 g of K₂SO₄, 3 g of CaCO₃, 8 g of MgSO₄.7H₂O, 1 g of citric acid, 8.8 ml of 85% phosphoric acid, 1 ml of anti-foam, 14.7 ml of trace metals solution, and deionized water to 1 liter. The trace metals solution was composed of 26.1 g of FeSO₄.7H₂O, 5.5 g of ZnSO₄.7H₂O, 6.6 g of MnSO₄.H₂O, 2.6 g of CuSO₄.5H₂O, 2 g of citric acid, and deionized water to 1 liter.

50× Lactose/peptone solution was composed of 20 g of lactose, 2 g of peptone, and deionized water to 100 ml.

LB+Amp medium was composed of 10 g of tryptone, 5 g of yeast extract, 5 g of sodium chloride, 50 mg of ampicillin (filter sterilized, added after autoclaving), and deionized water to 1 liter.

MA medium was composed of 10 g of lactose, 1 g of Bacto peptone, 2.8 g of (NH₄)₂SO₄, 4 g of KH₂PO₄, 0.6 g of MgSO₄.7H₂O, 0.8 g of CaCl₂.2H₂O, 20 ml of MA trace elements solution, 500 ml of phosphate-citrate buffer, and deionized water to 1 liter. The MA trace elements solution was composed of 250 mg of FeSO₄.7H₂O, 85 mg of MnSO₄.H₂O, 70 mg of ZnSO₄.7H₂O, 100 mg of CaCl₂.2H₂O, and deionized water to 1 liter; pH adjusted to 2.0.

Mandels-Andreotti Medium was composed of 20 ml of 50× Trace Elements Solution, 250 ml of 2× Mineral Salt Solution, 500 ml of 0.1 M phosphate-citrate buffer, 1 ml of 5 M urea, 20 ml of 50× lactose/peptone solution, and deionized water to 1 liter.

MEX plates were composed of 15 g of malt extract, 0.5 g of peptone, 7.5 g of Noble agar, and deionized water to 500 ml. The solution is autoclaved for 20 minutes, allowed to cool down to about 55° C. before pouring it onto 150 mm petri dishes; 50 ml per plate.

2× Mineral Salt Solution was composed of 5.6 g of (NH₄)₂SO₄ (43.8 mM), 8.0 g KH₂PO₄ (58.32 mM), 1.2 g of MgSO₄.7H₂O (4.86 mM), 1.6 g of CaCl₂.2H₂O (10.88 mM) and deionized water to 1 liter. The solution was sterilized by autoclaving.

PDA plates were composed of 39 g of potato dextrose agar (Difco) and deionized water to 1 liter. The solution was sterilized by autoclaving.

PDA overlay medium was composed of 39 g of potato dextrose agar (Difco) and deionized water to 1 liter. The solution was sterilized by autoclaving. The autoclaved medium was melted in a microwave and then tempered to 55° C. before use.

PEG buffer was composed of 50% polyethylene glycol (PEG) 4000, 10 mM Tris-HCl pH 7.5, and 10 mM CaCl₂ in deionized water.

0.1 M Phosphate-citrate buffer was composed of 14.19 g of Na₂HPO₄ anhydrous (0.1 M) dissolved in 500 ml of deionized water. The pH of the solution was adjusted to pH 5.0 with 0.2 M citric acid and filter sterilized. The final volume of the solution was not adjusted to one liter.

Shake Flask Medium was composed of 20 g of glycerol, 10 g of soy meal, 1.5 g of (NH₄)₂SO₄, 2 g of KH₂PO₄, 0.2 g of CaCl₂, 0.4 g of MgSO₄.7H₂O, 0.2 ml of trace metals solution, and deionized water to 1 liter. The trace metals solution was composed of 26.1 g of FeSO₄.7H₂O, 5.5 g of ZnSO₄.7H₂O, 6.6 g of MnSO₄.H₂O, 2.6 g of CuSO₄.5H₂O, 2 g of citric acid, and deionized water to 1 liter.

SOC medium was composed of 20 g of tryptone, 5 g of yeast extract, 0.5 g of NaCl, 10 ml of 250 mM KCl, and deionized water to 1 liter.

STC was composed of 1 M sorbitol, 10 mM Tris pH 7.5, and 10 mM CaCl₂ in deionized water.

TAE buffer was composed of 4.84 g of Tris base, 1.14 ml of glacial acetic acid, 2 ml of 0.5 M EDTA pH 8.0, and deionized water to 1 liter.

50× Trace Elements Solution was composed of 250 mg of FeSO₄.7H₂O (0.9 mM), 85 mg of MnSO₄.H₂O (0.31 mM), 70 mg of ZnSO₄.7H₂O (0.24 mM), 100 mg of CaCl₂.2H₂O (0.68 mM), and deionized water to 1 liter. The pH of the solution was adjusted to pH 2 with HCl. The solution was sterilized by autoclaving.

Trichoderma Minimal Medium (TrMM) plates were composed of 20 ml of COVE salts solution, 0.6 g of CaCl₂.2H₂O, 6 g of (NH₄)₂SO₄, 25 g of Noble agar, and deionized water to 480 ml. After the solution was autoclaved and cooled to 55° C., 20 ml of filter sterilized 50% glucose was added.

5 M Urea was composed of 15.015 g of urea in 50 ml of deionized water and filter sterilized.

2XYT+Amp plates were composed of 16 g of tryptone, 10 g of yeast extract, 5 g of NaCl, 15 g of Bacto agar, 1 ml of ampicillin at 100 mg/ml, and deionized water to 1 liter.

YP medium was composed of 1% yeast extract and 2% peptone in deionized water.

### Example 1: Genomic DNA Extraction from Trichoderma reesei

Trichoderma reesei was grown in 50 ml of YP medium supplemented with 2% glucose (w/v) in a 250 ml baffled shake flask at 28° C. for 2 days with agitation at 200 rpm. Mycelia from the cultivation were collected using a MIRACLOTH® (EMD Chemicals Inc.) lined funnel, squeeze-dried, and then transferred to a pre-chilled mortar and pestle. Each mycelia preparation was ground into a fine powder and kept frozen with liquid nitrogen. A total of 1-2 g of powder was transferred to a 50 ml tube and genomic DNA was extracted from the ground mycelial powder using a DNEASY® Plant Maxi Kit (QIAGEN Inc.). Five ml of AP1 Buffer (QIAGEN Inc.) pre-heated to 65° C. were added to the 50 ml tube followed by 10 μl of RNase A 100 mg/ml stock solution (QIAGEN Inc.), and incubated for 2-3 hours at 65° C. A total of 1.8 ml of AP2 Buffer (QIAGEN Inc.) was added and centrifuged at 3000-5000×g for 5 minutes. The supernatant was decanted into a QIAshredder Maxi Spin Column (QIAGEN Inc.) placed in a 50 ml collection tube, and centrifuged at 3000-5000×g for 5 minutes at room temperature (15-25° C.) in a swing-out rotor. The flow-through in the collection tube was transferred, without disturbing the pellet, into a new 50 ml tube. A 1.5 ml volume of AP3/E Buffer (QIAGEN Inc.) was added to the cleared lysate, and mixed immediately by vortexing. The sample (maximum 15 ml), including any precipitate that may have formed, was pipetted into a DNEASY® Maxi Spin Column (QIAGEN Inc.) placed in a 50 ml collection tube and centrifuged at 3000-5000×g for 5 minutes at room temperature (15-20° C.) in a swing-out rotor. The flow-through was discarded. Twelve ml of AW Buffer (QIAGEN Inc.) were added to the DNEASY® Maxi Spin Column, and centrifuged for 10 minutes at 3000-5000×g to dry the membrane. The flow-through and collection tube were discarded. The DNEASY® Maxi Spin Column was transferred to a new 50 ml tube. One-half ml of AE Buffer (QIAGEN Inc.), pre-heated to 65° C., was pipetted directly onto the DNEASY® Maxi Spin Column membrane, incubated for 5 minutes at room temperature (15-25° C.), and then centrifuged for 5 minutes at 3000-5000×g to elute the genomic DNA. The concentration and purity of the genomic DNA was determined by measuring the absorbance at 260 nm and 280 nm.

### Example 2: Trichoderma reesei Protoplast Generation and Transformation

Protoplast preparation and transformation of Trichoderma reesei were performed using a protocol similar to Penttila et al., 1987, Gene 61: 155-164. Briefly, T. reesei was cultivated in 25 ml of YP medium supplemented with 2% (w/v) glucose and 10 mM uridine at 27° C. for 17 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 100 ml of 1.2 M sorbitol containing 5 mg of YATALASE™ Enzyme (Takara Bio USA, Inc.) per ml and 0.36 units of chitinase (Sigma Chemical Co.) per ml for 60-75 minutes at 34° C. with gentle shaking at 90 rpm. Protoplasts were collected by centrifugation at 834×g for 7 minutes and washed twice with cold 1.2 M sorbitol. The protoplasts were counted using a hemocytometer and re-suspended to a final concentration of 1×10⁸ protoplasts per ml of STC.

Approximately 1-10 μg of DNA were added to 100 μl of the protoplast solution and mixed gently. PEG buffer (250 μl) was added, and the reaction was mixed and incubated at 34° C. for 30 minutes. STC (3 ml) was then added, and the reaction was mixed and then spread onto PDA plates supplemented with 1 M sucrose for hygromycin selection. After incubation at 30° C. for 16 hours, 20 ml of PDA overlay medium supplemented with 35 μg/ml of hygromycin B (Thermo Fisher Scientific) were added to each plate. The plates were incubated at 30° C. for 4-7 days.

### Example 3: Generation of Trichoderma reesei BTR213 Cellulase Non-Producing (Cel−) and Cellulase Producing (Cel+) Isolates

Cellulase non-producing (cel−) isolates and cellulase producing (cel+) isolates of Trichoderma reesei BTR213 were generated by fermenting T. reesei BTR213 in a 14-liter pilot scale fed-batch fermentor as described in WO 2013/086633. At 168 hours from the start of the fermentation, cells were collected and tested for their ability to produce cellulase by plating on acid swollen cellulose plates as described in WO 2013/086633. Cells forming zones of clearing on the acid swollen cellulose plates were identified as cellulase producing (cel+) isolates and cells forming no clearing zones were identified as cellulase non-producing (cel−) isolates.

### Example 4: Cultivation of Trichoderma reesei BTR213 Cellulase Non-Producing (Cel−) and Cellulase Producing (Cel+) Isolates for RNA Sequencing Analysis

The Trichoderma reesei BTR213 cellulase non-producing (cel−) and cellulase producing (cel+) isolates (Example 3) were grown in 200 ml of MA medium in 1 liter shake flasks for 24, 48, 72, 96, and 120 hours at 30° C. with shaking at 180 rpm. Each cultivation was performed in triplicate. Mycelia at each time point and each replicate were separately harvested by filtration using MIRACLOTH®, washed twice in deionized water, and frozen under liquid nitrogen. Frozen mycelia were ground by mortar and pestle to a fine powder. Total RNA was isolated using a TRIZOL® Plus RNA Purification Kit (Life Technologies/Invitrogen) according to the manufacturers protocol.

### Example 5: RNAseq Analysis of Trichoderma reesei BTR213 Cellulase Non-Producing (Cel−) and Cellulase Producing (Cel+) Isolates

TruSeq RNAseq libraries were constructed from RNA isolated from three biological replicates each for two conditions—cel+ (48 hours) and cel− (48 hours) as described in Example 4. The libraries were sequenced using paired 150 base pair Illumina reads on the NEXTSEQ™ (Illumina Inc.) platform. Approximately 10-12 million reads were generated per sample. In-silico analyses were performed in CLC Genomics Server version 7 (CLCBio Genomics, QIAGEN). Sequences were trimmed based on quality and adapter sequence removed. Trimmed sequences were mapped to the Trichoderma reesei Rut-C30 gene sequences (Trichoderma reesei Rut-C30 genome database; Jourdier et al., 2017, Biotechnol. Biofuels 10: 151) using the RNAseq module implemented in CLC Genomics Server version 7 (mismatch=2, similarity=0.8, max matches_per_read=10). Total read counts summarized per gene were used for downstream analysis. Replicate quality was assessed using boxplots and principal components analysis. Differential expression analyses (DGE) was performed as a contrast analysis between the 48 hours cel+ versus cel− samples using the EdgeR-based methods implemented in CLC Bio Genomics version 7 (Robinson et al., 2010, Bioinformatics 26 (1): 139-140). Significance was assessed by filtering using a FDR-corrected p-value threshold of 0.05. This subset of significant DGE genes were binned into groups using k-means clustering (bin=20). GO terms and pathway mappings using EC number annotation were used to add functional annotation. Source files for these annotations were downloaded from the Joint Genome Institute's Trichoderma reesei QM6A genome database, (JGI Trichoderma reesei genome database v. 2.0; Martinez et al., 2008, Nature Biotechnol. 26: 553-560). Custom-curated annotations received from the Mach Lab were used to annotate known transcription factors. Custom PERL scripts and database queries were used to combine the various annotation sources to make a final list of annotated transcription factor genes.

A listing of the transcription factor genes is shown in Table 1 (SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55, for the DNA sequence and SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, and 124, for the amino acid sequence). JGI Protein Nos. correspond to the annotated genes from the Joint Genome Institute's Trichoderma reesei Rut-C30 genome database, (JGI Trichoderma reesei genome database v.1.0; Jourdier et al., 2017, Biotechnol. Biofuels 10: 151).

Models for transcription factors 70883 and 132448 were split in two to reflect conflicting data obtained about these models from the RNAseq data.

TABLE 1

| JGI | | SEQ |
|---|---|---|
| Protein | | ID |
| No. | JGI T. reesei Rut-C30 Annotation | NO |
| 92949 | Signal transduction response regulator, pH-responsive, | 2 |
| | Pall/Rim9 | |
| 70883 | Zn(2)-C6 fungal-type DNA-binding domain | 4 |
| 98455 | ace3 | 6 |
| 64599 | dim2 (DNMT) | 8 |
| 67752 | Fork head transcription factor | 10 |
| 128408 | HMG-box | 12 |
| 127621 | RNA polymerase sigma factor 54 interaction domain | 14 |
| 136173 | RNA polymerase sigma factor 54 interaction domain | 16 |
| 101389 | RNA-induced silencing complex, nuclease component | 18 |
| | Tudor-SN | |
| 130795 | Signal transduction response regulator, pH-responsive, | 20 |
| | Pall/Rim9 | |
| 23472 | Trp repressor binding protein-like | 22 |
| 7294 | Zn(2)-C6 fungal-type DNA-binding domain | 24 |
| 23425 | Zn(2)-C6 fungal-type DNA-binding domain | 26 |
| 52813 | Zn(2)-C6 fungal-type DNA-binding domain | 28 |
| 74630 | Zn(2)-C6 fungal-type DNA-binding domain | 30 |
| 76382 | Zn(2)-C6 fungal-type DNA-binding domain | 32 |
| 77124 | Zn(2)-C6 fungal-type DNA-binding domain | 34 |
| 78902 | Zn(2)-C6 fungal-type DNA-binding domain | 36 |
| 85090 | Zn(2)-C6 fungal-type DNA-binding domain | 38 |
| 91315 | Zn(2)-C6 fungal-type DNA-binding domain | 40 |
| 97880 | Zn(2)-C6 fungal-type DNA-binding domain | 42 |
| 100825 | Zn(2)-C6 fungal-type DNA-binding domain | 44 |
| 103144 | Zn(2)-C6 fungal-type DNA-binding domain | 46 |
| 103464 | Zn(2)-C6 fungal-type DNA-binding domain | 48 |
| 105117 | Zn(2)-C6 fungal-type DNA-binding domain | 50 |
| 109343 | Zn(2)-C6 fungal-type DNA-binding domain | 52 |
| 132448 | Zn(2)-C6 fungal-type DNA-binding domain | 54 |
| 133005 | Zn(2)-C6 fungal-type DNA-binding domain | 56 |
| 37062 | Zn(2)-C6 fungal-type DNA-binding domain | 124 |

### Example 6: Construction of Trichoderma reesei Transcription Factor 70883 Gene Deletion Plasmid pSMai320

Plasmid pSMai320 was constructed to delete the transcription factor (TF) 70883 gene (SEQ ID NO: 3 for the DNA sequence and SEQ ID NO: 4 for the deduced amino acid sequence) in Trichoderma reesei AgJg216-2B51. To construct a T. reesei TF 70883 gene deletion cassette, a PCR product (DNA fragment 1) containing a 2060 bp fragment of the upstream non-coding region of the T. reesei TF 70883 gene was PCR amplified using primers 1225696 and 1225697 shown below.

Forward primer 1225696:
(SEQ ID NO: 59)
GAGTCGACCTGCAGGCATGCGTTTAAACTTGGCCACCTA
CACTGCTACTA
Reverse primer 1225697:
(SEQ ID NO: 60)
CGTGAAGCCGTTTAAATGAAACTAGCTCCAGATGGAAATATAC

The amplification reaction was composed of approximately 180 ng of T. reesei BTR213 genomic DNA, 10 mM dNTPs, 50 μmol of forward primer, 50 μmol of reverse primer, 1× PHUSION® HF buffer (Thermo Fisher Scientific), and 2 units of PHUSION® Hot Start DNA polymerase (Thermo Fisher Scientific) in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 2,129 bp PCR fragment was purified by 0.8% agarose gel electrophoresis using TAE buffer, excised from the gel, and extracted using a NUCLEOSPIN® Gel and PCR Clean-up Kit (Macherey-Nagel).

A PCR product (DNA fragment 2) containing an E. coli hygromycin phosphotransferase gene (hpt) selection marker and the human Herpes simplex virus type 1 thymidine kinase gene (tk) selection marker cassette was PCR amplified using primers 1225698 and 1225699 shown below.

Forward primer 1225698:
(SEQ ID NO: 61)
TATTTCCATCTGGAGCTAGTTTCATTTAAACGGCTTCACGGG
Reverse primer 1225699:
(SEQ ID NO: 62)
TCGTTCGAAATTTTCTTCTAGAGAGTTCAAGGAAGAAACAGTGC

The amplification reaction was composed of approximately 10 ng of pJfyS1579-41-11 (US 20110223671 A1), 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 4,449 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 3) containing a 235 bp of the upstream non-coding region of the T. reesei TF 70883 gene, which will serve as a direct repeat region, was PCR amplified using primers 1225700 and 1225701 shown below.

Forward primer 1225700:
(SEQ ID NO: 63)
TGTTTCTTCCTTGAACTCTCTAGAAGAAAATTTCGAACGAACCG
Reverse primer 1225701:
(SEQ ID NO: 64)
GAAGAATCGACTGGCTGCCTACTAGCTCCAGATGGAAATATACT

The amplification reaction was composed of approximately 180 ng of T. reesei BTR213 genomic DNA, 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 323 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 4) containing a 2060 bp fragment of the downstream non-coding region of the T. reesei TF 70883 gene was PCR amplified using primers 1225702 and 1225703 shown below.

Forward primer 1225702:
(SEQ ID NO: 65)
TATTTCCATCTGGAGCTAGTAGGCAGCCAGTCGATTCTTCTT
Reverse primer 1225703:
(SEQ ID NO: 66)
GCTATGACCATGATTACGCCGTTTAAACCGTCCAGATAATGCGCACGC

The amplification reaction was composed of approximately 180 ng of T. reesei BTR213 genomic DNA, 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 2126 bp PCR fragment was purified by 0.8% agarose gel electrophoresis using TAE buffer, excised from the gel, and extracted using a NUCLEOSPIN® Gel and PCR Clean-up Kit.

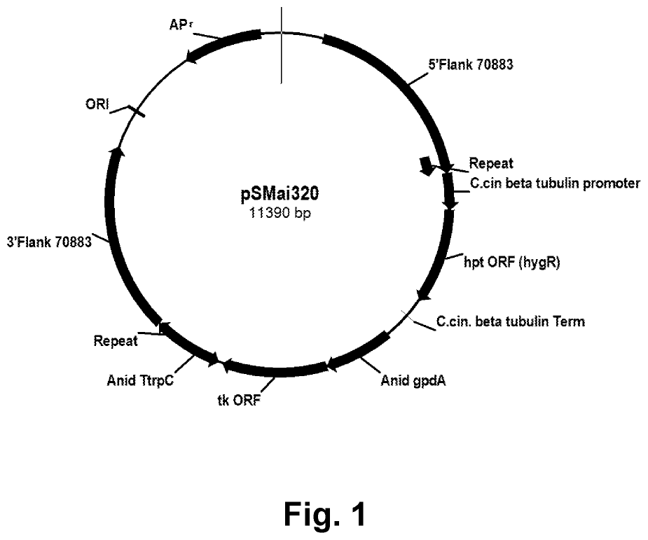

Plasmid pUC19 (New England BioLabs Inc.) was digested with Hind III and purified by 0.8% agarose gel electrophoresis using TAE buffer, where a 2,686 bp fragment was excised from the gel and extracted using a NUCLEOSPIN® Gel and PCR Clean-up Kit. The 2,686 bp fragment was assembled with the four PCR products (DNA fragments 1, 2, 3, and 4) described above using a NEBUILDER® HiFi DNA Assembly Cloning Kit (New England Biolabs Inc.) in a total volume of 20 μl composed of 1× NEBUILDER® HiFi Assembly Master Mix (New England Biolabs Inc.), and 0.05 μmol of each PCR product. The reaction was incubated at 50° C. for 60 minutes and then placed on ice. Two μl of the reaction were used to transform 50 μl of STELLAR™ chemically competent E. coli cells (Clontech Laboratories). The cells were heat shocked at 42° C. for 45 seconds and then 450 μl of SOC medium, pre-heated to 42° C., were added. The cells were incubated at 37° C. with shaking at 200 rpm for 60 minutes and then spread onto a 150 mm diameter 2XYT+Amp plate and incubated at 37° C. overnight. The resulting E. coli transformants were individually inoculated into 3 ml of LB+Amp medium in 14 ml round-bottom polypropylene tubes and incubated at 37° C. overnight with shaking at 200 rpm. Plasmid DNA was isolated using a BIOROBOT® 9600 (QIAGEN Inc.) and screened for proper insertion of the fragments by digestion with Nco I. A plasmid yielding the desired band sizes (6905 bp+4485 bp) was confirmed by DNA sequencing with a Model 377 XL Automated DNA Sequencer (Applied Biosystems Inc.) using dye-terminator chemistry (Giesecke et al., 1992, J. Virol. Methods 38(1): 47-60). One plasmid containing the insert with no PCR errors was identified and designated pSMai320 (FIG. 1).

### Example 7: Construction of Trichoderma reesei Transcription Factor (TF) 92949 Gene Deletion Plasmid pSMai321

Plasmid pSMai321 was constructed to delete the transcription factor (TF) 92949 gene (SEQ ID NO: 1 for the DNA sequence and SEQ ID NO: 2 for the deduced amino acid sequence) in Trichoderma reesei AgJg216-2B51. To construct a T. reesei TF 92949 gene deletion cassette, a PCR product (DNA fragment 1) containing a 2060 bp fragment of the upstream non-coding region of the T. reesei TF 92949 gene was PCR amplified using primers 1225708 and 1225709 shown below.

Forward primer 1225708:
(SEQ ID NO: 67)
GAGTCGACCTGCAGGCATGCGTTTAAACACACACAGGGGTACCGTTTC
Reverse primer 1225709:
(SEQ ID NO: 68)
CGTGAAGCCGTTTAAATGAAGTTGACGGTTGAGCAGAAAACGC

The amplification reaction was composed of approximately 180 ng of T. reesei BTR213 genomic DNA, 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 2,151 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 2) containing an E. coli hygromycin phosphotransferase (hpt) selection marker and the human Herpes simplex virus type 1 thymidine kinase gene (HSV-1 tk) selection marker cassette was PCR amplified using primers 1225710 and 1225711 shown below.

Forward primer 1225710:
(SEQ ID NO: 69)
TTTTCTGCTCAACCGTCAACTTCATTTAAACGGCTTCACGGGC
Reverse primer 1225711:
(SEQ ID NO: 70)
GAGTGGTGGGTTTGGTTTGCGAGAGTTCAAGGAAGAAACAGTGC

The amplification reaction was composed of approximately 10 ng of pJfyS1579-41-11 (US 20110223671), 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 4,450 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 3) containing a 235 bp of the upstream non-coding region of the T. reesei TF 92949 gene, which will serve as a direct repeat region, was PCR amplified using primers 1225712 and 1225713 shown below.

Forward primer 1225712:
(SEQ ID NO: 71)
TGTTTCTTCCTTGAACTCTCGCAAACCAAACCCACCACTCTAC
Reverse primer 1225713:
(SEQ ID NO: 72)
GATCAGTTCGGATACGCGCTGTTGACGGTTGAGCAGAAAACG

The amplification reaction was composed of approximately 180 ng of T. reesei BTR213 genomic DNA, 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 320 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 4) containing a 2065 bp fragment of the downstream non-coding region of the T. reesei TF 92949 gene was PCR amplified using primers 1225714 and 1225715 shown below.

Forward primer 1225714:
(SEQ ID NO: 73)
TTTTCTGCTCAACCGTCAACAGCGCGTATCCGAACTGATCTA
Reverse primer 1225715:
(SEQ ID NO: 74)
GCTATGACCATGATTACGCCGTTTAAACCTACCTGTCGAAGAA
ATAAAAGAGG

The amplification reaction was composed of approximately 180 ng of T. reesei BTR213 genomic DNA, 10 mM dNTPs, 50 μmol of forward primer, 50 μmol of reverse primer, 1× PHUSION® HF buffer, and 2 units of PHUSION® Hot Start DNA polymerase in a final volume of 50 μl. The reaction was incubated in a thermocycler programmed for 1 cycle at 98° C. for 3 minutes; 30 cycles each at 98° C. for 10 seconds, 58° C. for 30 seconds, and 72° C. for 2.5 minutes; 1 cycle at 72° C. for 10 minutes; and a 10° C. hold. The resulting 2,155 bp PCR fragment was purified by 0.8% agarose gel electrophoresis using TAE buffer, excised from the gel, and extracted using a NUCLEOSPIN® Gel and PCR Clean-up Kit.

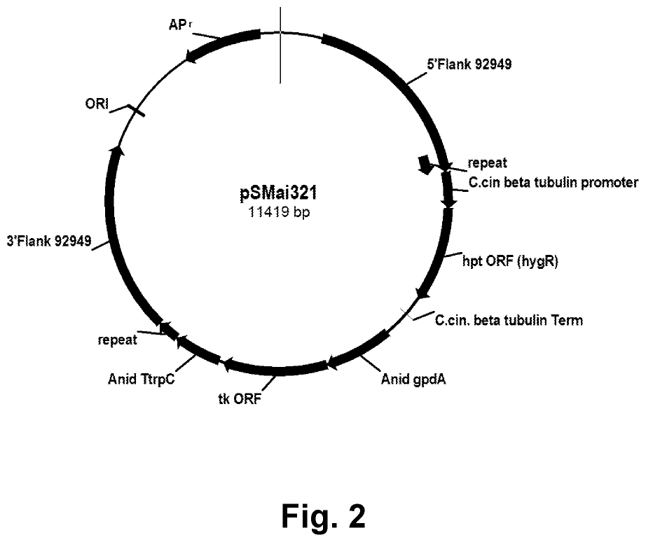

Plasmid pUC19 was digested with Hind III and purified by 0.8% agarose gel electrophoresis using TAE buffer, where a 2,686 bp fragment was excised from the gel and extracted using a NUCLEOSPIN® Gel and PCR Clean-up Kit. The 2,686 bp fragment was assembled with the four PCR products (DNA fragments 1, 2, 3, and 4) described above using a NEBUILDER® HiFi DNA Assembly Cloning Kit in a total volume of 20 μl composed of 1× NEBUILDER® HiFi Assembly Master Mix and 0.05 μmol of each PCR product. The reaction was incubated at 50° C. for 60 minutes and then placed on ice. Two μl of the reaction were used to transform 50 μl of STELLAR™ chemically competent E. coli cells. The cells were heat shocked at 42° C. for 45 seconds and then 450 μl of SOC medium, pre-heated to 42° C., were added. The cells were incubated at 37° C. with shaking at 200 rpm for 60 minutes and then spread onto a 150 mm diameter 2XYT+Amp plate and incubated at 37° C. overnight. The resulting E. coli transformants were individually inoculated into 3 ml of LB+Amp medium in 14 ml round-bottom polypropylene tubes and incubated at 37° C. overnight with shaking at 200 rpm. Plasmid DNA was isolated using a BIOROBOT® 9600 and screened for proper insertion of the fragments by digestion with Nco I. A plasmid yielding the desired band sizes (7422 bp and 3997 bp) was confirmed by DNA sequencing with a Model 377 XL Automated DNA Sequencer using dye-terminator chemistry (Giesecke et al., 1992, supra). One plasmid containing the insert with no PCR errors was identified and designated pSMai321 (FIG. 2).

### Example 8: Generation of Single Transcription Factor (TF) 70883 Deletion Trichoderma reesei Strain QMJ1122-16B4-4

Protoplasts of Trichoderma reesei strain AgJg216-2B51 were generated and transformed according to Example 2 to delete the transcription factor (TF) 70883 gene. Protoplasts were transferred to 15 round-bottom polypropylene tubes and transformed with 3 μg of Pme I-linearized and gel purified pSMai320 (Example 6). Seven transformants were selected on PDA plates containing hygromycin B. Each of the transformants were transferred to a PDA plate and incubated for 5 days at 30° C. to generate spores.

The transformants of T. reesei AgJg216-2B51 were screened by a fungal spore PCR method using a PHIRE™ Plant Direct PCR Kit (Thermo Fisher Scientific Inc.) for the presence of the pSMai320 deletion vector at the TF 70883 locus. A small amount of spores from each transformant was suspended in 20 μl of Dilution buffer (PHIRE™ Plant Direct PCR Kit). The spore suspensions were used as templates in the PCRs to screen for the TF 70883 gene deletion. Each reaction was composed of 0.5 μl of the spore suspension, 10 μmol of each primer shown below (2-3 primers for each PCR), 5 μl of 2× PHIRE™ Plant PCR Buffer, and 0.2 μl of PHIRE™ Hot Start II DNA Polymerase in a 10 μl reaction.

### PCR Screen of 5′ End of TF 70883 Locus:

Forward primer 1225977:
(SEQ ID NO: 75)
TGACCGGGCAGGGGATCGCC
Reverse primer 1225978:
(SEQ ID NO: 76)
CTGGGGCGTCAAGGGACCTGAATG
Reverse primer 1219245:
(SEQ ID NO: 77)
CTACATCGAAGCTGAAAGCACGAGA

### PCR Screen of 3′ End of TF 70883 Locus:

Forward primer 1213333:
(SEQ ID NO: 78)
GGGACGCCCTGCTGCAACTTACC
Reverse primer 1225979:
(SEQ ID NO: 79)
CGCCCTTCGACGAGTCGGCAC

The reactions were incubated in a thermocycler programmed for 1 cycle at 98° C. for 5 minutes; 40 cycles each at 98° C. for 10 seconds, 65° C. for 10 seconds, and 72° C. for 90 seconds; 1 cycle at 72° C. for 1 minute; and a 4° C. hold. The completed PCRs were analyzed by 1.5% agarose gel electrophoresis using TAE buffer. Transformants having correct targeting of pSMai320 DNA to the TF 70883 locus produced a 3 kb PCR fragment.

One transformant, T. reesei QMJ1122-16, that produced the correct PCR fragment was chosen for single spore isolation. A small number of spores from a 6 day old PDA plate were collected in 5 ml of 0.01% TWEEN® 20 solution. A 2 μl aliquot of the spore solution was mixed with 100 μl of a 0.01% TWEEN® 20 solution and spread onto a 150 mm PDA plate supplemented with 1 M sucrose. The plate was incubated at 30° C. for 2-3 days. Single colonies were transferred onto PDA plates and incubated at 30° C. for 5-7 days. Fungal spore PCR was utilized to identify spore isolates with correct targeting to the TF 70883 locus as described in Example 8.

The deletion construct pSMai320 contains the positively-selectable hygromycin phosphoryl transferase (hpt) gene and the negatively-selectable thymidine kinase (tk) gene, flanked by direct repeats. The direct repeats were inserted to facilitate the excision of the hpt and tk selectable markers and generate a marker-free strain for use as a host to delete a second transcription factor gene.

Spores from T. reesei QMJ1122-16 were spread onto Trichoderma Minimal Medium plates containing 1.5 μM 5-fluoro-2′-deoxyuridine (FdU) at concentrations of 1×10⁴, 1×10⁵ and 1×10⁶ and incubated at 30° C. for 5 days. Twenty isolates were sub-cultured onto PDA plates and incubated at 30° C. for 4 days. All 20 isolates were then screened for the absence of the hpt and tk selectable marker genes by fungal spore PCR method using a PHIRE™ Plant Direct PCR Kit. A small amount of spores from each isolate was suspended in 20 μl of Dilution buffer (PHIRE™ Plant Direct PCR Kit). The spore suspensions were used as templates in the PCRs to screen for the absence of the hpt and tk selection marker genes at the TF 70883 locus. Each reaction was composed of 0.5 μl of the spore suspension, 10 μmol of each primer shown below (3 primers), 5 μl of 2× PHIRE™ Plant PCR Buffer, and 0.2 μl of PHIRE™ Hot Start II DNA Polymerase in a 10 μl reaction.

Forward primer 1225977:
(SEQ ID NO: 80)
TGACCGGGCAGGGGATCGCC
Reverse primer 1225979:
(SEQ ID NO: 81)
CGCCCTTCGACGAGTCGGCAC
Reverse primer 1219245:
(SEQ ID NO: 82)
CTACATCGAAGCTGAAAGCACGAGA

The reactions were incubated in a thermocycler programmed for 1 cycle at 98° C. for 5 minutes; 40 cycles each at 98° C. for 10 seconds, 65° C. for 10 seconds, and 72° C. for 90 seconds; 1 cycle at 72° C. for 1 minute; and a 4° C. hold. The completed PCRs were analyzed by 1.5% agarose gel electrophoresis using TAE buffer. Isolates with the hpt and tk selection marker genes excised produced a 4.3 kb PCR fragment.

Isolates that showed correct excision of the marker genes underwent another round of single isolation followed by fungal spore PCR as described above. Genomic DNA was prepared as described in Example 1 and sequenced using 2×150 bp chemistry in NEXTSEQ™ 500. Sequencing identified transformant T. reesei QMJ1122-16B4-4 as containing the TF 70883 gene deletion and absence of the hpt and tk selection marker genes.

### Example 9: Generation of Single Transcription Factor (TF) 92949 Deletion Trichoderma reesei Strain QMJI121-8D7-3

Protoplasts of Trichoderma reesei strain AgJg216-2B51 were generated and transformed according to Example 2 to delete the transcription factor (TF) 92949 gene. Protoplasts were transferred to 15 round-bottom polypropylene tubes and transformed with 3 μg of Pme I-linearized and gel purified pSMai321 (Example 7). Twelve transformants were selected on PDA plates containing hygromycin B. Each of the transformants were transferred to a PDA plate and incubated for 5 days at 30° C. to generate spores. The transformants of T. reesei AgJg216-2B51 were screened by a fungal spore PCR method using a PHIRE™ Plant Direct PCR Kit for the presence of the pSMai321 deletion vector at the TF 92949 gene locus. A small amount of spores from each transformant was suspended in 20 μl of Dilution buffer (PHIRE™ Plant Direct PCR Kit). The spore suspensions were used as templates in the PCRs to screen for the TF 92949 gene deletion. Each reaction was composed of 0.5 μl of the spore suspension, 10 μmol of each primer shown below (2-3 primers for each PCR), 5 μl of 2× PHIRE™ Plant PCR Buffer, and 0.2 μl of PHIRE™ Hot Start II DNA Polymerase in a 10 μl reaction.

### PCR Screen of 5′ End of TF 92949 Locus:

Forward primer 1225980:
(SEQ ID NO: 83)
TGCCCTGGTTTCGCGCATACGG
Reverse primer 1225981:
(SEQ ID NO: 84)
ACGGATAGGAGCAGCAAAGCAAAGGC
Reverse primer 1219245:
(SEQ ID NO: 85)
CTACATCGAAGCTGAAAGCACGAGA

### PCR Screen of 3′ End of TF 92949 Locus:

Forward primer 1213333:
(SEQ ID NO: 86)
GGGACGCCCTGCTGCAACTTACC
Reverse primer 1225982:
(SEQ ID NO: 87)
GAGACGAGACTGGAGTCGTTGCCGC

The reactions were incubated in a thermocycler programmed for 1 cycle at 98° C. for 5 minutes; 40 cycles each at 98° C. for 10 seconds, 65° C. for 10 seconds, and 72° C. for 90 seconds; 1 cycle at 72° C. for 1 minute; and a 4° C. hold. The completed PCRs were analyzed by 1.5% agarose gel electrophoresis using TAE buffer. Transformants having correct targeting of pSMai321 DNA to the TF 92949 gene locus produced a 3 kb PCR fragment.

One transformant, T. reesei QMJ1121-8, that produced the correct PCR fragment was chosen for single spore isolation. A small number of spores from a 6 day old PDA plate were collected in 5 ml of 0.01% TWEEN® 20 solution. A 2 μl aliquot of the spore solution was mixed with 100 μl of a 0.01% TWEEN® 20 solution and spread onto a 150 mm PDA plate supplemented with 1 M sucrose. The plate was incubated at 30° C. for 2-3 days. Single colonies were transferred onto PDA plates and incubated at 30° C. for 5-7 days. Fungal spore PCR was utilized to identify spore isolates with correct targeting to the TF 92949 locus.

The deletion construct pSMai321 contains the positively-selectable hygromycin phosphoryl transferase (hpt) gene and the negatively-selectable thymidine kinase (tk) gene, flanked by direct repeats. The direct repeats were inserted to facilitate the excision of the hpt and tk selectable marker genes and generate a marker-free strain for use as a host to delete a second transcription factor gene.

Spores from T. reesei QMJ1121-8 were spread onto Trichoderma Minimal Medium plates containing 1.5 μM 5-fluoro-2′-deoxyuridine (FdU) at concentrations of 1×10⁴, 1×10⁵ and 1×10⁶ and incubated at 30° C. for 5 days. Twenty isolates were sub-cultured onto PDA plates and incubated at 30° C. for 4 days. All 20 isolates were then screened for the absence of the hpt and tk selection marker genes by fungal spore PCR method using a PHIRE™ Plant Direct PCR Kit. A small amount of spores from each isolates was suspended in 20 μl of Dilution buffer (PHIRE™ Plant Direct PCR Kit). The spore suspensions were used as templates in the PCR to screen for the absence of the hpt and tk selection marker genes at the TF 92949 locus. Each reaction was composed of 0.5 μl of the spore suspension, 10 μmol of each primer shown below (3 primers), 5 μl of 2× PHIRE™ Plant PCR Buffer, and 0.2 μl of PHIRE™ Hot Start II DNA Polymerase in a 10 μl reaction.

Forward primer 1225980:
(SEQ ID NO: 88)
TGCCCTGGTTTCGCGCATACGG
Reverse primer 1225982:
(SEQ ID NO: 89)
GAGACGAGACTGGAGTCGTTGCCGC
Reverse primer 1219245:
(SEQ ID NO: 90)
CTACATCGAAGCTGAAAGCACGAGA

The reactions were incubated in a thermocycler programmed for 1 cycle at 98° C. for 5 minutes; 40 cycles each at 98° C. for 10 seconds, 65° C. for 10 seconds, and 72° C. for 90 seconds; 1 cycle at 72° C. for 1 minute; and a 4° C. hold. The completed PCRs were analyzed by 1.5% agarose gel electrophoresis using TAE buffer. Isolates with the hpt and tk selection marker genes excised produced a 4.4 kb PCR fragment.

Isolates that showed correct excision of the marker genes underwent another round of single isolation followed by fungal spore PCR as described above. Genomic DNA was prepared as described in Example 1 and sequenced using 2×150 bp chemistry in NEXTSEQ™ 500. Sequencing identified transformant T. reesei QMJ1121-8D7-3 as containing the TF 92949 gene deletion and absence of the hpt and tk selection marker genes.

### Example 10: Generation of Double Transcription Factor (TF) Deletion Trichoderma reesei Strain SMai321-1C2-1

Protoplasts of Trichoderma reesei single transcription factor TF 70883 deletion strain QMJ1122-16B4-4 were generated and transformed according to Example 2 to delete the second transcription factor (TF) 92949 gene. Protoplasts were transferred to ten round-bottom polypropylene tubes and transformed with 4 μg of Pme I-linearized and gel purified pSMai321 (Example 7). Twenty transformants were selected on PDA plates containing hygromycin B. Each of the transformants were transferred to a PDA plate and incubated for 5 days at 30° C. to generate spores. The transformants of T. reesei QMJ1122-16B4-4 were screened by a fungal spore PCR method using a PHIRE™ Plant Direct PCR Kit for the presence of the pSMai321 deletion vector at the TF 92949 gene locus. A small amount of spores from each transformant was suspended in 20 μl of Dilution buffer (PHIRE™ Plant Direct PCR Kit). The spore suspensions were used as templates in the PCR to screen for the TF 92949 gene deletion. Each reaction was composed of 0.5 μl of the spore suspension, 10 μmol of each primer shown below (2-3 primers for each PCR), 5 μl of 2× PHIRE™ Plant PCR Buffer, and 0.2 μl of PHIRE™ Hot Start II DNA Polymerase in a 10 μl reaction.

### PCR Screen of 5′ End of TF 92949 Locus:

Forward primer 1225980:
(SEQ ID NO: 91)
TGCCCTGGTTTCGCGCATACGG
Reverse primer 1225981:
(SEQ ID NO: 92)
ACGGATAGGAGCAGCAAAGCAAAGGC
Reverse primer 1219245:
(SEQ ID NO: 93)
CTACATCGAAGCTGAAAGCACGAGA

### PCR Screen of 3′ End of TF 92949 Locus:

Forward primer 1213333:
(SEQ ID NO: 94)
GGGACGCCCTGCTGCAACTTACC
Reverse primer 1225982:
(SEQ ID NO: 95)
GAGACGAGACTGGAGTCGTTGCCGC

The reactions were incubated in a thermocycler programmed for 1 cycle at 98° C. for 5 minutes; 40 cycles each at 98° C. for 10 seconds, 65° C. for 10 seconds, and 72° C. for 90 seconds; 1 cycle at 72° C. for 1 minute; and a 4° C. hold. The completed PCRs were analyzed by 1.5% agarose gel electrophoresis using TAE buffer. Transformants having correct targeting of pSMai321 DNA to the TF 92949 gene locus produced a 3 kb PCR fragment.

Three transformants (T. reesei SMai321-1, SMai321-3, and SMai321-14) that produced the correct PCR fragment were chosen for single spore isolation. A small number of spores from a 6 day old PDA plate for each transformant were collected in 5 ml of 0.01% TWEEN® 20 solution. A 2 μl aliquot of each spore solution was mixed with 100 μl of a 0.01% TWEEN® 20 solution and spread onto a 150 mm PDA plate supplemented with 1 M sucrose. The plates were incubated at 30° C. for 2-3 days. Single colonies were transferred onto PDA plates and incubated at 30° C. for 5-7 days. Fungal spore PCR was utilized to identify spore isolates with correct targeting to TF 92949 locus as described above.

The deletion construct pSMai321 contains the positively-selectable hygromycin phosphoryl transferase (hpt) gene and the negatively-selectable thymidine kinase (tk) gene, flanked by direct repeats. The direct repeats were inserted to facilitate the excision of the hpt and tk selectable markers and generate a marker-free strain.

Spores from T. reesei SMai321-1, SMai321-3, and SMai321-14 were spread onto Trichoderma minimal medium plates containing 1.5 μM 5-fluoro-2′-deoxyuridine (FdU) at concentrations of 1×10⁴, 1×10⁵ and 1×10⁶ and incubated at 30° C. Thirty isolates were sub-cultured onto PDA plates and incubated at 30° C. for 4 days. All 30 isolates were then screened for the absence of the hpt and tk selection marker genes by fungal spore PCR method using a PHIRE™ Plant Direct PCR Kit. A small amount of spores from each isolate was suspended in 20 μl of Dilution buffer (PHIRE™ Plant Direct PCR Kit). The spore suspensions were used as templates in the PCRs to screen for the absence of the hpt and tk selection marker genes at the TF 92949 locus. Each reaction was composed of 0.5 μl of the spore suspension, 10 μmol of each primer shown below (3 primers), 5 μl of 2× PHIRE™ Plant PCR Buffer, and 0.2 μl of PHIRE™ Hot Start II DNA Polymerase in a 10 μl reaction.

Forward primer 1225980:
(SEQ ID NO: 96)
TGCCCTGGTTTCGCGCATACGG
Reverse primer 1225982:
(SEQ ID NO: 97)
GAGACGAGACTGGAGTCGTTGCCGC
Reverse primer 1219245:
(SEQ ID NO: 98)
CTACATCGAAGCTGAAAGCACGAGA

The reactions were incubated in a thermocycler programmed for 1 cycle at 98° C. for 5 minutes; 40 cycles each at 98° C. for 10 seconds, 65° C. for 10 seconds, and 72° C. for 90 seconds; 1 cycle at 72° C. for 1 minute; and a 4° C. hold. The completed PCRs were analyzed by 1.5% agarose gel electrophoresis using TAE buffer. Isolates with the hpt and tk selection marker genes excised produced a 4.4 kb PCR fragment.

Isolates that showed correct excision of the marker genes underwent another round of single isolation followed by fungal spore PCR as described above. Genomic DNA was prepared as described in Example 1 and sequenced using 2×150 bp chemistry in NEXTSEQ™ 500. Sequencing identified transformant T. reesei SMai321-1C2-1 as containing the TF 92949 gene deletion and absence of the hpt and tk selection marker genes.

### Example 11: Lysozyme Activity Assay

Whole broth from a 2 L fermentation was mixed for roughly 2 hours in a rotisserie mixer at 30° C. After whole broth mixing, all samples were diluted 100× in 40% urea, then mixed for approximately 2 hours using the rotisserie mixer. The 100× diluted samples were diluted 1,000,000× in sample buffer (0.1 M Tris, 0.1 M NaCl, 0.01% TRITON® X-100 buffer pH 7.5) by 10-fold serial dilutions followed by a series dilution from 0-fold to ⅓-fold to 1/9-fold of the diluted sample. A lysozyme standard was diluted from 0.05 LSU(F)/ml concentration to a 0.002 LSU(F)/ml concentration in the sample buffer (0.1 M Tris, 0.1M NaCl, 0.01% Triton X-100 buffer pH 7.5). A total of 50 μl of each dilution including the standard was transferred to a 96-well flat bottom plate. Fifty μl of a 25 μg/ml fluorescein-conjugated cell wall substrate solution was added to each well and then incubated at ambient temperature for 45 minutes. During the incubation, the rate of the reaction was monitored at 485 nm (excitation)/528 nm (emission) for the 96-well plate at 15-minute intervals on a SPECTRAMAX® plate reader (Molecular Devices LLC). Sample concentrations were determined by extrapolation from the generated standard curve.

### Example 12: Comparing Acremonium alcalophilum GH25 Lysozyme Productivity in Trichoderma reesei AgJg216-2B51, Single Transcription Factor 70883 Deletion Strain QMJ1122-16B4-4, and Single Transcription Factor 92949 Deletion Strain QMJI121-8D7-3 in 2 Liter Fermentations

Single transcription factor 70883 deletion strain T. reesei QMJ1122-16B4-4 (Example 8), single transcription factor 92949 deletion strain T. reesei QMJ1121-8D7-3 (Example 9), and host strain T. reesei AgJg216-2B51 were evaluated in 2 liter fermentations. Each strain was grown on a PDA agar plate for 4-7 days at 30° C. Three 500 ml shake flasks each containing 100 ml of Shake Flask Medium were inoculated with two plugs from a PDA agar plate. The shake flasks were incubated at 28° C. for 48 hours on an orbital shaker at 250 rpm. The cultures were used as seed for fermentation.

A total of 160 ml of each seed culture was used to inoculate 3-liter glass jacketed fermentors (Applikon Biotechnology) containing 1.6 liters of Fermentation Batch Medium. The fermentors were maintained at a temperature of 28° C. and pH was controlled using an Applikon 1030 control system (Applikon Biotechnology) to a set-point of 3.5+/−0.1. Air was added to the vessel at a rate of 2.5 L/minute and the broth was agitated by Rushton impeller rotating at 1100 rpm. Fermentation feed medium composed of dextrose and phosphoric acid was dosed at a rate of 0 to 10 g/L/hour for a period of 165 hours. Samples were taken on days 3, 4, 5, 6, and 7 of the fermentation run. The whole broths were stored at 4° C.

The Acremonium alcalophilum GH25 lysozyme expression level was determined on the whole broth samples as described in Example 11. A 1.36× increase in A. alcalophilum lysozyme activity in the strain with the TF70883 deletion containing 4-copies of the A. alcalophilum lysozyme gene was observed compared to the host strain T. reesei AgJg216-2651 (Table 2). Similarly, a 1.27× increase in A. alcalophilum lysozyme activity in the strain with the TF92949 deletion containing 4-copies of the A. alcalophilum lysozyme gene was observed compared to the host strain T. reesei AgJg216-2B51 (Table 2).

TABLE 2
Comparing relative lysozyme activity in Trichoderma reesei host and
single transcription factor deletion strains at 7 days

| | Transcription Factor | Relative lysozyme |
|---|---|---|
| Strain | Gene Deletion | Activity |
| AgJg216-2651 (host) | — | 1.00 |
| QMJI122-1664-4 | TF70883 | 1.36 |
| QMJI121-8D7-3 | TF92949 | 1.27 |

### Example 13: Comparing Acremonium alcalophilum GH25 Lysozyme Productivity in Trichoderma reesei Single Transcription Factor 70883 Deletion Strain QMJ1122-16B4-4 and Trichoderma reesei Double Transcription Factor Deletion Strain SMai321-1C2-1 in 2 Liter Fermentations

Single transcription factor 70883 deletion strain T. reesei QMJ1122-16B4-4 and double transcription factors deletion strain T. reesei SMai321-1C2-1 were evaluated in 2 liter fermentations. Each strain was grown on a PDA agar plate for 4-7 days at 30° C. Three 500 ml shake flasks each containing 100 ml of Shake Flask Medium were inoculated with two plugs from a PDA agar plate. The shake flasks were incubated at 28° C. for 48 hours on an orbital shaker at 250 rpm. The cultures were used as seed for fermentation.

A total of 160 ml of each seed culture was used to inoculate 3-liter glass jacketed fermentors (Applikon Biotechnology) containing 1.6 liters of Fermentation Batch Medium. The fermentors were maintained at a temperature of 28° C. and pH was controlled using an Applikon 1030 control system to a set-point of 3.5+/−0.1. Air was added to the vessel at a rate of 2.5 L/min and the broth was agitated by Rushton impeller rotating at 1100 rpm. Fermentation feed medium composed of dextrose and phosphoric acid was dosed at a rate of 0 to 10 g/L/hour for a period of 165 hours. Samples were taken on days 3, 4, 5, 6, and 7 of the fermentation run. The whole broths were stored at 4° C.

The Acremonium alcalophilum GH25 lysozyme expression level was determined on the whole broth samples as described in Example 11. A 1.25× increase in A. alcalophilum lysozyme activity in the strain with two transcription factor gene deletions containing 4-copies of the A. alcalophilum lysozyme gene was observed compared to the single TF70883 transcription factor deletion strain, T. reesei QMJ1122-16B4-4 (Table 3).

TABLE 3
Comparing relative lysozyme activity in Trichoderma reesei single
transcription factor 70883 deletion and double transcription factor
deletion strains at 7 days

| | Transcription Factor | Relative lysozyme |
|---|---|---|
| Strain | Gene(s) Deletion | Activity |
| QMJI122-1664-4 | TF70883 | 1.00 |
| SMai321-1C2-1 | TF70883 & TF92949 | 1.25 |

### Example 14: CRISPR-MAD7 Backbone Plasmid pSMai322a

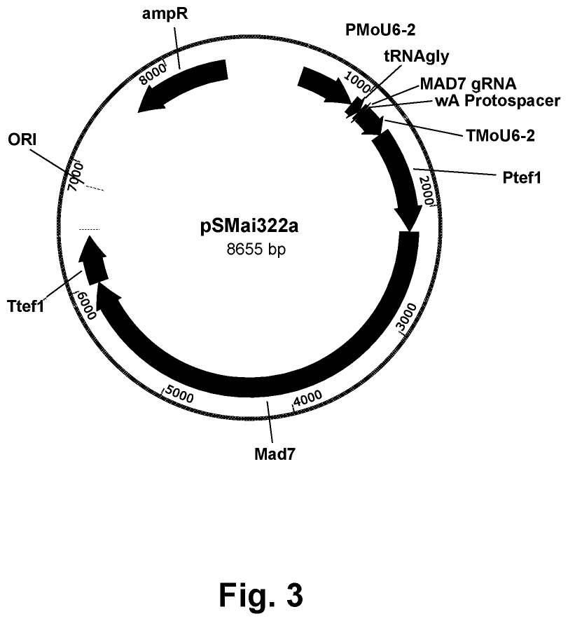

Plasmid pSMai322a (SEQ ID NO: 99, FIG. 3) is a CRISPR-MAD7 expression plasmid containing an Eubacterium rectale MAD7 gene codon optimized for Aspergillus oryzae with a SV40 NLS sequence under the transcriptional control of the Aspergillus nidulans tef1 promoter and terminator. In addition, it has a wA-sgRNA expression cassette, comprising a Magnaporthe oryzae U6-2 promoter, Aspergillus fumigatus tRNAgly(GCC)1-6 sequence, wA protospacer, Eubacterium rectale single guide RNA sequence, and Magnaporthe oryzae U6-2 terminator.

### Example 15: Construction of Plasmid pJFYS331 for CRISPR-MAD7 Mediated Simultaneous Gene Deletion of Transcription Factor Genes 92949 and 70883

Plasmid pJFYS331 was constructed for expression of MAD7 and single guide RNA to target the transcription factor genes 92949 and 70883, simultaneously, in Trichoderma reesei. The first step in the construction of plasmid pJFYS331 was to insert a 20 bp protospacer region targeting the T. reesei transcription factor gene 70883 into the MAD7 expression plasmid pSMai322a (Example 14). The MAD7 expression plasmid pSMai322a was linearized with Bgl II and purified by 1% agarose gel electrophoresis using TAE buffer, where an 8,655 bp fragment was excised from the gel and extracted using a NUCLEOSPIN® Gel Clean-up Kit. The 8,655 bp fragment was assembled with the single stranded oligonucleotide primer 1227488 shown below containing a 20 bp protospacer region targeting the T. reesei transcription factor gene 70883 and flanking homologous sequences for insertion into plasmid pSMai322a using a NEBUILDER® HiFi DNA Assembly Cloning Kit in a total volume of 10 μl composed of 1× NEBUILDER® HiFi Assembly Master Mix, 50 ng of linearized vector, and 0.05 μmol of oligonucleotide.

Primer 1227488:
(SEQ ID NO: 100)
TTTAATTTCTACTCTTGTAGATGCATTGTCAAAGCATCGCCCATTTTT
TTGGCTCTTGGGTTC

After incubating the mixture for 45 minutes at 50° C., 2 μl of the reaction were transformed into 50 μl of STELLAR™ chemically competent E. coli cells. The cells were heat shocked at 42° C. for 45 seconds after which 100 μl of SOC medium were added and the total volume was spread onto a 150 mm 2XYT+Amp plate and incubated at 37° C. overnight. The resulting E. coli transformants were individually inoculated into 3 ml of LB+Amp medium in 14 ml round-bottom polypropylene tubes and incubated at 37° C. overnight with shaking at 200 rpm. Plasmid DNA was isolated using a BIOROBOT® 9600. The insert was confirmed by DNA sequencing with a Model 377 XL Automated DNA Sequencer using dye-terminator chemistry. One plasmid was selected and designated pJFYS331a.

The second step in the construction of plasmid pJFYS331 was to insert a synthetic DNA encoding the Aspergillus fumigatus U6-3 promoter, the A. fumigatus tRNAgly sequence, a 20 bp protospacer region targeting the T. reesei transcription factor 92949 gene, the S. pyogenes single guide RNA sequence, the A. fumigatus U6-3 terminator, and flanking homologous sequences into plasmid pJFYS331a. A synthetic DNA sequence containing 879 bp was synthesized as a STRING™ DNA fragment (SEQ ID NO: 101) by GENEART® (ThermoFisher Scientific). The lyophilized DNA supplied by GENEART® containing the fragment was re-suspended in deionized water at a concentration of 30 ng/μl. Plasmid pJFSY331a was linearized with Pae I and purified by 1% agarose gel electrophoresis using TAE buffer, where a 9,494 bp fragment was excised from the gel and extracted using a NUCLEOSPIN® Gel Clean-up Kit. The 9,494 bp fragment was assembled with the STRING™ DNA fragment containing the elements described above using a NEBUILDER® HiFi DNA Assembly Cloning Kit in a total volume of 20 μl composed of 1× NEBUILDER® HiFi Assembly Master Mix, 100 ng of linearized vector, and 60 ng of STRING™ DNA fragment.

After incubating the mixture for 15 minutes at 50° C., 2 μl of the reaction were transformed into 50 μl of STELLAR™ chemically competent E. coli cells. The cells were heat shocked at 42° C. for 45 seconds after which 100 μl of SOC medium were added and the total volume was spread onto a 150 mm 2XYT+Amp plate and incubated at 37° C. overnight. The resulting E. coli transformants were individually inoculated into 3 ml of LB+Amp medium in 14 ml round-bottom polypropylene tubes and incubated at 37° C. overnight with shaking at 200 rpm. Plasmid DNA was isolated using a BIOROBOT® 9600. The insert was confirmed by DNA sequencing with a Model 377 XL Automated DNA Sequencer using dye-terminator chemistry. One plasmid was selected and designated pJFYS331.

### Example 16: Construction of Marker-Less Repair Plasmid pAMFS-TF92949D for Deletion of the Transcription Factor (TF) Gene 92949

In order to simultaneously target both transcription factor genes for deletion using CRISPR-MAD7 in a way that the selection markers can be removed following deletion, plasmid pAMFS-TF92949D, a hpt-tk selection marker-free version of pSMai321 (Example 7), was constructed. In the CRISPR-MAD7 multi-gene deletion scheme being used, it is desirable to only utilize the hpt and tk selection marker system from one plasmid pSMai320 will contain the hpt-tk selection markers). This is done to ensure that marker loop out need only occur from a single locus. Therefore, plasmid pAMFS-TF92949D was constructed to use in the multiplex scheme alongside pSMai320 for deletion of both transcription factor genes 92949 and 70883.

Plasmid pSMai321 (Example 7) was digested with Nco I and Xho I to generate linear DNA fragments of the following sizes: 5,669 bp, 1,753 bp, and 3,997 bp. The 5,669 bp fragment was purified by 0.8% agarose gel electrophoresis using TAE buffer, excised from the gel, and extracted using a NUCLEOSPIN® Gel Clean-up Kit. The resulting DNA was treated with DNA Pol I, Large (Klenow) fragment (New England Biolabs Inc.) in a 70 μl reaction containing 420 ng of the 5,669 bp fragment of pSMai321. The reaction was incubated at 25° C. for 15 minutes. The DNA was purified from this reaction mixture using a NUCLEOSPIN® PCR Clean-up Kit. The DNA was then ligated into a circular form using T4 DNA ligase (New England Biolabs Inc.) in a 20 μl reaction containing 60 ng of the DNA purified from the previous step. The reaction was incubated at 16° C. for approximately 18 hours.

Two μl of the reaction were used to transform 50 μl of STELLAR™ chemically competent E. coli cells. The cells were heat shocked at 42° C. for 45 seconds and then 450 μl of SOC medium, pre-heated to 42° C., were added. The cells were incubated at 37° C. with shaking at 200 rpm for 60 minutes and then spread onto a 150 mm diameter 2XYT+Amp plate and incubated at 37° C. overnight. The resulting E. coli transformants were individually inoculated into 3 ml of LB+Amp medium in 14 ml round-bottom polypropylene tubes and incubated at 37° C. overnight with shaking at 200 rpm. Plasmid DNA was isolated using a BIOROBOT® 9600. The plasmid was confirmed by DNA sequencing with a Model 377 XL Automated DNA Sequencer using dye-terminator chemistry (Giesecke et al., 1992, supra). One plasmid containing the insert with no PCR errors was identified and designated pAMFS-TF92949D.

### Example 17: Preparation of Linear Plasmid DNA for Transformation and Homologous Recombination Mediated Deletion of Transcription Factor Genes 70883 or 92949

A total of 100 μg of either plasmid pSMai320 (Example 6) or plasmid pSMai321 (Example 7) were combined in a 1.7 ml EPPENDORF™ tube with 25 μl of Bam HI and 100 μl of CUTSMART® buffer (New England BioLabs), and brought to a final volume of 1 ml with sterile water. The reactions were incubated at 37° C. for 3 hours. The DNA was purified using MINELUTE™ columns (QIAGEN Inc.), where each reaction was divided across ten columns and DNA eluted in 10 μl of sterile water per column. This yielded linear 11.390 kb DNA for pSMai320 and linear 11.419 kb DNA for pSMai321.

### Example 18: Generation of Single Transcription Factor (TF) 92949 Deletion Trichoderma reesei Cellulase Strain

Protoplasts of Trichoderma reesei cellulase strain GMer61-A19 were generated and transformed according to Example 2 to delete the transcription factor (TF) 92949 gene. Protoplasts were transferred to 15 round-bottom polypropylene tubes and transformed with 5 μg of Bam HI-linearized and gel purified pSMai321 (Example 7). Primary transformants were selected on PDA plates containing hygromycin B. Each of the transformants were transferred to a PDA plate and incubated for 5 days at 30° C. to generate spores.

To confirm that correct integration of the linearized pSMai321 DNA had occurred through homologous recombination, primary isolates were screened by fungal spore PCR using a PHIRE™ Plant Direct PCR Kit. A small number of spores from each isolate was resuspended in 20 μl of Dilution buffer. The spore suspensions were used as templates in the PCRs to screen for the integration of the linearized pSMai321. The PCRs were composed of 0.5 μl of the spore suspension, 50 μmol of each primer shown below, 5 ml of 2× PHIRE™ Plant PCR Buffer, and 0.2 ml of PHIRE™ Hot Start II DNA Polymerase in a 10 μl reaction.

Forward Primer 1227992:
(SEQ ID NO: 102)
GAATTCAAAAGCGCCAGTCACTGCGAG
Reverse Primer 1219245:
(SEQ ID NO: 103)
CTACATCGAAGCTGAAAGCACGAGA
Reverse Primer 1227993:
(SEQ ID NO: 104)
TCGTCGAGTCGAAGATGAGAGAGGATGG

The PCRs were performed in a thermocycler programmed for 1 cycle at 98° C. for 5 minutes; 40 cycles each at 98° C. for 10 seconds, 65° C. for 10 seconds, and 72° C. for 1 minute and 30 seconds; and 1 cycle at 72° C. for 1 minute. The completed PCRs were analyzed by 1% agarose gel electrophoresis using TAE buffer. Primary isolates with the correct diagnostic DNA band were selected for single spore purification.

Single spore isolation was performed once to insure the purity of the strains prior to removal of the hpt selection marker gene. A small number of spores from each isolate was suspended in 1 ml of sterile water. A 1 μl aliquot of each spore solution was mixed with 50 μl of sterile water and spread onto a 150 mm PDA plate supplemented with 1 M sucrose. The plates were incubated at 30° C. for 3-4 days.

Single colonies were transferred from the PDA-sucrose plates onto PDA plates and incubated at 30° C. for 5-7 days.

To confirm that the correct integration of the linearized pSMai321 DNA had occurred through homologous recombination, isolates were screened by fungal spore PCR using a PHIRE™ Plant Direct PCR Kit as described above. Isolates with the correct diagnostic DNA band were selected for selection marker loop out.

Spores were collected into sterile deionized water from the PDA plates that had been incubated at 30° C. for 5-7 days. The spores were counted and different amounts of spores (1×10⁷, 1×10⁶, 1×10⁵) were spread onto Trichoderma Minimal Medium plates containing 1.5 μM 5-fluoro-2′-deoxyuridine (FdU) and incubated at 30° C. for 5 days. Single colonies were transferred from the Trichoderma Minimal Medium Fdu plates onto PDA plates and incubated at 30° C. for 5-7 days.

To confirm that the hpt-tk selection markers had been looped out of the strains through homologous recombination of the repeats flanking these markers, isolates were screened by fungal spore PCR using a PHIRE™ Plant Direct PCR Kit. A small number of spores from each isolate was resuspended in 20 μl of Dilution buffer. The spore suspensions were used as templates in the PCRs to screen for marker loop out. The PCRs were composed of 0.5 μl of the spore suspension, 50 μmol of each primer shown below, 5 ml of 2× PHIRE™ Plant PCR Buffer, and 0.2 ml of PHIRE™ Hot Start II DNA Polymerase in a 10 μl reaction.

Forward Primer 1213333:
(SEQ ID NO: 105)
GGGACGCCCTGCTGCAACTTACC
Forward Primer 1227992:
(SEQ ID NO: 106)
GAATTCAAAAGCGCCAGTCACTGCGAG
Reverse Primer 1225982:
(SEQ ID NO: 107)
GAGACGAGACTGGAGTCGTTGCCGC

The PCRs were performed in a thermocycler programmed for 1 cycle at 98° C. for 5 minutes; 40 cycles each at 98° C. for 10 seconds and 72° C. for 1 minute and 40 seconds; and 1 cycle at 72° C. for 2 minutes. The completed PCRs were analyzed by 1% agarose gel electrophoresis using TAE buffer. Isolates with the correct diagnostic DNA band were selected for single spore purification followed by fungal spore PCR as described above.

Two isolates with the correct diagnostic DNA band for the TF 92949 gene deletion and absence of the hpt and tk selection marker genes were identified. Genomic DNA was prepared as described in Example 1 and sequenced using 2×150 bp chemistry in NEXTSEQ™ 500. Sequencing identified transformants T. reesei GMER62-DTF92949-5A1B and GMER62-DTF92949-10A1D as containing the TF 92949 gene deletion and absence of the hpt and tk selection marker genes.

### Example 19: Generation of Single Transcription Factor (TF) 70883 Deletion Trichoderma reesei Cellulase Strain

Protoplasts of Trichoderma reesei cellulase strain GMer61-A19 were generated and transformed according to Example 2 to delete the transcription factor (TF) 70883 gene. Protoplasts were transferred to 15 round-bottom polypropylene tubes and transformed with 5 μg of Bam HI-linearized and gel purified pSMai320 (Example 6). Primary transformants were selected on PDA plates containing hygromycin B. Each of the transformants was transferred to a PDA plate and incubated for 5 days at 30° C. to generate spores.

To confirm that correct integration of the linearized pSMai320 DNA had occurred through homologous recombination, primary isolates were screened by fungal spore PCR using a PHIRE™ Plant Direct PCR Kit. A small number of spores from each isolate was resuspended in 20 μl of Dilution buffer. The spore suspensions were used as templates in the PCR to screen for the integration of the linearized pSMai320. The PCRs were composed of 0.5 μl of the spore suspension, 50 μmol of each primer shown below, 5 ml of 2× PHIRE™ Plant PCR Buffer, and 0.2 ml of PHIRE™ Hot Start II DNA Polymerase in a 10 μl reaction.

Forward Primer 1225977:
(SEQ ID NO: 108)
TGACCGGGCAGGGGATCGCC
Reverse Primer 1219245:
(SEQ ID NO: 109)
CTACATCGAAGCTGAAAGCACGAGA
Reverse Primer 1225978:
(SEQ ID NO: 110)
CTGGGGCGTCAAGGGACCTGAATG

The PCRs were performed in a thermocycler programmed for 1 cycle at 98° C. for 5 minutes; 40 cycles each at 98° C. for 10 seconds, 65° C. for 10 seconds, and 72° C. for 1 minute and 30 seconds; and 1 cycle at 72° C. for 1 minute. The completed PCRs were analyzed by 1% agarose gel electrophoresis using TAE buffer. Primary isolates with the correct diagnostic DNA band were selected for single spore purification.

Single spore isolation was performed once to insure the purity of the strains prior to removal of the hpt selection marker gene. A small number of spores from each isolate was suspended in 1 ml sterile water. A 1 μl aliquot of each spore solution was mixed with 50 μl of sterile water and spread onto a 150 mm PDA plate supplemented with 1 M sucrose. The plates were incubated at 30° C. for 3-4 days.

Single colonies were transferred from the PDA-sucrose plates onto PDA plates and incubated at 30° C. for 5-7 days.

To confirm that the correct integration of the linearized pSMai320 DNA had occurred through homologous recombination, isolates were screened by fungal spore PCR using a PHIRE™ Plant Direct PCR Kit as described above. Isolates with the correct diagnostic DNA band were selected for selection marker loop out.

Spores were collected into sterile deionized water from the PDA plates that had been incubated at 30° C. for 5-7 days. The spores were counted and different amounts of spores (1×10⁷, 1×10⁶, 1×10⁵) were spread onto Trichoderma Minimal Medium plates containing 1.5 μM 5-fluoro-2′-deoxyuridine (FdU) and incubated at 30° C. for 5 days. Single colonies were transferred from the Trichoderma Minimal Medium Fdu plates onto PDA plates and incubated at 30° C. for 5-7 days.

To confirm that the hpt and tk selection marker genes had been looped out of the strains through homologous recombination of the repeats flanking these markers, isolates were screened by fungal spore PCR using a PHIRE™ Plant Direct PCR Kit. A small number of spores from each isolate was resuspended in 20 μl of Dilution buffer. The spore suspensions were used as templates in the PCRs to screen for marker loop out. The PCRs were composed of 0.5 μl of the spore suspension, 50 μmol of each primer shown below, 5 ml of 2× PHIRE™ Plant PCR Buffer, and 0.2 ml of PHIRE™ Hot Start II DNA Polymerase in a 10 μl reaction.

Forward Primer 1213333:
(SEQ ID NO: 111)
GGGACGCCCTGCTGCAACTTACC
Forward Primer 1225977:
(SEQ ID NO: 112)
TGACCGGGCAGGGGATCGCC
Reverse Primer 1225979:
(SEQ ID NO: 113)
CGCCCTTCGACGAGTCGGCAC

The PCRs were performed in a thermocycler programmed for 1 cycle at 98° C. for 5 minutes; 40 cycles each at 98° C. for 10 seconds and 72° C. for 1 minute and 40 seconds; and 1 cycle at 72° C. for 2 minutes. The completed PCRs were analyzed by 1% agarose gel electrophoresis using TAE buffer. Isolates with the correct diagnostic DNA band were selected for single spore purification followed by fungal spore PCR as described above.

Two isolates with the correct diagnostic DNA band for the TF 92949 gene deletion and absence of the hpt and tk selection marker genes were identified. Genomic DNA was prepared as described in Example 1 and sequenced using 2×150 bp chemistry in NEXTSEQ™ 500. Sequencing identified transformants T. reesei GMER62-DTF70883-8E1D and GMER62-DTF70883-11E1A as containing the TF 70883 gene deletion and absence of the hpt and tk selection marker genes

### Example 20: Preparation of Linear Plasmid DNA for Transformation and CRISPR Mediated Gene Deletion of Both Transcription Factor Genes 70883 and 92949

A total of 20 μg of either plasmid pAMFS-TF92949D (Example 16) or plasmid pSMai320 (Example 6) were combined in a 1.7 ml EPPENDORF™ tube with 10 μl of Pme I, 20 μl of CUTSMART® buffer, and brought to a final volume of 200 μl with sterile water. The reactions were incubated at 37° C. for 3 hours. The DNA was purified using MINELUTE™ columns where the reaction was divided across two columns and DNA eluted in 10 μl of sterile water per column. This yielded linear 2.977 kb and 2.688 kb DNA for pAMFS-TF92949D and linear 8.702 kb and 2.688 kb DNA for pSMai320.

### Example 21: Generation of Double Transcription Factor (TF) Deletion Cellulase Trichoderma reesei Strains

Protoplasts of Trichoderma reesei cellulase strain GMer61-A19 were generated according to Example 2. For each transformation 500 μl of protoplasts were added to a 50 ml tube and gently mixed with 1250 μl of PEG solution. Then a total of 16 μg of plasmid DNA were added to the protoplast suspension (5 μg of pSMai320 Pme I linearized DNA; 5 μg of pAMFS-TF92949D Pme I linearized DNA; 6 μg of pJFYS331 circular plasmid DNA). The transformations wee incubated at 34° C. for 30 minutes. Transformations were diluted with 15 ml of STC and mixed gently by inverting the tube several times. Each transformation was spread evenly across ten plates containing PDA supplemented 1 M sucrose. The plates were incubated at 34° C. After approximately 24 hours, the plates were covered with 20 ml of PDA+1 M sucrose containing 35 μg/ml hygromycin B. The plates were then incubated for 5 days at 30° C.

Primary isolates were transferred from the transformation plates onto PDA plates and incubated at 30° C. for 5-7 days.

To confirm that correct integration of the linearized pSMai320 and pAMFS-TF92949 DNA had integrated through CRISPR mediated gene replacement, primary isolates were screened by fungal spore PCR using a PHIRE™ Plant Direct PCR Kit. A small number of spores from each isolate was resuspended in 20 μl of Dilution buffer. The spore suspensions were used as templates in the PCRs to screen for the integration of the linearized pSMAI320 and pAMFS-TF92949D at the genes of interest. The PCRs were composed of 0.5 μl of the spore suspension, 50 μmol of each primer (3 primers for each PCR) shown below, 5 ml of 2× PHIRE™ Plant PCR Buffer, and 0.2 ml of PHIRE™ Hot Start II DNA Polymerase in a 10 μl reaction.

### PCR Screen of TF 92949 Locus:

Forward Primer 1227992:
(SEQ ID NO: 114)
GAATTCAAAAGCGCCAGTCACTGCGAG
Reverse Primer 1219245:
(SEQ ID NO: 115)
CTACATCGAAGCTGAAAGCACGAGA
Reverse Primer 1227993:
(SEQ ID NO: 116)
TCGTCGAGTCGAAGATGAGAGAGGATGG

### PCR Screen of TF 70883 Locus:

Forward Primer 1225977:
(SEQ ID NO: 117)
TGACCGGGCAGGGGATCGCC
Reverse Primer 1219245:
(SEQ ID NO: 118)
CTACATCGAAGCTGAAAGCACGAGA
Reverse Primer 1225978:
(SEQ ID NO: 119)
CTGGGGCGTCAAGGGACCTGAATG

The PCRs were performed in a thermocycler programmed for 1 cycle at 98° C. for 5 minutes; 40 cycles each at 98° C. for 10 seconds, 65° C. for 10 seconds, and 72° C. for 1 minute and 30 seconds; and 1 cycle at 72° C. for 1 minute. The PCRs were analyzed by 1% agarose gel electrophoresis using TAE buffer. Primary isolates with the correct diagnostic DNA bands for both diagnostic PCR reactions were selected for single spore purification.

Single spore isolation was performed once to insure the purity of the strains prior to removal of the hpt selection marker gene. A small number of spores from each isolate was suspended in 1 ml sterile water. A 1 μl aliquot of each spore solution was mixed with 50 μl of sterile water and spread onto a 150 mm PDA plate supplemented with 1 M sucrose. The plates were incubated at 30° C. for 3-4 days.

Single colonies were transferred from the PDA-sucrose plates onto PDA plates and incubated at 30° C. for 5-7 days. To confirm correct integration of the linearized pSMAI320 and pAMFSTF-92949 DNA through CRISPR mediated gene replacement, isolates were screened by fungal spore PCR using a PHIRE™ Plant Direct PCR Kit as described above. Isolates with the correct diagnostic DNA bands for both diagnostic PCR reactions were selected for selection marker loop out.

Spores were collected in sterile deionized water from the PDA plates that had been incubated at 30° C. for 5-7 days. The spores were counted and different amounts of spores (1×10⁷, 1×10⁶, 1×10⁵) were plated onto Trichoderma Minimal Medium plates containing 1.5 μM 5-fluoro-2′-deoxyuridine (FdU) and were incubated at 30° C. for 5 days. Single colonies were transferred from the Trichoderma Minimal Medium Fdu plates onto PDA plates and incubated at 30° C. for 5-7 days.

To confirm that the hpt and tk selection marker genes had been looped out of the TF 70883 locus through homologous recombination of the repeats flanking these markers, isolates were screened by fungal spore PCR using a PHIRE™ Plant Direct PCR Kit. A small number of spores from each isolate was resuspended in 20 μl of Dilution buffer. The spore suspensions were used as templates in the PCRs to screen for marker loop out. The PCRs were composed of 0.5 μl of the spore suspension, 50 μmol of each primer (3 primers for each PCR) shown below, 5 ml of 2× PHIRE™ Plant PCR Buffer, and 0.2 ml of PHIRE™ Hot Start II DNA Polymerase in a 10 μl reaction.

Forward Primer 1213333:
(SEQ ID NO: 120)
GGGACGCCCTGCTGCAACTTACC
Forward Primer 1225977:
(SEQ ID NO: 121
TGACCGGGCAGGGGATCGCC
Reverse Primer 1225979:
(SEQ ID NO: 122
CGCCCTTCGACGAGTCGGCAC

The PCRs were performed in a thermocycler programmed for 1 cycle at 98° C. for 5 minutes; 40 cycles each at 98° C. for 10 seconds and 72° C. for 1 minute and 40 seconds; and 1 cycle at 72° C. for 2 minutes. The PCRs were analyzed by 1% agarose gel electrophoresis using TAE buffer. Isolates with the correct diagnostic DNA band were selected for single spore purification.

Single spore isolation was performed again to insure the purity of the strains prior testing in fermentation. A small number of spores from each isolate was suspended in 1 ml sterile water. A 1 μl aliquot of each spore solution was mixed with 50 μl of sterile water and spread onto a 150 mm PDA plate supplemented with 1 M sucrose. The plates were incubated at 30° C. for 3-4 days.

Single colonies were transferred from the PDA plates supplemented with 1 M sucrose plats onto PDA plates and incubated at 30° C. for 5-7 days.

To confirm that the hpt and tk selection marker genes had been looped out of the strains through homologous recombination of the repeats flanking these markers, isolates were screened by fungal spore PCR using a PHIRE™ Plant Direct PCR Kit as described above. Two isolates with the correct diagnostic DNA band for the TF 70883 gene deletion and absence of the hpt and tk selection marker genes were identified. Genomic DNA was prepared as described in Example 1 and sequenced using 2×150 bp chemistry in NEXTSEQ™ 500. Sequencing identified transformants T. reesei GMERTFDD4D1B and T. reesei GMERTFDD7A1B as containing the TF 70883 and TF 92949 gene deletions and absence of the hpt and tk selection marker genes

### Example 22: Assessing Development of the Cellulase-Minus Phenotype of the Transcription Factor Deletion Strains in Shake-Flask Cultures

For each of the transcription factor deletion strains T. reesei GMER62-DTF92949-5A1B and T. reesei GMER62-DTF92949-10A1D (Example 18), T. reesei GMER62-DTF70883-8E1D and T. reesei GMER62-DTF70883-11E1A (Example 19), and T. reesei GMERTFDD4D1B and T. reesei GMERTFDD7A1B (Example 21), spores were suspended in a 0.8% NaCl, 0.05% TWEEN® 20 solution and counted with a hemocytometer and 2×10⁷ spores were inoculated, in triplicate, in 20 ml of Mandels-Andreotti medium supplemented with 0.1% peptone and 1% lactose in 125 ml flasks. Spores from a culture of T. reesei GMer62-1A9 (parent strain) were used as control. The cultures were incubated at 30° C. for 5 days at 160 rpm. After 5 days a 10 μl aliquot of each culture broth was mixed with 2×TCEP (0.05 M Tris(2-carboxyethyl) phosphine hydrochloride, 65.8 mM Tris-hydrochloride, 26.3% glycerol and 2.1% sodium dodecyl sulfate and heated at 98° C. for 10 minutes. After cooling down the samples were loaded onto an 8-16% TGX gel (Bio-Rad Laboratories, Inc) and ran at 150 V until the blue dye reached the bottom of the gel using a Tris-Glycine buffer system. The gel was briefly washed in water and stained with INSTANT BLUE™ (Expedeon Protein Solutions) for one hour.

The SDS-PAGE protein patterns of each culture were visually assessed by comparing the level of expression of the cellobiohydrolase I (CBHI) and cellobiohydrolase II (CBHII) proteins of the recombinant strains against the pattern of the control strain T. reesei GMer62-1A9. SDS-PAGE analysis showed that the recombinant strains had more stable protein expression patterns, than the original host strain, after being cultivated in Mandels-Andreotti medium supplemented with 0.1% peptone and 1% lactose. These strains consistently retained expression of the CBHI and CBHII.

### Example 23: Desalted BCA Assay

The protein concentration for Trichoderma reesei fermentation samples was measured after a quantitative desalting using an ECONO-PAC® 10 DG desalting column (Bio-Rad Laboratories, Inc.). A desalting column equilibrated by gravity with 50 mM sodium acetate pH 5.0 and 150 mM sodium chloride was loaded with 3 ml of filtered broth and eluted with 4 ml of equilibration buffer. The protein concentration of the Trichoderma reesei fermentation samples was then measured using a BCA Protein Assay Kit (Pierce).

### Example 24: Comparing Total Protein Titer in Trichoderma reesei Single Transcription Factor 70883 Deletion Strains, Single Transcription Factor 92949 Deletion Strains, and Double Transcription Factor Deletion Strains in 2 Liter Fermentations

Single transcription factor 70883 deletion strains T. reesei GMER62-DTF70883-8E1D and GMER62-DTF70883-11E1A (Example 19), single transcription factor 92949 deletion strains T. reesei GMER62-DTF92949-5A1B and GMER62-DTF92949-10A1D (Example 18), double transcription factor deletion strains T. reesei GMERTFDD4D1B and GMERTFDD7A1B (Example 21), and control strain T. reesei GMer62-1A9 were evaluated multiple times in 2 liter fermentations. Each strain was grown on a PDA agar plate for 4-7 days at 30° C. Three 500 ml shake flasks each containing 100 ml of Shake Flask medium were inoculated with two plugs from a PDA agar plate. The shake flasks were incubated at 28° C. for 48 hours on an orbital shaker at 250 rpm. The cultures were used as seed for fermentation.

A total of 160 ml of each seed culture was used to inoculate 3-liter glass jacketed fermentors (Applikon Biotechnology) containing 1.6 liters of Fermentation Batch medium. The fermentors were maintained at a temperature of 28° C. and pH was controlled using an Applikon 1030 control system (Applikon Biotechnology) to a set-point of 3.5+/−0.1. Air was added to the vessel at a rate of 2.5 L/minute and the broth was agitated by Rushton impeller rotating at 1100 rpm. Fermentation feed medium composed of dextrose and phosphoric acid was dosed at a rate of 0 to 10 g/L/hour for a period of 165 hours. Samples were taken at the end of the fermentation run (Day 7), centrifuged, and filtered.

Total protein titer was determined on the filtered supernatant Day 7 fermentation samples as described in Example 23. On average, a 1.11× increase in protein titer in the strains with the TF 70883 deletion was observed compared to control strain T. reesei GMer62-1A9 (Table 4). The strains with the single TF 92949 deletion had similar protein titers to control strain T. reesei GMer62-1A9. When both transcription factor genes were deleted in a single strain, on average, a 1.17× increase in protein titer was observed compared to T. reesei GMer62-1A9 (Table 4).

TABLE 4
Comparing relative protein titer in Trichoderma reesei GMer62-1A9
and transcription factor deletion strains at 7 days

| | | Relative |
|---|---|---|
| | Transcription | Protein Titer |
| Strain | Factor Gene Deletion | (Average) |
| GMer62-1A9 (host) | — | 1.00 (n = 5) |
| GMER62-DTF70883- | TF 70883 | 1.11 (n = 3) |
| 8E1D | | |
| GMER62-DTF70883- | TF 70883 | 1.10 (n = 3) |
| 11E1A | | |
| GMER62-DTF92949- | TF 92949 | 0.98 (n = 4) |
| 5A1B | | |
| GMER62-DTF92949- | TF 92949 | 0.99 (n = 4) |
| 10A1D | | |
| GMERTFDD4D1B | TF 70883 & TF | 1.16 (n = 4) |
| | 92949 | |
| GMERTFDD7A1B | TF 70883 & TF | 1.17 (n = 3) |
| | 92949 | |

### Example 25: Assessing Development of the Cellulase-Minus Phenotype of the Transcription Factor Deletion Strains in 2-Liter Fermenters

One milliliter aliquots of raw mycelia were collected from 2 L fermenters for T. reesei GMer62-1A9 (control), T. reesei GMER62-DTF70883-11E1A, T. reesei GMER62-DTF92949-5A1B, and T. reesei GMERTFDD7A1B on days 1, 3, 5 and 7 for analysis on CMC plates to determine the dynamics of the development of the cellulase-minus phenotype. Mycelial samples for day 3, 5 and 7 were diluted 1:5 in 0.8% NaCl, 0.05% TWEEN® 20 solution. The day 1 sample was not diluted. From each sample a 40 μl aliquot was spread onto a 150 mm MEX plate. The plates were then incubated at 30° C. for 5-7 days to allow for sporulation. After 5-7 days spores from the MEX plates were suspended in a 0.8% NaCl, 0.05% TWEEN® 20 solution, counted with a hemocytometer and approximately 200 spores were plated onto CMC plates. The plates were incubated at 30° C. for 2-3 days to allow individual colonies to grow.

To activate the fungal cellulases and kill the fungi, the plates were incubated at 50° C. for 3-4 hours. The colonies on each plate were counted and then the plates were stained with 10 ml of Congo Red solution for 15 minutes and washed with 1 M NaCl for 10 minutes. The colonies that were not stained and/or had a clear halo were counted to determine the percentage of colonies that were still actively producing cellulases.

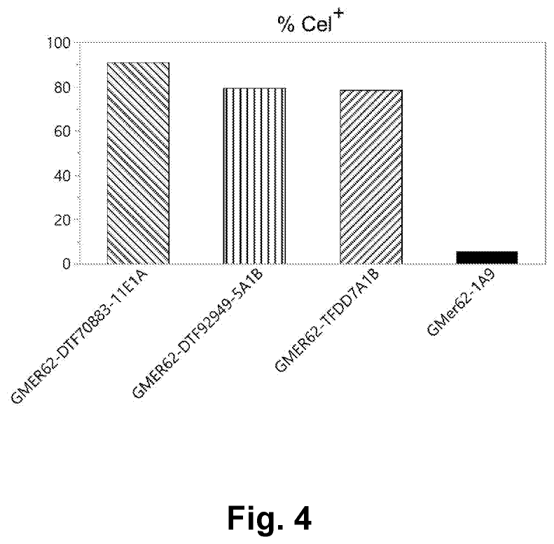

Phenotypic analysis of the T. reesei GMer62-1A9 fermentation on CMC plates showed a sharp decline in the number of cellulase producing colonies as a function of time. At day 3 only 50% of all colonies showed cellulase production on CMC plates and at day 7 only 6% of the colonies on CMC plates showed any sign of cellulase production. On the other hand, colonies from strains T. reesei GMER62-DTF70883-11E1A (deletion of transcription factor gene 70883), T. reesei GMER62-DTF92949-5A1B (deletion of transcription factor gene 92949), and T. reesei GMERTFDD7A1B (double deletion of transcription factor genes 70883 and 92949) had an average of 83% of cellulase producing colonies from day 2 through day 7 (FIG. 4).

### Example 26: Construction of Trichoderma reesei Transcription Factor 37062 Gene Deletion Plasmid pBTP01

Plasmid pBTP01 was constructed to delete the transcription factor (TF) 37062 gene in Trichoderma reesei GMER62-1A9. To construct a T. reesei TF 37062 gene deletion cassette, a PCR product (DNA fragment 1) containing a 1900 bp fragment of the upstream non-coding region of the T. reesei TF 37062 gene (SEQ ID NO: 123 for the DNA sequence and SEQ ID NO: 124 for the deduced amino acid sequence) was PCR amplified using primers 1227760 and 1227761 shown below.

Forward primer 1227760:
(SEQ ID NO: 125)
GAGTCGACCTGCAGGCATGCTTAATTAACAATTCCTCGTGACAGTTT
CTGC
Reverse primer 1227761:
(SEQ ID NO: 126)
CTTGCTCGGTCCTGGCGTAGACTTATCACAAAGTTAGCCAAACAGG

The PCR was composed of approximately 180 ng of T. reesei BTR213 genomic DNA, 20 mM dNTPs, 100 μmol of forward primer 1227760, 100 μmol of reverse primer 1227761, 1× PHUSION® HF buffer, and 2 units of PHUSION® Hot Start DNA polymerase in a final volume of 100 μl. The PCR was incubated in a thermocycler programmed for 1 cycle at 98° C. for 3 minutes; 30 cycles each at 98° C. for 10 seconds, 58° C. for 30 seconds, and 72° C. for 2 minutes; 1 cycle at 72° C. for 10 minutes; and a 10° C. hold. The resulting 1949 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 2) containing an Aspergillus nidulans amdS gene encoding the acetamidase selection marker was PCR amplified using primers 1227758 and 1227759 shown below.

Forward primer 1227758:
(SEQ ID NO: 127)
TCTACGCCAGGACCGAGCAA
Reverse primer 1227759:
(SEQ ID NO: 128)
TGGAAACGCAACCCTGAAGG

The PCR was composed of approximately 10 ng of plasmid pAILo107, a cloning plasmid containing the Aspergillus nidulans amdS gene encoding the acetamidase selection marker gene, 20 mM dNTPs, 100 μmol of forward primer 1227758, 10 μmol of reverse primer 1227759, 1× PHUSION® HF buffer, and 2 units of PHUSION® Hot Start DNA polymerase in a final volume of 100 μl. The PCR was incubated in a thermocycler programmed for 1 cycle at 98° C. for 3 minutes; 30 cycles each at 98° C. for 10 seconds, 58° C. for 30 seconds, and 72° C. for 2 minutes; 1 cycle at 72° C. for 10 minutes; and a 10° C. hold. The resulting 2718 bp PCR fragment was purified by 0.8% agarose gel electrophoresis using TAE buffer, excised from the gel, and extracted using a NUCLEOSPIN® Gel and PCR Clean-up Kit.

A PCR product (DNA fragment 3) containing an 1878 bp fragment of the downstream non-coding region of the T. reesei TF 37062 gene was PCR amplified using primers 1227762 and 1227763 shown below.

Forward primer 1227762:
(SEQ ID NO: 129)
TCCCTTCAGGGTTGCGTTTCCATGAACTACCAGCATACACGAC
Reverse primer 1227763:
(SEQ ID NO: 130)
ACAGCTATGACCATGATTACGCCTCCTTGTTTGATCCTAGCCC

The PCR was composed of approximately 180 ng of T. reesei BTR213 genomic DNA, 20 mM dNTPs, 100 μmol of forward primer 1227762, 100 μmol of reverse primer 1227763, 1× PHUSION® HF buffer, and 2 units of PHUSION® Hot Start DNA polymerase in a final volume of 100 μl. The PCR was incubated in a thermocycler programmed for 1 cycle at 98° C. for 3 minutes; 30 cycles each at 98° C. for 10 seconds, 58° C. for 30 seconds, and 72° C. for 2 minutes; 1 cycle at 72° C. for 10 minutes; and a 10° C. hold. The resulting 1923 bp PCR fragment was purified by 0.8% agarose gel electrophoresis using TAE buffer, excised from the gel, and extracted using a NUCLEOSPIN® Gel and PCR Clean-up Kit.

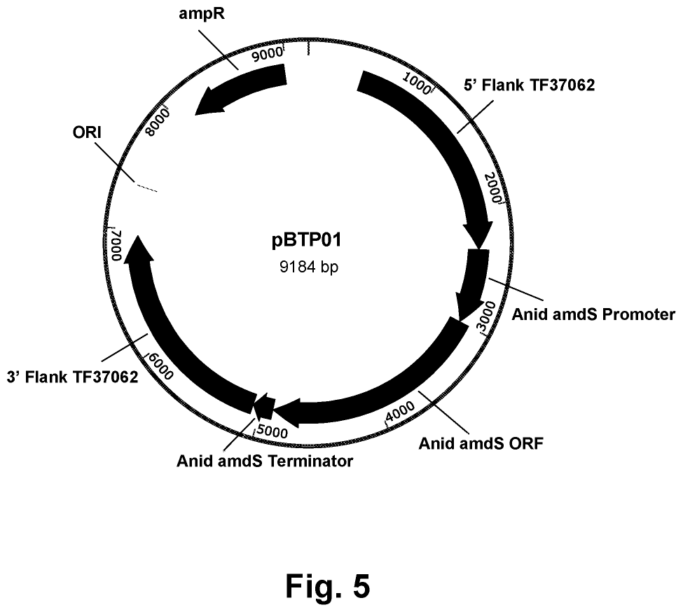

Plasmid pUC19 (New England BioLabs Inc.) was digested with Hind III and purified by 0.8% agarose gel electrophoresis using TAE buffer, where a 2,686 bp fragment was excised from the gel and extracted using a NUCLEOSPIN® Gel and PCR Clean-up Kit. The 2,686 bp fragment was assembled with the three PCR products (DNA fragments 1, 2, and 3) described above using a NEBUILDER® HiFi DNA Assembly Cloning Kit in a total volume of 20 μl composed of 1× NEBUILDER® HiFi Assembly Master Mix, and 0.05 μmol of each PCR product. The reaction was incubated at 50° C. for 60 minutes and then placed on ice. Two μl of the reaction were used to transform 50 μl of STELLAR™ chemically competent E. coli cells. The cells were heat shocked at 42° C. for 45 seconds and then 450 μl of SOC medium, pre-heated to 42° C., were added. The cells were incubated at 37° C. with shaking at 200 rpm for 60 minutes and then spread onto a 150 mm diameter 2XYT+Amp plate and incubated at 37° C. overnight. The resulting E. coli transformants were individually inoculated into 3 ml of LB+Amp medium in 14 ml round-bottom polypropylene tubes and incubated at 37° C. overnight with shaking at 200 rpm. Plasmid DNA was isolated using a BIOROBOT® 9600 and screened for proper insertion of the fragments by digestion with Nde I. A plasmid yielding the desired band sizes (5836 bp+3348 bp) was confirmed by DNA sequencing with a Model 377 XL Automated DNA Sequencer using dye-terminator chemistry (Giesecke et al., 1992, supra). One plasmid containing the insert with no PCR errors was identified and designated pBTP01 (FIG. 5).

### Example 27: Generation of Transcription Factor (TF) 37062 Deletion Trichoderma reesei Strain BTP1-BB1

Protoplasts of Trichoderma reesei cellulase strain GMer62-1A9 were generated and transformed according to Example 2 to delete the transcription factor (TF) 37062 gene. Protoplasts were transferred to 15 round-bottom polypropylene tubes and transformed with 4 μg of Pac I-linearized and gel purified pBTP01 (Example 26). Four transformants were transferred to COVE2 μlates and incubated for 5 days at 30° C. to generate spores.

The transformants of T. reesei GMer62-1A9 were screened by a fungal spore PCR method using a PHIRE™ Plant Direct PCR Kit for the homologous integration of the acetamidase (amdS) marker to the TF 37062 locus. A small amount of spores from each transformant was suspended in 20 μl of Dilution buffer (PHIRE™ Plant Direct PCR Kit). The spore suspensions were used as templates in the PCRs to screen for the TF 37062 gene deletion. Each reaction was composed of 1.5 μl of the spore suspension, 10 μmol of each primer shown below (3 primers for each PCR), 10 μl of 2× PHIRE™ Plant PCR Buffer, and 0.2 μl of PHIRE™ Hot Start II DNA Polymerase in a 20 μl reaction.

### PCR Screen of 5′ End of TF 37062 Locus:

Forward primer 1228448:
(SEQ ID NO: 131)
ATGCCCAGTCGCGAATAATCACTCAGCC
Reverse primer 1228459:
(SEQ ID NO: 132)
GGCTGAGTAGTGCTGCCATTGGGTG
Reverse primer 1228447:
(SEQ ID NO: 133)
CCATAAGGTGGCGTTGTTACATCTCCCTGAGAG

### PCR Screen of 3′ End of TF 37062 Locus:

Forward primer 1228551:
(SEQ ID NO: 134)
CCGTCCTCGGTCAGGAGCCTTGG
Forward primer 1228564:
(SEQ ID NO: 135)
CTTACTTCTTCAAATCCAGTCATGGTTGGCCTGTG
Reverse primer 1228552:
(SEQ ID NO: 136)
CCCCTATCCTCCTTGCCGTCTTGCTTTG

The PCRs were incubated in a thermocycler programmed for 1 cycle at 98° C. for 5 minutes; 40 cycles each at 98° C. for 5 seconds and 72° C. for 70 seconds; 1 cycle at 72° C. for 1 minute; and a 4° C. hold. The completed PCRs were analyzed by 1% agarose gel electrophoresis using TAE buffer. Transformants having correct targeting of the pBTP01 construct to the TF 37062 locus produced a 3 kb PCR fragment for the 5′ and 3′ end PCRs.

One transformant, T. reesei BTP1-B, that produced the correct PCR fragment was chosen for single spore isolation. A small number of spores from a 6 day old COVE2 μlate were collected in 5 ml of 0.01% TWEEN® 20 solution. A 2 μl aliquot of the spore solution was mixed with 100 μl of a 0.01% TWEEN® 20 solution and spread onto a 150 mm COVE plate. The plate was incubated at 30° C. for 5-7 days. Single colonies were transferred onto PDA plates and incubated at 30° C. for 5-7 days. Fungal spore PCR was utilized to identify spore isolates with correct targeting to the TF 37062 locus as described above. Isolates that showed correct excision of the marker genes underwent another round of single isolation followed by fungal spore PCR as described above. Genomic DNA was prepared as described in Example 1 and sequenced using 2×150 bp chemistry in NEXTSEQ™ 500. Sequencing identified transformant T. reesei BTP1-BB1 as containing the TF 37062 gene deletion.

### Example 28: Assessing Development of the Cellulase-Minus Phenotype of the Transcription Factor Deletion Strains in Shake-Flask Cultures

Spores of TF 37062 deletion strain T. reesei BTP-BB1 (Example 27) were suspended in a 0.8% NaCl, 0.05% TWEEN® 20 solution and counted with a hemocytometer and 2×10⁷ spores were inoculated, in triplicate, in 20 ml of Mandels-Andreotti medium supplemented with 0.1% peptone and 1% lactose in 125 ml flasks. Spores from a culture of T. reesei GMer62-1A9 were used as control. The cultures were incubated at 30° C. for 5 days at 160 rpm. After 5 days 10 μl aliquot of each culture broth was mixed with 2×TCEP (0.05 M Tris(2-carboxyethyl) phosphine hydrochloride, 65.8 mM Tris-hydrochloride, 26.3% glycerol and 2.1% sodium dodecyl sulfate and heated at 98° C. for 10 minutes. After cooling down the samples were loaded onto an 8-16% TGX gel and ran at 150 V until the blue dye reached the bottom of the gel using a Tris-Glycine buffer system. The gel was briefly washed in water and stained with INSTANT BLUE™ for one hour.

The SDS-PAGE protein patterns of each culture were visually assessed by comparing the level of expression of the cellobiohydrolase I (CBHI) and cellobiohydrolase II (CBHII) proteins of the recombinant strains against the pattern of the control strain T. reesei GMer62-1A9. SDS-PAGE analysis showed that T. reesei BTP-BB1 had a more stable protein expression pattern than T. reesei GMer62-1A9 after being cultivated in Mandels-Andreotti medium supplemented with 0.1% peptone and 1% lactose. T. reesei BTP-BB1 consistently retained the expression of CBHI and CBHII.

### Example 29: Comparing Total Protein Titer in Trichoderma reesei GMer62-1A9, and Transcription Factor 37062 Deletion Strain BTP1-BB1 in 2 Liter Fermentations

Transcription factor 37062 deletion strain T. reesei BTP1-BB1 (Example 27) and control strain T. reesei GMer62-1A9 were evaluated in 2 liter fermentations multiple times. Each strain was grown on a PDA agar plate for 4-7 days at 30° C. Three 500 ml shake flasks each containing 100 ml of Shake Flask medium were inoculated with two plugs from a PDA agar plate. The shake flasks were incubated at 28° C. for 48 hours on an orbital shaker at 250 rpm. The cultures were used as seed for fermentation.

A total of 160 ml of each seed culture was used to inoculate 3-liter glass jacketed fermentors (Applikon Biotechnology) containing 1.6 liters of Fermentation Batch medium. The fermentors were maintained at a temperature of 28° C. and pH was controlled using an Applikon 1030 control system (Applikon Biotechnology) to a set-point of 3.5+/−0.1. Air was added to the vessel at a rate of 2.5 L/minute and the broth was agitated by Rushton impeller rotating at 1100 rpm. Fermentation feed medium composed of dextrose and phosphoric acid was dosed at a rate of 0 to 10 g/L/hour for a period of 165 hours. Samples were taken at the end of the fermentation run (Day 7), centrifuged, and filtered.

Total protein titer was determined on the filtered supernatant Day 7 fermentation samples as described in Example 23. On average, a 1.07× increase in protein titer in the strain with the TF 37062 deletion was observed compared to control strain T. reesei GMer62-1A9 (Table 5).

TABLE 5
Comparing relative protein titer in Trichoderma reesei strain
GMer62-1A9 and transcription factor deletion strain BTP1-BB1
at 7 days

| | Transcription Factor | Relative Protein |
|---|---|---|
| Strain | Gene Deletion | Titer (Average) |
| GMer62-1A9 | — | 1.00 (n = 5) |
| BTP1-BB1 | TF37062 | 1.07 (n = 6) |

The present invention is further described by the following numbered paragraphs:

Paragraph 1. An isolated mutant of a parent filamentous fungal cell, comprising a coding sequence of a polypeptide of interest under the transcriptional control of a promoter regulated by one or more transcription factors selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;

wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii).

Paragraph 2. The mutant of paragraph 1, wherein the transcription factor comprises an amino acid sequence having at least 80% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 3. The mutant of paragraph 1, wherein the transcription factor comprises an amino acid sequence having at least 85% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 4. The mutant of paragraph 1, wherein the transcription factor comprises an amino acid sequence having at least 90% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 5. The mutant of paragraph 1, wherein the transcription factor comprises an amino acid sequence having at least 95% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 6. The mutant of paragraph 1, wherein the transcription factor comprises or consists of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 7. The mutant of paragraph 1, wherein the transcription factor is encoded by a polynucleotide comprising a nucleotide sequence having at least 80% sequence identity to 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 8. The mutant of paragraph 1, wherein the transcription factor is encoded by a polynucleotide comprising a nucleotide sequence having at least 85% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 9. The mutant of paragraph 1, wherein the transcription factor is encoded by a polynucleotide comprising a nucleotide sequence having at least 90% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 10. The mutant of paragraph 1, wherein the transcription factor is encoded by a polynucleotide comprising a nucleotide sequence having at least 95% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 11. The mutant of paragraph 1, wherein the transcription factor is encoded by a polynucleotide comprising or consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 12. The mutant of paragraph 1, wherein the transcription factor is encoded by a polynucleotide that hybridizes under very high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 13. The mutant of any one of paragraphs 1-12, wherein the promoter is a promoter from a cellulase gene.

Paragraph 14. The mutant of paragraph 13, wherein the cellulase gene is a cellobiohydrolase gene.

Paragraph 15. The mutant of paragraph 14, wherein the cellobiohydrolase gene is a cellobiohydrolase I gene.

Paragraph 16. The mutant of paragraph 15, wherein the cellobiohydrolase I gene is a Trichoderma cellobiohydrolase I gene.

Paragraph 17. The mutant of paragraph 16, wherein the cellobiohydrolase I gene is a Trichoderma reesei cellobiohydrolase I gene.

Paragraph 18. The mutant of paragraph 14, wherein the cellobiohydrolase gene is a cellobiohydrolase II gene.

Paragraph 19. The mutant of paragraph 18, wherein the cellobiohydrolase II gene is a Trichoderma cellobiohydrolase II gene.

Paragraph 20. The mutant of paragraph 19, wherein the cellobiohydrolase II gene is a Trichoderma reesei cellobiohydrolase II gene.

Paragraph 21. The mutant of any one of paragraphs 1-20, wherein the promoter is native to the coding sequence of the polypeptide of interest.

Paragraph 22. The mutant of any one of paragraphs 1-20, wherein the promoter is heterologous to the coding sequence of the polypeptide of interest.

Paragraph 23. The mutant of any one of paragraphs 1-22, wherein the polypeptide of interest is native to the parent filamentous fungal cell or the mutant thereof.

Paragraph 24. The mutant of any one of paragraphs 1-22, wherein the polypeptide of interest is heterologous to the parent filamentous fungal cell or the mutant thereof.

Paragraph 25. The mutant of any one of paragraphs 1-24, wherein the polypeptide of interest is an antibody, an antigen, an antimicrobial peptide, an enzyme, a growth factor, a hormone, an immunodilator, a neurotransmitter, a receptor, a reporter protein, a structural protein, or a transcription factor.

Paragraph 26. The mutant of any one of paragraphs 1-24, wherein the polypeptide of interest is a cellulase.

Paragraph 27. The mutant of paragraph 26, wherein the cellulase is an endoglucanase, a cellobiohydrolase, or a beta-glucosidase.

Paragraph 28. The mutant of any one of paragraphs 1-24, wherein the polypeptide of interest is a hemicellulase.

Paragraph 29. The mutant of paragraph 28, wherein the hemicellulase is a xylanase, an acetylxylan esterase, a feruloyl esterase, an arabinofuranosidase, a xylosidase, or a glucuronidase.

Paragraph 30. The mutant of any one of paragraphs 1-29, wherein the parent filamentous fungal cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

Paragraph 31. The mutant of paragraph 30, wherein the parent filamentous fungal cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Paragraph 32. The mutant of any one of paragraphs 1-29, wherein the parent filamentous fungal cell is a Trichoderma cell.

Paragraph 33. The mutant of paragraph 32, wherein the Trichoderma cell is a Trichoderma reesei cell.

Paragraph 34. The mutant of any one of paragraphs 1-33, wherein the mutant is completely deficient in the production of the transcription factor.

Paragraph 35. The mutant of any one of paragraphs 1-33, wherein the mutant is partially deficient in the production of the transcription factor.

Paragraph 36. The mutant of any one of paragraphs 1-35, wherein the modification of the one or more transcription factor genes increases the productivity of the mutant in the production of the polypeptide of interest when cultivated under the same conditions as the parent filamentous fungal cell without the modification of the one or more transcription factor genes.

Paragraph 37. The mutant of any one of paragraphs 1-35, wherein the modification of the one or more transcription factor genes reduces or eliminates the cellulase-negative phenotype in the resulting mutant compared to the parent filamentous fungal cell without the modification of the one or more transcription factor genes.

Paragraph 38. The mutant of any one of paragraphs 1-35, wherein (i) the modification of the one or more transcription factor genes increases the productivity of the mutant in the production of the polypeptide of interest when cultivated under the same conditions as the parent filamentous fungal cell without the modification of the one or more transcription factor genes, and (ii) the modification of the one or more transcription factor genes reduces or eliminates the cellulase-negative phenotype in the resulting mutant compared to the parent filamentous fungal cell without the modification of the one or more transcription factor genes.

Paragraph 39. The mutant of paragraph 36 or 38, wherein the productivity of the mutant in the production of the polypeptide of interest is increased 1%, 2%, 3%, 4%, 5% 6%, 7%, 8%, 9%, 10%, 11%, 12%, 13%, 14%, 15%, 16%, 17%, 18%, 19%, or 20% compared to the parent filamentous fungal cell.

Paragraph 40. The mutant of paragraph 36 or 38, wherein the productivity of the mutant in the production of the polypeptide of interest is increased at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% compared to the parent filamentous fungal cell.

Paragraph 41. A method of producing a polypeptide of interest, comprising cultivating the mutant filamentous fungal cell of any one of paragraphs 1-40 in a medium for production of the polypeptide of interest.

Paragraph 42. The method of paragraph 41, further comprising recovering the polypeptide of interest from the cultivation medium.

Paragraph 43. A method for constructing a mutant of a parent filamentous fungal cell, comprising modifying one or more genes each encoding a transcription factor in the parent filamentous fungal cell to produce the mutant, wherein the parent filamentous fungal cell or the mutant thereof comprises a coding sequence of a polypeptide of interest under the transcriptional control of a promoter regulated by one or more of the transcription factors, wherein the one or more transcription factors are selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;

wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii); and optionally recovering the mutant.

Paragraph 44. The method of paragraph 43, wherein the transcription factor comprises an amino acid sequence having at least 80% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 45. The method of paragraph 43, wherein the transcription factor comprises an amino acid sequence having at least 85% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 46. The method of paragraph 43, wherein the transcription factor comprises an amino acid sequence having at least 90% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 47. The method of paragraph 43, wherein the transcription factor comprises an amino acid sequence having at least 95% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 48. The method of paragraph 43, wherein the transcription factor comprises or consists of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 49. The method of paragraph 43, wherein the transcription factor is encoded by a polynucleotide comprising a nucleotide sequence having at least 80% sequence identity to 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 50. The method of paragraph 43, wherein the transcription factor is encoded by a polynucleotide comprising a nucleotide sequence having at least 85% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 51. The method of paragraph 43, wherein the transcription factor is encoded by a polynucleotide comprising a nucleotide sequence having at least 90% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 52. The method of paragraph 43, wherein the transcription factor is encoded by a polynucleotide comprising a nucleotide sequence having at least 95% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 53. The method of paragraph 43, wherein the transcription factor is encoded by a polynucleotide comprising or consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 54. The method of paragraph 43, wherein the transcription factor is encoded by a polynucleotide that hybridizes under very high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 55. The method of any one of paragraphs 43-54, wherein the promoter is a promoter from a cellulase gene.

Paragraph 56. The method of paragraph 55, wherein the cellulase gene is a cellobiohydrolase gene.

Paragraph 57. The method of paragraph 56, wherein the cellobiohydrolase gene is a cellobiohydrolase I gene.

Paragraph 58. The method of paragraph 57, wherein the cellobiohydrolase I gene is a Trichoderma cellobiohydrolase I gene.

Paragraph 59. The method of paragraph 58, wherein the cellobiohydrolase I gene is a Trichoderma reesei cellobiohydrolase I gene.

Paragraph 60. The method of paragraph 56, wherein the cellobiohydrolase gene is a cellobiohydrolase II gene.

Paragraph 61. The method of paragraph 60, wherein the cellobiohydrolase II gene is a Trichoderma cellobiohydrolase II gene.

Paragraph 62. The method of paragraph 61, wherein the cellobiohydrolase II gene is a Trichoderma reesei cellobiohydrolase II gene.

Paragraph 63. The method of any one of paragraphs 43-62, wherein the promoter is native to the coding sequence of the polypeptide of interest.

Paragraph 64. The method of any one of paragraphs 43-62, wherein the promoter is heterologous to the coding sequence of the polypeptide of interest.

Paragraph 65. The method of any one of paragraphs 43-64, wherein the polypeptide of interest is native to the parent filamentous fungal cell or the mutant thereof.

Paragraph 66. The method of any one of paragraphs 43-64, wherein the polypeptide of interest is heterologous to the parent filamentous fungal cell or the mutant thereof. Paragraph 67. The method of any one of paragraphs 43-66, wherein the polypeptide of interest is an antibody, an antigen, an antimicrobial peptide, an enzyme, a growth factor, a hormone, an immunodilator, a neurotransmitter, a receptor, a reporter protein, a structural protein, or a transcription factor.

Paragraph 68. The method of any one of paragraphs 43-66, wherein the polypeptide of interest is a cellulase.

Paragraph 69. The method of paragraph 68, wherein the cellulase is an endoglucanase, a cellobiohydrolase, or a beta-glucosidase.

Paragraph 70. The method of any one of paragraphs 43-66, wherein the polypeptide of interest is a hemicellulase.

Paragraph 71. The method of paragraph 70, wherein the hemicellulase is a xylanase, an acetylxylan esterase, a feruloyl esterase, an arabinofuranosidase, a xylosidase, or a glucuronidase.

Paragraph 72. The method of any one of paragraphs 43-71, wherein the parent filamentous fungal cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

Paragraph 73. The method of paragraph 61, wherein the parent filamentous fungal cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Paragraph 74. The method of any one of paragraphs 43-71, wherein the parent filamentous fungal cell is a Trichoderma cell.

Paragraph 75. The method of paragraph 74, wherein the Trichoderma cell is a Trichoderma reesei cell.

Paragraph 76. The method of any one of paragraphs 43-75, wherein the mutant filamentous fungal cell is completely deficient in the production of the transcription factor.

Paragraph 77. The method of any one of paragraphs 43-75, wherein the mutant filamentous fungal cell is partially deficient in the production of the transcription factor.

Paragraph 78. The method of any one of paragraphs 43-77, wherein the modification of the one or more transcription factor genes increases the productivity of the mutant in the production of the polypeptide of interest when cultivated under the same conditions as the parent filamentous fungal cell without the modification of the one or more transcription factor genes.

Paragraph 79. The method of any one of paragraphs 43-77, wherein the modification of the one or more transcription factor genes reduces or eliminates the cellulase-negative phenotype in the resulting mutant compared to the parent filamentous fungal cell without the modification of the one or more transcription factor genes.

Paragraph 80. The method of any one of paragraphs 43-77, wherein (i) the modification of the one or more transcription factor genes increases the productivity of the mutant in the production of the polypeptide of interest when cultivated under the same conditions as the parent filamentous fungal cell without the modification of the one or more transcription factor genes, and (ii) the modification of the one or more transcription factor genes reduces or eliminates the cellulase-negative phenotype in the resulting mutant compared to the parent filamentous fungal cell without the modification of the one or more transcription factor genes.

Paragraph 81. The method of paragraph 78 or 80, wherein the productivity of the mutant in the production of the polypeptide of interest is increased 1%, 2%, 3%, 4%, 5% 6%, 7%, 8%, 9%, 10%, 11%, 12%, 13%, 14%, 15%, 16%, 17%, 18%, 19%, or 20% compared to the parent filamentous fungal cell.

Paragraph 82. The method of paragraph 78 or 80, wherein the productivity of the mutant in the production of the polypeptide of interest is increased at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% compared to the parent filamentous fungal cell.

Paragraph 83. An isolated transcription factor, selected from the group consisting of:

(a) a transcription factor comprising an amino acid sequence having at least 70% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;

(b) a transcription factor encoded by a polynucleotide comprising a nucleotide sequence having at least 70% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and

(c) a transcription factor encoded by a polynucleotide that hybridizes under high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 84. The transcription factor of paragraph 83, having at least at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124.

Paragraph 85. The transcription factor of paragraph 83, encoded by a polynucleotide comprising a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 86. The transcription factor of paragraph 83, encoded by a polynucleotide that hybridizes under very high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

Paragraph 87. An isolated polynucleotide encoding the transcription factor of any one of paragraphs 83-86.

Paragraph 88. A nucleic acid construct or expression vector comprising the polynucleotide of paragraph 87 operably linked to one or more control sequences that direct the production of the transcription factor in an expression host.

Paragraph 89. A recombinant host cell comprising the polynucleotide of paragraph 87 operably linked to one or more control sequences that direct the production of the transcription factor.

Paragraph 90. A method of producing a transcription factor, comprising cultivating the recombinant host cell of paragraph 89 under conditions conducive for production of the transcription factor.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An isolated mutant of a parent filamentous fungal cell, comprising a coding sequence of a polypeptide of interest under the transcriptional control of a promoter regulated by one or more transcription factors selected from the group consisting of:
(a) a transcription factor comprising an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;
(b) a transcription factor encoded by a polynucleotide comprising a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and
(c) a transcription factor encoded by a polynucleotide that hybridizes under high or very stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;
wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii).

2. The mutant of claim 1, wherein the promoter is a promoter from a cellulase gene.

3. The mutant of claim 2, wherein the cellulase gene is a cellobiohydrolase gene.

4. The mutant of claim 1, wherein the promoter is native or heterologous to the coding sequence of the polypeptide of interest.

5. The mutant of claim 1, wherein the polypeptide of interest is native or heterologous to the parent filamentous fungal cell or the mutant thereof.

6. The mutant of claim 5, wherein the polypeptide of interest is an antibody, an antigen, an antimicrobial peptide, an enzyme, a growth factor, a hormone, an immunodilator, a neurotransmitter, a receptor, a reporter protein, a structural protein, or a transcription factor.

7. The mutant of claim 7, wherein the enzyme is a cellulase or a hemicellulase.

8. The mutant of claim 1, wherein the parent filamentous fungal cell is a Trichoderma reesei cell.

9. The mutant of claim 1, wherein the mutant is partially or completely deficient in the production of the transcription factor.

10. The mutant of claim 1, wherein the productivity of the mutant in the production of the polypeptide of interest is increased at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% compared to the parent filamentous fungal cell.

11. A method of producing a polypeptide of interest, comprising cultivating the mutant filamentous fungal cell of claim 1 in a medium for production of the polypeptide of interest, and optionally recovering the polypeptide of interest from the cultivation medium.

12. A method for constructing a mutant of a parent filamentous fungal cell, comprising modifying one or more genes each encoding a transcription factor in the parent filamentous fungal cell to produce the mutant, wherein the parent filamentous fungal cell or the mutant thereof comprises a coding sequence of a polypeptide of interest under the transcriptional control of a promoter regulated by one or more of the transcription factors, wherein the one or more transcription factors are selected from the group consisting of:
(a) a transcription factor comprising an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;
(b) a transcription factor encoded by a polynucleotide comprising a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and
(c) a transcription factor encoded by a polynucleotide that hybridizes under high or very stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123;
wherein the one or more transcription factor genes are modified in the parent filamentous fungal cell to produce the mutant rendering the mutant partially or completely deficient in the production of the one or more transcription factors, wherein (i) the modification of the one or more transcription factor genes increases the productivity of the mutant in the production of the polypeptide of interest when cultivated under the same conditions as the parent filamentous fungal cell without the modification of the one or more transcription factor genes, (ii) the modification of the one or more transcription factor genes reduces or eliminates the cellulase-negative phenotype in the resulting mutant compared to the parent filamentous fungal cell without the modification of the one or more transcription factor genes, or (iii) the modification of the one or more transcription factor genes results in a combination of (i) and (ii); and optionally recovering the mutant.

13. The method of claim 12, wherein the promoter is a promoter from a cellulase gene.

14. The method of claim 12, wherein the promoter is native or heterologous to the coding sequence of the polypeptide of interest

15. The method of claim 12, wherein the polypeptide of interest is native or heterologous to the parent filamentous fungal cell or the mutant thereof

16. The method of claim 12, wherein the mutant is partially or completely deficient in the production of the transcription factor.

17. The method of claim 12, wherein the productivity of the mutant in the production of the polypeptide of interest is increased at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% compared to the parent filamentous fungal cell.

18. An isolated transcription factor, selected from the group consisting of:
(a) a transcription factor comprising an amino acid sequence having at least at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, or 124;
(b) a transcription factor encoded by a polynucleotide comprising a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123; and
(c) a transcription factor encoded by a polynucleotide that hybridizes under high or very high stringency conditions with the full-length complement of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, or 123.

19. A recombinant host cell comprising a polynucleotide encoding the transcription factor of claim 18, wherein the polynucleotide is operably linked to one or more control sequences that direct the production of the transcription factor.

20. A method of producing a transcription factor, comprising cultivating the recombinant host cell of claim 19 under conditions conducive for production of the transcription factor.


SEQUENCE LISTING (136 sequences)

SEQ ID NO: 1 - DNA, 2315 bp, Trichoderma reesei
atgcttcgaccggcgacgcccttgaccgtgctgcttggaattgcctttgctttgctgctcctatccgtcttgtcggcccccatcatctcggccatccccctcggcagttctcaggatgtccagtttggcgtctttggcttttgccggccctcgggctgcagcagcgtcggcattggctatgacattggtgagaatacccatcattacatgaccccgtgcttgtgcccatgattctccgtgtcctcctcagcctcgactccatatttgattgctgggcttgcaatgtttcccaacaaatggcagcgcggtaactaacaagaatgtgcgtatagcgagcgtcctcaccgacacgaagagctcgtcgtttgatctgcccagtggcgttcgccatacgctctcttcaatcctgatacttcaccccgtcgccgcctttctcaccctcgtcatgcttctgatggccgttgctgcccacttccgcgctgcttcgcactcgtcaaggtatctgctgttcttcctcgtcttcaacctcgtcacgttgctcgtgtgtgtggctgctttcgtcatcgacgtcctactcttcatacctcacatggcatggggcacctacattgttttggctgccactgttctcgttttcttgagtcttctggtctcttgcgccatgcgacgcactctggttagcaggaaagaccgaaagcggagaattgcagagaatgccgagatgagcggagagaactactacaaccgagagaatcaggcgccgaaatctacctttaccatgacgacccagcctaccatgccagttgtgagtggtggcaacggcacacaagacatcctgcctacgttcgctacgtacgagactccaagagatgaccaagtcagcgacgagcgcatccctctaacacagcgaaccacatccgaaagatcgccccatggaaggcagcccgcgcccgccgacatggctgccgttgcgggcagcgagattggcagtgcttacagcggaccgcagaggtccgcatcccaggactcgtatggcaactacggcaacgtgccacccaacgcctacggacaagccggccagccgtacgatggtcgctcgaatcgagtccccatcaggagagagacggaccccgtcaatggctatggcgctggcggccgtggcggctatggagcccccatgagaggacggggcggctatggccctcccggccggggtggatacggcccgcgaggcggtggacgtggaggatacggaccgccatctcgcgcaggctacaatggctcatactaccacatgcccaggggcggaggacgtggcccttcgccgatgaacaaccctaatgatggcgtggccgggcaatacaacagaatgccgtcgccatcgcagcaaggctatgctggagcaccgcagcagccgctacaggcagacgtccccctaaacccggctccgtccggcgccggcaacacatacgtcccttaccgcccgaatatcgatctgccaagggctgagtcccccccaccactgccggaacctaccgaatctgccgtctcgtcggtaccaaacgccattgagatggatgcgacgccggctgccgcggcaagcagcaacaatggccagttcggcaacctcagagatagcgacacagacgttgccggaatggtggacctccaacagggccagcagcccttatccacgcagagagacacgttcgccagcgatgggagcaggtactctcaagatgagtatgtattgtactgtgttgcagcctccctccacccaggtaccctcgccgttcatcaagtaggtggctaaccaattcgaaagtacatctaggcaatttgccccctctcgtgcccagtggaaccaagaggctgggagagactcacctcgggtacccgtagcaaactcgtcgcgcggacctaccccgaatatgtctggcatgactgcgccagtggttgatgccaagggcaagggcgactactacgaagacgtcgaccctcgttttgaccaaacagtttatacacccccgcgtcgccagactcctccgccgccgctgcagctaaacacggtcgactacgaagatatacgagttcccgccagcggcactcgcagccccgccgaatcggaacactccaactttacctccatctcgcaacgtgggatcaacccacaatggagcccgcaacccccagtgccccaacgccgaccagtacaacagcgccatgacatgatcctggacaatcccgatttcaagctcccgggaggtcgagcgacggcagcggggaagcgcagaggaccagggatgatgccgtag

SEQ ID NO: 2 - protein, 692 aa, Trichoderma reesei
MLRPATPLTVLLGIAFALLLLSVLSAPIISAIPLGSSQDVQFGVFGFCRPSGCSSVGIGYDIASVLTDTKSSSFDLPSGVRHTLSSILILHPVAAFLTLVMLLMAVAAHFRAASHSSRYLLFFLVFNLVTLLVCVAAFVIDVLLFIPHMAWGTYIVLAATVLVFLSLLVSCAMRRTLVSRKDRKRRIAENAEMSGENYYNRENQAPKSTFTMTTQPTMPVVSGGNGTQDILPTFATYETPRDDQVSDERIPLTQRTTSERSPHGRQPAPADMAAVAGSEIGSAYSGPQRSASQDSYGNYGNVPPNAYGQAGQPYDGRSNRVPIRRETDPVNGYGAGGRGGYGAPMRGRGGYGPPGRGGYGPRGGGRGGYGPPSRAGYNGSYYHMPRGGGRGPSPMNNPNDGVAGQYNRMPSPSQQGYAGAPQQPLQADVPLNPAPSGAGNTYVPYRPNIDLPRAESPPPLPEPTESAVSSVPNAIEMDATPAAAASSNNGQFGNLRDSDTDVAGMVDLQQGQQPLSTQRDTFASDGSRYSQDEQFAPSRAQWNQEAGRDSPRVPVANSSRGPTPNMSGMTAPVVDAKGKGDYYEDVDPRFDQTVYTPPRRQTPPPPLQLNTVDYEDIRVPASGTRSPAESEHSNFTSISQRGINPQWSPQPPVPQRRPVQQRHDMILDNPDFKLPGGRATAAGKRRGPGMMP

SEQ ID NO: 3 - DNA, 1224 bp, Trichoderma reesei
atggtatcgccgtacgaagattcgctccgacgaaactcgtcattcaggtcccttgacgccccagctaccgatgccggctcgcgctcgtaccaggagagagccacggcgcctgagccagtgcactacaattcttcctacccgcgcagtccgcatgccatggaacccctggcgtacttgagcatggactttagcattgtcaaagcatcgcccatgttcatggaaatcgtccacgcatcgaacccactggggaacaaattgagcgacatcgtcacgccacatcaggcgagtttccttaacaacctccagaaccagctcatcgaagagcaacgactgcacgagcccaattatctgcctcccatgctgggccgactggatcttgccatcaaagagtttgggtttacgagtcaagatgctgcgaaatttcgcctcaaccatctcgaatattttggctttgttggatacgacggttacacgaggacctttccgcttcgttttgggcttgcaaaggagggttcgttttatttcgttgtgttcttgttgagtcttcagcatcacccgccgctaccgccgcccttgccatcacagcaacatgcgtcccagacacaacagcaacggactcagcaacatcaacatcaacagcattcacagtctcgggagccccatcagcagacacaacaacaacagcagcagcagcaacaacaacaacaacaacaacagactcagcagcgccagcaatatccgcagcatttgcatcagagacaactttcgcagccagctccatacacgcagctgccgcagcactcaccggttacgcaccatacacgggaacctactgccgcctcctatcatcttccgccgcccccgcatcaaccggcatacggagatcggtcgagcaatccctcagcagatccttttcgtcatcgcctcagcgaaggatctatccggccgccacacaatccggctgcctcgagtgccggtacaagctcaagcctccagtcttattcacagtcacactactcccggccaaattaccaggcgtcgcagaacgaacatgtctctactagccggcctgcaactcagccctcctatcagctaccccctattcgggcgccgcctgaacaccggcctgcgtcccgagaggtcggttggcccaatggcgagcgatcaagacgagtcgatatcggtggcttgatagaacagcctggggattccaccaggagatga

SEQ ID NO: 4 - protein, 407 aa, Trichoderma reesei
MVSPYEDSLRRNSSFRSLDAPATDAGSRSYQERATAPEPVHYNSSYPRSPHAMEPLAYLSMDFSIVKASPMFMEIVHASNPLGNKLSDIVTPHQASFLNNLQNQLIEEQRLHEPNYLPPMLGRLDLAIKEFGFTSQDAAKFRLNHLEYFGFVGYDGYTRTFPLRFGLAKEGSFYFVVFLLSLQHHPPLPPPLPSQQHASQTQQQRTQQHQHQQHSQSREPHQQTQQQQQQQQQQQQQQQTQQRQQYPQHLHQRQLSQPAPYTQLPQHSPVTHHTREPTAASYHLPPPPHQPAYGDRSSNPSADPFRHRLSEGSIRPPHNPAASSAGTSSSLQSYSQSHYSRPNYQASQNEHVSTSRPATQPSYQLPPIRAPPEHRPASREVGWPNGERSRRVDIGGLIEQPGDSTRR

SEQ ID NO: 5 - DNA, 2005 bp, Trichoderma reesei
atgctgcgctactcccccgtcttacacctggatactctctccttgccaccactgaccaatgctcttccccgcccaaagtgcgagtacctcagcgctgtcgatagctgcacgcactgccgcgatgcccacgtgcagtgcactttcgacctgcccctggcgcgacgcggccccaaagcgaggaagaagagcgaccagcccggccagccgcctcctgatccgagctcgctctccaccgcggctcgacccggccagatgccgccgccgctgaccttctccggccccgcagtagccgcgctgcagcccttcgcctcgtcgtcgctgtcgcccgacgcggcctgggagcccgtcgagccgctcagcattgacaacggcctgccccggcagccgctgggcgacctgcccggcctctccaccatccagaacatctcgacgcgccagcgatggatacacctggccaacgccatgacgctgcgcaacacgacgctagagcgcgtctcgaagcgatgtatcgacctcttcttcgactacctctaccccctcacccccctggtgtacgagccggccctccgggacgtgctcgcatacatcttctcccagcccttgcctggcgtcaaccaaccatcgccgctgtcacagctcacgccagacccgaccaccggcaccacccccctcaacgctgccgagtcgtgggccggctttggccagcccagcggctcgcgaaccgtcggcagcaggctggctccctgggccgactcgaccttcaccctggtcacggccgtctgcgcagaggcagcattcatgctacccaaggacattttccccgaaggagaatccgtctctgagatcttgctcgaagcctctcgggactgcctgcaccagcacctcgaggccgacctggagaatccgacggccaactcgattgccattcgctacttccactccaactgcctccacgctgcggggaagcccaagtactcgtggcacatatttggcgaggccatccgcctggcgcaggtcatgcagctgcacgaggaggctgccctcgaggggctcgtccccatcgaggcagagttccgccgtcgctgcttttggatcctgtacttgggcgacaagtcagccgctatactcaacaatcggcccatcaccatccacaagtactgcttcgacgccggcatcaccacgctatacccgtcgggtatcgaggacgagttcctgagcacggcgtccgagccgccccggaagagcttcatatccggcttcaacgcaaatgtgcggctctggcagtccgcggctgatttgctgctggaaatccgcgtgctgcaagatcagatgatgcagcactttcgagggaccatgcccccgaaccatgtgctgccctccgccgacaggcagcatctcgattctctctatgtccgcttcatcacctgcttggacgatctcccgccgtacctccagtcgtgcactctggcgatggcagcgatggcagaaggcaacgggtctgccgagtccaagcagtacgtgatacagtgcatcaacctgcaggtgacgtttcactgtctgcgcatggtaattacgcagaaattcgaagacctctcttattttgctcctggcgttgagcaggctgatctcagaaagtcggagattgtgcgagacatgctgagggtgatgaacgaggcgcccttttggggcctgcaggccaatggcgagccaaacgtgagtcgtttccttgtctcttctcttttctgcacacccttttcttcgacgaccccccctctctctttatatccctgcggatatgtatatcatcaagcctcggcacttgttgctaatctgtcctgattatgttgtctggatgctgcaggttgaaaagattcgccttatcggagctagtttgctggccatcatccatcgcaaccaggattcacccttggctacgcgagccaggagcgacttttccgtgcttttggatattctcacgcggctggactcgaaggcgtcggactaa

SEQ ID NO: 6 - protein, 618 aa, Trichoderma reesei
MLRYSPVLHLDTLSLPPLTNALPRPKCEYLSAVDSCTHCRDAHVQCTFDLPLARRGPKARKKSDQPGQPPPDPSSLSTAARPGQMPPPLTFSGPAVAALQPFASSSLSPDAAWEPVEPLSIDNGLPRQPLGDLPGLSTIQNISTRQRWIHLANAMTLRNTTLERVSKRCIDLFFDYLYPLTPLVYEPALRDVLAYIFSQPLPGVNQPSPLSQLTPDPTTGTTPLNAAESWAGFGQPSGSRTVGSRLAPWADSTFTLVTAVCAEAAFMLPKDIFPEGESVSEILLEASRDCLHQHLEADLENPTANSIAIRYFHSNCLHAAGKPKYSWHIFGEAIRLAQVMQLHEEAALEGLVPIEAEFRRRCFWILYLGDKSAAILNNRPITIHKYCFDAGITTLYPSGIEDEFLSTASEPPRKSFISGFNANVRLWQSAADLLLEIRVLQDQMMQHFRGTMPPNHVLPSADRQHLDSLYVRFITCLDDLPPYLQSCTLAMAAMAEGNGSAESKQYVIQCINLQVTFHCLRMVITQKFEDLSYFAPGVEQADLRKSEIVRDMLRVMNEAPFWGLQANGEPNVEKIRLIGASLLAIIHRNQDSPLATRARSDFSVLLDILTRLDSKASD

SEQ ID NO: 7 - DNA, 3789 bp, Trichoderma reesei
atggagtgcgtcatggaggacagctcggacgataccgccaaggaaccatcagctctgaccgatggcaatttcacagaaaaagacggcgaagcagcggctgctttgggtaccagtctcacggcgattcaatcctggcagacctccgtcttgaccatcaatgataacaaccgttcagcaacacacccgtctgaccatgagtcgttgccatgtatagacacttttgaagagactccgagctctgacgaaaccgttattgagctcgtcacatccgacctgataccgcctccatctcgtctcgtcacatcatcacgcagttctacgctcaaagtctccgagcttgatgatgctgtgtcagacagtctttcgacagagacttttcgagcaacttcaccggactgggtaccttcaaccgggtccttgagcgttgagatacccggaagcgttcagcttgtaccaaggtcatgctacgagggctttcagcctcctgacgacctaccgatcttaccagagtggaaggctgttcagactctttctccagccttggcggaaagcacgcaagattttgacgagatcatgctggacgattttgcgatataccttgataaggagagatgctcacaggaaatgcgatctctacaccagctcaataccaagattggacacaacgacttctactttgacggtgttctccgcattggcgataccaggacgtatgtgcgccgcattcccatcaccgcggttcccattggcaactacggctcaatctcttatcacactgtccgagacaacatctggctgcaatctcctctgagctacaaacgggagctatattacaggttggggaagccggccaaggaatatgcacgcttcttttaccctttcttatgggtggcggacctggcgaagcactttgtggactttctctccatcatggcggagaacgagcgtagtgtttccatacaccactttcgatctacttttgcagcatggatgaagaagagccacaaggactcgcccgagtttttcagctggctacgccaacacccgagcgatgactaccggacttcgatagccgccaacatagggtttctccataaagagactctgggggtgttgaaccatcgagaggcgtattcccatgctatctgggcggagatctgggaattccaatcctttacatcaatcacgagccagcccaagactgagactccgccgccgacgattgttacgcaatacatatttgactgctttgaacatcttccttttggcgatcgcctcaaggttgtccctctgagttcacggaccacggagctccgcaacagcttgatccgacagcggcacctagagcttccgtcaaagctgcaccagacatctaaggacatctctacggcgccacaagagcggatcaagaatatccggccaggcgacacaatctcaacccatcgcgacgacgcagagtccggcacattgtggaaacgagaattgtccaaaggcttctctgacgtggacagatggtttgcgctggtgcagtcggttcacgttgacagaaaaggggtgcgtacctttgatgtgatatggtattacagacccgtggacacgctctgcgggttgatgaagtatccgtggaacaacgagctgtttctctcagaccattgctcttgctccgagaaagccaagatcgaagaaagcgaggttctcggtgtccatggcgttgacttttggggcacctcagcaacaagcgccgagctcttttgccggcagacctaccttcaagaagagaggagatgggtgacactggatgggaagcacctgcaatgtaggcacacttcaccaataccagatgatgaaatgccactcgagtatcagccagggaagacacatctgcttcgtttgaacttgaaaagtccattttccgagccttgtgagtttatgcacgcgtctgatgaaggaagccgaaggttgtattacttcaggcgactattgcggcgccgccaggttgacccaaacgctcgcgaggcgcggccaaatgaacttgtatacagcgagcagacctgcaaagtcagcagggatcgaatcatgggcccatgttcagtgagatgcttccgagatggagagatgatccctacgccgtatgaccacgacggcacgggcaacctcttttacatcactcaccgacaggtagccgccaatggacattcggtatatgtgccgcttgacgaagcaccgtcctctctgcagcaaggctttgatcctctccagaagttgcagaagcttcgcgggatggatctcttctgtggaggcggtaactttggcagagggcttgaagacggcggaggaatcgagatgaaatgggcaaatgactacgacagcaaggctatccacacgtacatggccaacgtcaaaaaccctcaagacgtccacccgtttctgggctccattgacgacttacagcgccttgccattcaaggcgacttcgcggacaacgtacccctaattggagacgttgatttcgtgtccggtggcagcccatgtcctggattctcacgcctcaccaacgacaagacgacggcagcgcaaaggaagaaccaatcgcttgttgcggcgtttgcgtcctttgttgatctctatcggcccaaatacggcgtgttggagaatgtacctgggatggtccacaagaagcacgaccgagaccgagacgtcttcagccagctcatctgcgccctagtcggcttgggctatcagacgcaattcttctacctggacgcatcgtcatgcgggtcgccgcagcgcagatcacgcatcttcatcgtctttgctgccccgggactcgagctgcccgacaagccggtgcagactcactcacacccccctaacacaagggagcatgggatcgggtggctgcccaacggccagcacatggcaagccgagaaatgccggcagcaacgcccttcaagtttgtctctgcggaagaagcgaccgccgacctgccgcccatctacgatgccaagcccgatatatgtgttcctttccccgatcaccgctcaaccttgggtatgtcagacttgctgcgagctcggatatcggtcataccgacacgaccatggggcatgaacttttcgcaagcgtggttcggggagaggaaggtcggcggctccagcacgatgacggcagccgagcgcgagttcttcatcgggacgaaaggcgagggcaagcagcccatgtcggtgcagccctactcaaattcgtacggaaggatgttccccaacaggctctttgagactgtggtgacgaggcagacgctgggcgacgcgaagaacgggcgactgctccactggcgagagaataggcccgtgaccatcatggaggcgcgcagggcgcagggcttcctcgacgaagacgtgctgctcggggaccccccgacgcagtacaggattgtaggcaacagcgtcgcgaggcagcctgctgtggctctcggtgtgacgtttcgcgaagcgtgggtggcgagcctgaagaggaacagagagctgcagcgcgcaaggagctgcgttgccgaggaacatggcggattcacgaacggcgttccagtgactcatgagtccctcgagaagatggcggacggcatgggcagcatatcgagagatcagcagctaagggcctttacgcccgcaacggacagcacactgcagcagtcgatggatatcacgatgggcagtaaacgccctcgttcggccaacatggtgattgagctgctcgagtcctcgaaacgacagaggccgaaccccttttga

SEQ ID NO: 8 - protein, 1262 aa, Trichoderma reesei
MECVMEDSSDDTAKEPSALTDGNFTEKDGEAAAALGTSLTAIQSWQTSVLTINDNNRSATHPSDHESLPCIDTFEETPSSDETVIELVTSDLIPPPSRLVTSSRSSTLKVSELDDAVSDSLSTETFRATSPDWVPSTGSLSVEIPGSVQLVPRSCYEGFQPPDDLPILPEWKAVQTLSPALAESTQDFDEIMLDDFAIYLDKERCSQEMRSLHQLNTKIGHNDFYFDGVLRIGDTRTYVRRIPITAVPIGNYGSISYHTVRDNIWLQSPLSYKRELYYRLGKPAKEYARFFYPFLWVADLAKHFVDFLSIMAENERSVSIHHFRSTFAAWMKKSHKDSPEFFSWLRQHPSDDYRTSIAANIGFLHKETLGVLNHREAYSHAIWAEIWEFQSFTSITSQPKTETPPPTIVTQYIFDCFEHLPFGDRLKVVPLSSRTTELRNSLIRQRHLELPSKLHQTSKDISTAPQERIKNIRPGDTISTHRDDAESGTLWKRELSKGFSDVDRWFALVQSVHVDRKGVRTFDVIWYYRPVDTLCGLMKYPWNNELFLSDHCSCSEKAKIEESEVLGVHGVDFWGTSATSAELFCRQTYLQEERRWVTLDGKHLQCRHTSPIPDDEMPLEYQPGKTHLLRLNLKSPFSEPCEFMHASDEGSRRLYYFRRLLRRRQVDPNAREARPNELVYSEQTCKVSRDRIMGPCSVRCFRDGEMIPTPYDHDGTGNLFYITHRQVAANGHSVYVPLDEAPSSLQQGFDPLQKLQKLRGMDLFCGGGNFGRGLEDGGGIEMKWANDYDSKAIHTYMANVKNPQDVHPFLGSIDDLQRLAIQGDFADNVPLIGDVDFVSGGSPCPGFSRLTNDKTTAAQRKNQSLVAAFASFVDLYRPKYGVLENVPGMVHKKHDRDRDVFSQLICALVGLGYQTQFFYLDASSCGSPQRRSRIFIVFAAPGLELPDKPVQTHSHPPNTREHGIGWLPNGQHMASREMPAATPFKFVSAEEATADLPPIYDAKPDICVPFPDHRSTLGMSDLLRARISVIPTRPWGMNFSQAWFGERKVGGSSTMTAAEREFFIGTKGEGKQPMSVQPYSNSYGRMFPNRLFETVVTRQTLGDAKNGRLLHWRENRPVTIMEARRAQGFLDEDVLLGDPPTQYRIVGNSVARQPAVALGVTFREAWVASLKRNRELQRARSCVAEEHGGFTNGVPVTHESLEKMADGMGSISRDQQLRAFTPATDSTLQQSMDITMGSKRPRSANMVIELLESSKRQRPNPF

SEQ ID NO: 9 - DNA, 2316 bp, Trichoderma reesei
atggcccagccgccaagtttctacgcctcttccaggcccttggaagtcttcaccgacgacatcttcttcgagaacgaagcccccatgacaagccacgcacccatgccgaatccgactaggcctattcgtcgtcctctcagcaactcaagctcgaatgtcatcctcgagcccgcagtctcccagaacagcaacaagatctcaccatacaagcccaagaccgcagcacccagagcaccgctcaagccgtcgcatggcctgaacaagttcaacggcatctcaatggctcctccctcagacaaggcgccagtcaccgactctctgcagaagaagccgcagctatccaagttcaagaccggcctgcagaagccagctctcgccagccctcacgacgccttctttggaaaggaaaacgttcagcctcaaatcttcccggcaccgccggcaatcaacatctccatggagcagtattaccaggattccaatggaaagcgaggactgatggaggcggcaccgatcaaggactctcgagtcagcagcaaaaagcccaaggccgaggagcaggtgctcccgccgcacgactcgttccctcccattactgacgatggcaccaaaccgcctcacagctatgcacagcttattggaatggccattcttcgatcttcaaaacgacgcctcacccttgcccagatttacaagtggatcagcgacaactattctttctacaaccccaatgacgcaggctggcaaaatagcattcgacacaacctgagtctgcacaagaactttatcaaaatcgagcgaccaaaggacgatcccggcaagggcaactactggggcatcgagcccggcaccgagttccagttcctcaaggaaaagcccacgcgcaaagccgtcccgactgccgagaacctccccgtcatgtccacccgcctcgagccctctcgacctacgccagcattgatgcccgagccgtgtctgccgcctcctgctcccatccatcatcatcagcaccaccaccaccagcagcatcagccgcatgcgaccagcctccctccgctgccgacttctcaggcgaccttgtccatgcctctggagccctcatcagacgccaccattcttctgtccgacaacgcgcccgccgaggacctggcggacaagggaccggacaacgaactgccattggaatcgtccttcttctcgccccttcctcccgccctcaactccactccgccggttccccggccagagcgacgaaacggcacaccgccccctgtgagccgcaatcccgcctcgtccgcaaccaggactcacaagcgcaagtttgcctccatggacgacagcggctacatctcgtctctcgagtcttccgccatgcgcccgaacgcagccaggtcggccctcttcacatccgaggcggaccgacaccgcatcaagcgcggccgggctgaggaggaaattgcacggctgagaggatcttctccctttagcccaaccaagagtcgctcgctctccagctatgggcttacttcgtcatctcctttccgtcagtccctcgacaaccagatgcctccccccctcactcccgttgtcaagaaggtcaaacccattgcgcagccaccaccttcggtgtctcccaacacgaacctgcgcatccatcgcgccaaggtccgccacatgctgcagtctcctcttcggcgtgtcgccaacctgtcgaatgaggacatgttgccgtggagccccgccttccggctggatgacagcatcttcacctttgatacgcccaatgccagcagcaacctccccgactttgacattttccaagacctgcccgtggatgatgaaaacgtctttgacagcattggcgctgcagatgcgggctcgcccattaagcgctcggccaagcgcgctcgcttggatcggtccatttcgtcgtcagccattggggagatgtcggcttcaaagaggcttctcacatcggcccctctgctcaaggcccctgacacgccttcgcagtttctggagacaccgagcaaagtctttgaaggcctcagctctccctccaagatcctccaggggtcgccaatcaggggcaactcgccaagcaagttcgtctcgctgatagagcttccaggagatgccgactggccgtcccttcacctggacaccggcgactttgcttcgggtgatacgtccgacttcaccggccttgatatccttcagggtttcgaaaagattggatctggctctcagccgtccaaaggcttgaagcagagtggaaagcccaccctgggtcgaagctttacaaccaacttttag

SEQ ID NO: 10 - protein, 771 aa, Trichoderma reesei
MAQPPSFYASSRPLEVFTDDIFFENEAPMTSHAPMPNPTRPIRRPLSNSSSNVILEPAVSQNSNKISPYKPKTAAPRAPLKPSHGLNKFNGISMAPPSDKAPVTDSLQKKPQLSKFKTGLQKPALASPHDAFFGKENVQPQIFPAPPAINISMEQYYQDSNGKRGLMEAAPIKDSRVSSKKPKAEEQVLPPHDSFPPITDDGTKPPHSYAQLIGMAILRSSKRRLTLAQIYKWISDNYSFYNPNDAGWQNSIRHNLSLHKNFIKIERPKDDPGKGNYWGIEPGTEFQFLKEKPTRKAVPTAENLPVMSTRLEPSRPTPALMPEPCLPPPAPIHHHQHHHHQQHQPHATSLPPLPTSQATLSMPLEPSSDATILLSDNAPAEDLADKGPDNELPLESSFFSPLPPALNSTPPVPRPERRNGTPPPVSRNPASSATRTHKRKFASMDDSGYISSLESSAMRPNAARSALFTSEADRHRIKRGRAEEEIARLRGSSPFSPTKSRSLSSYGLTSSSPFRQSLDNQMPPPLTPVVKKVKPIAQPPPSVSPNTNLRIHRAKVRHMLQSPLRRVANLSNEDMLPWSPAFRLDDSIFTFDTPNASSNLPDFDIFQDLPVDDENVFDSIGAADAGSPIKRSAKRARLDRSISSSAIGEMSASKRLLTSAPLLKAPDTPSQFLETPSKVFEGLSSPSKILQGSPIRGNSPSKFVSLIELPGDADWPSLHLDTGDFASGDTSDFTGLDILQGFEKIGSGSQPSKGLKQSGKPTLGRSFTTNF

SEQ ID NO: 11 - DNA, 1906 bp, Trichoderma reesei
atgccgtcaaatcaagcgagcatcgagctggaggaaatcttcaaggagctcggcatctcgcagtatcttgacgcctttgttgaacatggctttgacacttgggacaccatcctcgatattcaagagtctgatctgtgggtaaactgctgcggtggcgtgtgatgtagtctcagcaaggctaactagactcccagcgatgctttaggggtcaagctcggtcatcggagagtaagtattcgagtcgcagagccgtgagaggagtactgaccgcggacatcatcagaaacttcaacggcgaatcgccaacgcaaggggaatcgaccccagcatctcattgtcgtcggtacaagctgccatcgaagatgaaaagtatgacgggtctcatagaaaacgagaagccccctgccatgctggaaatggcggaagcagtactgcgaagcgcaaataccgccggcatcctaaggtatagctcaatcagtcaagcggccacccctcgggccagattgctaatgccttctgtccttagcccgacgaaaacgcgcctccacggcccatgagcgcatatgtgctcttttcgaatagtgagttccaatgttgtgccccccgctgccctttgtgttggcaagatcgagactatttggctgaccaaacacggtagaactcagagaggatttgaaaaaggacccttctctctcctttgtagcaatagccaagctcgtagggggcaattggcaaaacctcccattgcaagagagagagtattatgaagcccaggcgagagcggataaggagcgctattatcgagagatggcactctacaagcagactccgcaatatcaagagtacatgaagtacctccaagacttcaacgagaagcaagccaaactcaggcgaggtccgtggttccttgaatcggtattgtagccgtggggtgggtacagttgaagctgatgtttatttctgtgccggctagaccaagagaatgccaaacgaacagccatgcgcggctcgacctcaacagcgagtggcagcggcagtagcagcgagagaatgcgagaaagcgagtctcgcgagccccctagccttcgacataacagctcgaattcgatgccctctccttcaaggacccatcatatcacggcgggcaccgggacgtatgcagggctgacacaaccaaatggcgacatacccacacaaagcgaatcgcgctctcgacgacgatattcagcagagaaggagagggaacaccagaccgacaccccgcagcaagttctgccctcagttttcgatctgctggccgatagacaaggtcagccgagcgccccagtgacaaacaactccggactcatgcaagggctggcgccagccgatcatggcaggcggtctatatcccacggcacaccctcgtggccagtcgactcaaggccgtcccttcatcacgagccatcttctgctggaagcagtataccgactgcgtcgtcaagcagtctgagtagtagattaagcgctggccatatgccaatccatgcgctcttatcgtcggatgaaggtcaaccgccccagcgcattgacgatattccagcatatacgtcccactatgtttccagtccaattgagcagaagaggccgtccatggagtatcaggggcctaagggatacggtatccatccccccaagtgcccgagtatttggagaaaagctgctaacatgaggtgccaaaggcttccagaccgcttcctcggcgttccagcatatgagattcgaggagacaagcaacggggatgtgctgatgacttcggcaaacgaaacgccgcctagcgcctctcccatgagcgttagcgggcttgatggggtgaacgctctattaaaagctggcgaaatcgtcggccatcctcgccaaatctaa

SEQ ID NO: 12 - protein, 503 aa, Trichoderma reesei
MPSNQASIELEEIFKELGISQYLDAFVEHGFDTWDTILDIQESDLDALGVKLGHRRKLQRRIANARGIDPSISLSSVQAAIEDEKYDGSHRKREAPCHAGNGGSSTAKRKYRRHPKPDENAPPRPMSAYVLFSNKLREDLKKDPSLSFVAIAKLVGGNWQNLPLQEREYYEAQARADKERYYREMALYKQTPQYQEYMKYLQDFNEKQAKLRRDQENAKRTAMRGSTSTASGSGSSSERMRESESREPPSLRHNSSNSMPSPSRTHHITAGTGTYAGLTQPNGDIPTQSESRSRRRYSAEKEREHQTDTPQQVLPSVFDLLADRQGQPSAPVTNNSGLMQGLAPADHGRRSISHGTPSWPVDSRPSLHHEPSSAGSSIPTASSSSLSSRLSAGHMPIHALLSSDEGQPPQRIDDIPAYTSHYVSSPIEQKRPSMEYQGPKGYGFQTASSAFQHMRFEETSNGDVLMTSANETPPSASPMSVSGLDGVNALLKAGEIVGHPRQI

SEQ ID NO: 13 - DNA, 3787 bp, Trichoderma reesei
atggaggctcaagttgttcccgctctgcaaagagctgctgagattggaaccctctacgatgcaaagagagatgagttcctcagcgcttcactattccctcaaggtctgccttctagtcatgtgtcgacacagccaacccccggcacggaaatcaccgtctccagatgcgcttcctatggagacgtgttcagagatctccgtatagacgaaacgactgcggtcaacattctctccgggacactgcgtctccaaggagcagccatgttcctccgagacttcgaagttggcggaatggtgcgcgctgccatcatccatcgactctctacagtgcagcagaccctcaccagcctaggcgacaccttcaaatcagctggcgatttggcccccttgaagacggaagactgcactcacgtcgtgatcggcatcaaatggggcctgcagactgttgtggccgccagctgccccacgccaggcagaacaagttctcttaaagctgaagaggcgacctttgaacgggatctggaggttttgctgactgccattgagtccgcctcctttacgccgcagacggcctctcccaggctgagctctcaactggagctctgcaatgagttcaccatctacagcgatgcttttgaacaggagggtctaccgatgcaaagcgtatccgaagtatgcgacttcatccgcatcgtacccgaccatcttcggcaagcaaacagcggcaaggggttcccagtcgcatatacgttgatgcccgtcacgacgctcaaatacatcatgcctctgccgcgcgagcttccgtacaactcaacgttcatcaggcccgattatctggccaacttcatccatgtcttcgacgatatcgtagcttccgcaggcaggcttcgcaagtatcacaactacttggctggccacatcatgtatgtctccaaaagtcacgttgatgaggttttggcctcgattgttctcctggaggagggcaagagccggctcaccgagcaactgttcaactgcttgtcggacgtgcgccatggcaggcaggatccctcagttctcttcgaccttcatcgtcatgcggccctcagcgagcccacaccaaggcagctctctgaccttgtcggacaggagaccgagaagctcgacttcattgcaagtgtcatctctcagggggccaactacataggccacaacggactttctctcgaaagggtccaaaaggcctacaaggacgctcaattctacgtctttcattttgattccgcctctatgaaaggcgatcaatactggagtgacaacctggccttgttgctcgaactcgtcggacagcgagatgagcgcctacccgtcttcatcatggaccacgacgcgataggctccgacgcctgtgttggcaattcacgtattgcacagtacaagggcggcgaagtatgcacaatcgatatgttggagtatcgccagtttgtctcctcgaaatgctttgctcgatgctccggcgcaacacttgatacgagcaggtgtcaaagacctgtcaaaagacgctttgtcaaagttccctgcccgagtcctgggtgtgattccggccattctcatcagtggacttgtccgaagtgctttgcaaccatcgagtatggcttcagcgacgacttcttctactgcgagtgcggccgcggactctttagtgactacgagttcaagtgcaacgatgcaaagcacgacgctggcttcaagacgtatgatgtggaacagcttcgaaaactgctgggcaagttggatcaagatgactacatcaacatcctcattctgggagagaccggagtgggcaagtcgaccttcatcaatgcctttgtcaactacctgtcttattccacgctggacgaggccaaggatgccgactcgctcgcttctgtcattccttgctcgttctctcttcagacgatggacagggaaaacccggagttgggcattcaagaattcaacgtcaaagttggtggacgtgacgatgaggctgacggatcgacgggaaactcggcaacccagaagtcggccgtgtatcccgtcatgatcggggcaaaggcgtatcgcctgattgacactcctggcattggagacaccagaggcttgtcttacgacaaggagaacatggccgatattctcaagaccatcagctcttatgacaagctgcacggcatcctggtgcttgtcaagtcgaacaatgcacgcctaaccgtcttgttcaaattctgcctcaaggagctcctcacgcatctacaccggagtgctaccaagaacatggcttttggcttcaccaacactcgcatctccaattatgcccctggcgatacgttcaagccgctcaaggctctccttgacgagcagtcggatattcccatctctctctcgacggctacaacctactgttttgactcggaaagcttccgctaccttgctgcttataagcagggcatacccatggacaacgaggaagagtttcgtcgaagctgggaccactcaagcaaggaggcccatagactcctcaactactttgcgtctactcagccacaccctgtcaccagcacactgagcctgaacggggcacgaaaggtgatcctcgagctgacaaagcccatggccagcatctcggactcgatcaagaagaacatcaagttgtgcgaagacaagaagatggagctgagtgatacacagttaacagcaaccaagcttcgcaggaggctgcgtctggagagaatccagttcaaggccgtcaagctggacaggcctcgtacggtctgcaaaaaccccgactgttgcgatttcaaagacagcggtcttgatgatggagttgtcgtcaccatatacaagacgcactgccacgctgagtgctatcttagcaatgtcacccaagatgtcgttgctgatcccggcctcatcaactgctgggcattcaacggcacgagtaattgtcaagtttgccaacaccgatggcaagagcacttgcacgtcctctacgagctcttcgagaccaaggtccaggttacagacaaagagatagagaggcagctcaaggccaacgcggacgacgtaaccctgcgccaactggcgattactacacttgaccgagacattgcagagttcaacacagagttggatgaaatccgtcgtgcgtctgcccgattctgtctgttcttgagagcaaacgccatcaccgtcatcaacgacgccacgctggactacctggacatgctcattcaggacgagcagggcactattgaggccggcagacaaagaggattctgcgttgacgccaacaagaagcgtcttcaagcgctcagagaggatagaaagatccatctggagctcgtggaaaccttcaagcaaaacatggctcatccgacctgtcccgaggacatgctgctcgacgagcaaggagtcgacgccctcgtcaagaaactgtacaatctgaagcatttcggcgccaacttgaggcacatcaagtatgtcattgactcctccatcgaagacacgtaccgtgagcggccttatcgcgtgcccactttcaactttcgcaagcaagtcgcaagtcaccggacgccacgccatacgagccagcgagggacatggcagggttcgggtgaagggtatttccatcagacacagcactctgtgggaaacgcggttggatttgaaggtcatgggtatccgccgctttcgtacgcggatgctacaaagtcgatatctcatcggttcaagtcgttttgggagaggagcattgcacgaggacgggtga

SEQ ID NO: 14 - protein, 1243 aa, Trichoderma reesei
MEAQVVPALQRAAEIGTLYDAKRDEFLSASLFPQGLPSSHVSTQPTPGTEITVSRCASYGDVFRDLRIDETTAVNILSGTLRLQGAAMFLRDFEVGGMVRAAIIHRLSTVQQTLTSLGDTFKSAGDLAPLKTEDCTHVVIGIKWGLQTVVAASCPTPGRTSSLKAEEATFERDLEVLLTAIESASFTPQTASPRLSSQLELCNEFTIYSDAFEQEGLPMQSVSEVCDFIRIVPDHLRQANSGKGFPVAYTLMPVTTLKYIMPLPRELPYNSTFIRPDYLANFIHVFDDIVASAGRLRKYHNYLAGHIMYVSKSHVDEVLASIVLLEEGKSRLTEQLFNCLSDVRHGRQDPSVLFDLHRHAALSEPTPRQLSDLVGQETEKLDFIASVISQGANYIGHNGLSLERVQKAYKDAQFYVFHFDSASMKGDQYWSDNLALLLELVGQRDERLPVFIMDHDAIGSDACVGNSRIAQYKGGEVCTIDMLEYRQFVSSKCFARCSGATLDTSRCQRPVKRRFVKVPCPSPGCDSGHSHQWTCPKCFATIEYGFSDDFFYCECGRGLFSDYEFKCNDAKHDAGFKTYDVEQLRKLLGKLDQDDYINILILGETGVGKSTFINAFVNYLSYSTLDEAKDADSLASVIPCSFSLQTMDRENPELGIQEFNVKVGGRDDEADGSTGNSATQKSAVYPVMIGAKAYRLIDTPGIGDTRGLSYDKENMADILKTISSYDKLHGILVLVKSNNARLTVLFKFCLKELLTHLHRSATKNMAFGFTNTRISNYAPGDTFKPLKALLDEQSDIPISLSTATTYCFDSESFRYLAAYKQGIPMDNEEEFRRSWDHSSKEAHRLLNYFASTQPHPVTSTLSLNGARKVILELTKPMASISDSIKKNIKLCEDKKMELSDTQLTATKLRRRLRLERIQFKAVKLDRPRTVCKNPDCCDFKDSGLDDGVVVTIYKTHCHAECYLSNVTQDVVADPGLINCWAFNGTSNCQVCQHRWQEHLHVLYELFETKVQVTDKEIERQLKANADDVTLRQLAITTLDRDIAEFNTELDEIRRASARFCLFLRANAITVINDATLDYLDMLIQDEQGTIEAGRQRGFCVDANKKRLQALREDRKIHLELVETFKQNMAHPTCPEDMLLDEQGVDALVKKLYNLKHFGANLRHIKYVIDSSIEDTYRERPYRVPTFNFRKQVASHRTPRHTSQRGTWQGSGEGYFHQTQHSVGNAVGFEGHGRFGRGALHEDG

SEQ ID NO: 15 - DNA, 1779 bp, Trichoderma reesei
atggcgcagtacggcccctatgttcgactctggggcatccgcccgtcacaactccctgcagagaccgcgtcagctcaagacctcgtcccattcctccagtccatcctgagcgaagcagttcctttcatcagctcagtgcccctacaatcttcctcctcgtcatcatcatcatcctcctcctcatcgggcaaatcgaacaagtccagccaaggtggtgctggcctagacggcccctggaagcccaagggatgcagggcgttccctcactccgccgcgccggtgcacatgtttgagcgggtcgtctcggccgaggacctcaagctcgcggccaagagcgacaatctcccgcaggcgtcttcttcttccggcgccgcgcccaagttccggccggaaacgtggagcctccggcgcagcgtccacgagggcgccaggaaggccggcaccgcggactgggacgagtggaagcgctgcttcaaggacgagcacgccgacgccgagatgaagtttacgccctcggtcatggaccacgcgctcaagaagcagtgggattgcacgggcgccgaggtcacgctcgggggcgacacgtacgaggacctgacgctcaagctggaggagtcgacgcacaagatgcccgcgccgctcaacaaccgcgtcttccccgtgctgcagctgacggcggcggtgaggggccggcgcgagttcatcgtcgttcagatcgcggccgtgcccgagtcttccaaggcggaagagcagaggccggatggggcggtcaggggcgcgtacacgagcatcgagcgcttccgggaggtggacgagcagggcaacgtcgagtggctgatggggacggtgtcggatgcgaggggcgtgctgccggcgtggatccagaagatggcggttccggggcagattgcaaaggacgttgacatgtttctggagtggattgcaaaggagagggagggcaacccccccaggcccttcgctgacgcagacgccgtcgcccgcataaccgtcgaactcaacgtccgcaccgtcagcgcccagacagagcgcctggaagaggagctcgccgccctcgcggagtacatcgaggcggacaaggagtttcgggagaagcacgacaagcggctgcagaacctgtgcaacgagatcctcgcggtgaagcagcgcgtggtcgagattcaggggcccgagtggaacgagggcggcggcggcggcggcgagaaggccgaggcgggacagaaggaggtggacgaggcggttgagcggctgaggagggagatgggcgagatgaggggcttggtcagtgatatgtcgagtgcgctggacaagctcccgacggcggcggaggcagaggctttggtgaggcgctctcaggctccggcttcagcgagcaacggggacgcaacgcaagcacctgctatagataaggcctcgggaaccaaagaggcacgatccatcaagcaacgcatcgaagacaccatcgcctccacccgccggtggaacagcgaccacaagacgaccaagctcaccgacgccgtcttcatcgcgaattacctcaagcagcagtccaagcgcgatccccagatggctgtctacatgcagaggacgatccagaagcacgttcagaacagcggtaggcgtccgcggacaagggcgcgtccaaagagcctcgagcagttttgcaggatgctggtgtggaaggacgtgcttgatacggcggaaattgtgctcgtgagggattggagaaggactgcgagggcgttggaggagaggggttga

SEQ ID NO: 16 - protein, 592 aa, Trichoderma reesei
MAQYGPYVRLWGIRPSQLPAETASAQDLVPFLQSILSEAVPFISSVPLQSSSSSSSSSSSSSGKSNKSSQGGAGLDGPWKPKGCRAFPHSAAPVHMFERVVSAEDLKLAAKSDNLPQASSSSGAAPKFRPETWSLRRSVHEGARKAGTADWDEWKRCFKDEHADAEMKFTPSVMDHALKKQWDCTGAEVTLGGDTYEDLTLKLEESTHKMPAPLNNRVFPVLQLTAAVRGRREFIVVQIAAVPESSKAEEQRPDGAVRGAYTSIERFREVDEQGNVEWLMGTVSDARGVLPAWIQKMAVPGQIAKDVDMFLEWIAKEREGNPPRPFADADAVARITVELNVRTVSAQTERLEEELAALAEYIEADKEFREKHDKRLQNLCNEILAVKQRVVEIQGPEWNEGGGGGGEKAEAGQKEVDEAVERLRREMGEMRGLVSDMSSALDKLPTAAEAEALVRRSQAPASASNGDATQAPAIDKASGTKEARSIKQRIEDTIASTRRWNSDHKTTKLTDAVFIANYLKQQSKRDPQMAVYMQRTIQKHVQNSGRRPRTRARPKSLEQFCRMLVWKDVLDTAEIVLVRDWRRTARALEERG

SEQ ID NO: 17 - DNA, 2658 bp, Trichoderma reesei
atgtccaagaccttcttcggcaatgtcaagagtgtcttgagcggcgacactctggtcctcaccagcgccaacaatcccgcggccgaacgcacattttcccttgcctacgtttcggcccctcacctcaagcgagagggagacgagccatttgccttccaatcccgcgaataccttcgaaatctcgtggtgggcaagcccgttcaatgcacagtcctctataccattcccacgaccggcagggagtttggcactgctcagctcaaggacggaactctcctgcccgatgagcttgtcaaggccggttgggtcaaggtccgtgaggacgctggccgcaaggaagagtcggaggagcttctcgataggctagagaagcttcgtgctctggaatccgaggccaagggcgcctccaaggggctctggtcgggcaccgacggcaccatcgaggtacagaatgacctgggtggccccgagttcctgacgcagtggaagggcaagaccgtggatggcatcgtcgagagggtgttgagcggtgatcgtctgttggtccgactgctgctgtccgataagaaacacgttcagcccctgaccctccttgccggtatccggactccgtccaccgagcgcacgcttccttccacgggtgctacgcagcctgccgaggaatacggaaacgaggccaaagcgttcgttgagtcaagactgcttcagcgacaggtcaaggtcgagattgtcggcgccagcgcccagggacagctcattgccagcgtcatccacccccgaggcaacattgccgagttcctgcttcaagagggactggccaggtgtaacgacttccactccactatgcttggagagaagatggcacctctgagagccgccgagaagcaggctcaggccaagaagctccgcctacacaggcatcacgtggccaaggcagacgctggaaccaacgagatggttgtcaccaagatcattggcgctgatacgatcatggtcaagggcaagaacgacaacacggagaagaggatcagcttcagcagcatccgagggcctcgcaccaacgagccctcggagagccccttcagagacgaggccaaggagtttgtgagatcaaggctcatcggcaagcacgtcaaggtgagcgtcgacggaaccaagcccgcttccgagggcttcgaggccagggatgtggccaccgtcaccgagaagggaaagaacattggtcttgcgctggtggaagccggactggcctctgtcattcgacaccggaaagatgataccgacagggcgcccaactatgatgagctgcttgcggctcaggagaaggccaaggaggagaagaagggcatgtggtccggcaagccccaaaaggccaagcagtacctggacctctcagaaaacacacaaaaggcaaagattatgctcgccacgctgcagcgacagaagaaggtgcctgccattgtcgacttctgcaaggccggctctcgattcaccattctcatcccgcgcgagaacgtcaagctgacgttggttctgggcggcatccgcgcccccagggcccctcgcgccgatggacaaggcggcgagccgtttggcaaggaggccctcgacctggccaaccgacgatgcaaccagcgagactgcgaggtggacatccacgacatggacaaggtcggcggcttcattggcagtctgtacattggccgcgagaactttgccaaggtgctggtcgaggagggcctggcttcggtgcacgcctactctgccgaaaagtcgggtaacgctgccgagctgttcgcggcggagaagagggcaaaggaggccagaaagggcatgtggcatgactacgatccttcgcaggaggagaacgccgaggaggagtctggcgaggccgacgcgcccgaggccgaagtcacactggacaagaagcccgccgattaccgagacgtcatcatcaccagcattgacggcaacggcaagctcaagatccaggagattggaaagggcacggccgcgctggagtccctgatgagcgacttccgcaagttccacatcgattccaagaacaacaagcccctggcggaggctcccaagactggcgagtttgtgtctgccaagttctcggccgacgaccaatggtaccgtgcgcgagtccgggccaacgaccgcacagccaagatgtccgaggtcatctacgtcgactacggcaacacggagaaggtgccctggtccagcctgcgatccctagaccagtctcagtttggtgtgcagaggctcaaggcccaagccattgacgcatcgctgtcgtttgtccagctgcccacgggcgcccattactttagcgaggccattgcctttatcgctgatctgaccgagggcaggcggctggttggcaactttgactatgtcgatagcaaggagaacgtcagctacatcacgctgtacgacaccaaggcggacggctctctgcccggacccaacgactccattaacaaggagattgtggcgagcggatacggcatggtgcccaagaagctcaagtcatgggagcgcagcaaggcttttgaatcatacctgaagcacttgagagaggttgagagccaggcgaagcaggacaggctgggcatgtgggagtatggtgacattactgaggactaa

SEQ ID NO: 18 - protein, 885 aa, Trichoderma reesei
MSKTFFGNVKSVLSGDTLVLTSANNPAAERTFSLAYVSAPHLKREGDEPFAFQSREYLRNLVVGKPVQCTVLYTIPTTGREFGTAQLKDGTLLPDELVKAGWVKVREDAGRKEESEELLDRLEKLRALESEAKGASKGLWSGTDGTIEVQNDLGGPEFLTQWKGKTVDGIVERVLSGDRLLVRLLLSDKKHVQPLTLLAGIRTPSTERTLPSTGATQPAEEYGNEAKAFVESRLLQRQVKVEIVGASAQGQLIASVIHPRGNIAEFLLQEGLARCNDFHSTMLGEKMAPLRAAEKQAQAKKLRLHRHHVAKADAGTNEMVVTKIIGADTIMVKGKNDNTEKRISFSSIRGPRTNEPSESPFRDEAKEFVRSRLIGKHVKVSVDGTKPASEGFEARDVATVTEKGKNIGLALVEAGLASVIRHRKDDTDRAPNYDELLAAQEKAKEEKKGMWSGKPQKAKQYLDLSENTQKAKIMLATLQRQKKVPAIVDFCKAGSRFTILIPRENVKLTLVLGGIRAPRAPRADGQGGEPFGKEALDLANRRCNQRDCEVDIHDMDKVGGFIGSLYIGRENFAKVLVEEGLASVHAYSAEKSGNAAELFAAEKRAKEARKGMWHDYDPSQEENAEEESGEADAPEAEVTLDKKPADYRDVIITSIDGNGKLKIQEIGKGTAALESLMSDFRKFHIDSKNNKPLAEAPKTGEFVSAKFSADDQWYRARVRANDRTAKMSEVIYVDYGNTEKVPWSSLRSLDQSQFGVQRLKAQAIDASLSFVQLPTGAHYFSEAIAFIADLTEGRRLVGNFDYVDSKENVSYITLYDTKADGSLPGPNDSINKEIVASGYGMVPKKLKSWERSKAFESYLKHLREVESQAKQDRLGMWEYGDITED

SEQ ID NO: 19 - DNA, 725 bp, Trichoderma reesei
atgggtgtcggaacattctttcactacttcggcacggtgctgctgcttgccgccacggcgctcctcatcgtcgtgtccgtgactgcgcccgtcgtcaacgacctctctctcctgaagatcaacttcagcggcagcagctctgtcgacagaataacatttggtacctttggttactgcatccaaaatgcaaagtacgtcttggttcatctactcattactctcaaacactaccatatgagaaacacaatagctaacatctacaaagcggacacgatgcatgcacccactctcacatcggctacgagggcaccgacgccttgacccaggtcacctccctcaacttctcaaagtccgaccgcgatggcttcaggattctcacaaaggtcatgatcctgcaccccatcgccgccggcctcaccttcttggccttcctcctctgcctgggcaccagcttcctcggctcctttgtcgcgtccttcttctccttcctggcctttgtcgccaccgtcatcgccatggcctgcgactttgccggcttcgagatcatcaagcacgccgtcaacaccaggggccctgccaacgtcgaggcccactggggacctgccgtctggtgcattctggctgctgcggcgctgacgctgattgcgaccatcttggtgtttgtcacttgctgtgccggtcgtgtcaagaaccgccgcactcgtcgcagcaagtccgagtactaa

SEQ ID NO: 20 - protein, 216 aa, Trichoderma reesei
MGVGTFFHYFGTVLLLAATALLIVVSVTAPVVNDLSLLKINFSGSSSVDRITFGTFGYCIQNANGHDACTHSHIGYEGTDALTQVTSLNFSKSDRDGFRILTKVMILHPIAAGLTFLAFLLCLGTSFLGSFVASFFSFLAFVATVIAMACDFAGFEIIKHAVNTRGPANVEAHWGPAVWCILAAAALTLIATILVFVTCCAGRVKNRRTRRSKSEY

SEQ ID NO: 21 - DNA, 606 bp, Trichoderma reesei
atggctcccaagattgccatcgtctactactccatgtacggccacatccggcagctcgccgaggctgagaagaagggcgccgaggccgccggcgcccaggtcgacatctaccagatccccgagaccctccccgacgacgtcctgggcaagatgcacgcccccgccaagcccaccgacgtccccgtcctcgaggaccccgccaccctcacccagtacgacggcttcctgtttggcattcccactcgttatggaaactttcccgcccagtggaaggccttctgggacaagaccggcggcatctgggcctcgggcggctactggggcaagaaggccggcctcttcatctccaccggcacccccggcggcggccaggagtccaccgccatcgccgccctgtccaccctggcgcaccacggcatcatctacgtgcccctcggctacgcaaagaccttccccgacatcaccaacctcgaagaggtccacggcggctccccctggggcgccggcacctacgccggccccaccggcgcccgccagcccaccgccctcgagctcaagatcgccaccgcccagggcgaggcctttgcccaggccctctccggctga

SEQ ID NO: 22 - protein, 108 aa, Trichoderma reesei
MSGKGGQGGDGGGLLAAAGGAGGDEEAGLLAPVAARGPDAAGLVPEGLPLGGKVSITSGNAKQEAVVLGEGGGVLEDGDVGGLGGGVHLAQDVVGEASASCRMWPYME

SEQ ID NO: 23 - DNA, 2838 bp, Trichoderma reesei
atgacagacgacccggatcgcgcgtgtccggctccgacaatcgccgaggacgcggttgcgaagagcgcgatcacgcaggcggccggcgtcgaggttccagctccagctccagctacagctccagctacagctcctacagctcctacagctcctacagctcctacagctcctacagctcccacagctcctacagctccaactacggctccaaatgcggcatcagctccaacaccggctccagctctagctgagggcaccagtgccgagcctcaggaagcgcgctccaggaagaagcggctgcagctgagctgcggcgagtgtcgcaggaaaaaggtaacggtaacgcggcagaggagaagagcaaaccgatgagagatcgcgtctggcaagttcgtgttctaaccagcaacccatcgccagctctcctgcgacagaggccggccgtgccgcagatgcgtcaggacgggcagagcagaccaatgcgagtttgagaccaacccgcgttcagcgctgctgcccatcgaccagggtgcgcagctggaacagatcaagtccgaccaggccgaacttcagagcctgcgggctgagatcttccagctcaaggagctgctctcacggcctctggcgcctcctccgcctccaagtcaccctcagcctcagcctcagccccagccccagctacatcctcaccatggcagaggctccctcacggaagactcgcgcgtcagcgacttctcctccaaaagaagcagtgagatggaatccaaggacattgtgaccatcagccggattcccaacagctttagtgacggcccgcctgatcccagggaaaaatcgccacgaggatactacagacggcacgccctgctgaggttcttttcagaggtaatctcgcgcctgactctaccgtgcttaccgttgattgttttaacccggcgcaacagattccacagctgtttccattcatcaaggagattgccgaccagtggctgaagccctatggaatctacatcaagaagaacaaggtcgccaaagatgagagctggaggatgaagatggcagccaacgagcagcctctgattgaaggcctcttgccgccaaagcatgacacggacgttctgattgcgatataccttgatcatttcgagcaactgcatcgcattgtccacatcccgaccttcaacagggagtatatcaacttctggacgcccggccgaccgcggtacccgacaatggcagcaatgatactcgccatgatctccgtgtcggtctgtgcctccgtccaccttgttggctcttcgccagtccctgccagctatcgaaaaatggctgagacgtggatttccgccatcgatgactggctgcgactgcaaagcacaaagcaccgcaagctcgtgcactaccaggtggccagcttgatgtacattgccaagcggatgaacctggtagggaagaagaggttctggaaagatacgggctccatgatccaggacgcaatcatggatggcttaaatcgcgatccttcactggcagataccttcttcatcagggagatgaagcgacggctgtggtacaccgtccgagagctggagcttcagaactcgtttgaatgcggcttgcccactcttcttcatacgattgagtcgaatgtcacagcgccgctcaaccttgctgatgaagattttgacgacacaacgaagctgacgcccatgtcgaggccgtccaattcctacacggagtcttcgtaccagttccacagcttccgaagctgggagttgcgtctcgagctgtcacgacgtcagtttggttccggagttttcaggcctctcgattatgacgacgttttgcggtacacacacgagatcacacgagcattgaacgatctccctccgtggagcaatgacaggactgacagcaaaactgatggacgggtgctcctgtcgtactcgatgctggagtttcagctcaaggagtgtctcctggccatccatcggccatatgtggacagggaaggcggcaaataccccatctcggaaacgatctgttaccagacagcgcgagaaatactgctttccaacatacagctcgcagccttgggagttcaaagtctgacacaactccgcgaagaccttgcaattgcttcgctgcacatgactcgccttacactcatgcaacaccaaggtgggctcttctttcttcttttgtatatctaaggaggtcttctgacttgtagaaggttccaacagcatcatcatggtcaatgccttgtcgactgtcgacctgctggagcagtgcctcccggccattgaggataaatatctgcggttcgccgacccttggagctttttcatgatgtgtacagccattatgttgatcaaaatacacctcggaaaagagactcgccaaactgccaaggcggcttgtgcacgcagatttctggatctgtactacaagagtgtttggatgcatcacccctctgacctcgctcaacagcaggcaatttctcgggatatcgccaaccaaacaagtgtgagttctatgataaccgttcaggattacgctatttcttctggcacagccttcggggatcaatctaagcttacttttacgtctatcctaggctcccgctgcctgtgctccagctccttcagacggaagcgtccttcccacctcggtgtggcttgaaaacagctatcccgatgtatgtccaaaggcttgctaataatgctgcatgtactaatcctcccacagattgggaccgatccgttcgacctatccgtcgagatggacgtctgggacgaggcgggatttcctctgccgggatttcccaactgctag

SEQ ID NO: 24 - protein, 832 aa, Trichoderma reesei
MTDDPDRACPAPTIAEDAVAKSAITQAAGVEVPAPAPATAPATAPTAPTAPTAPTAPTAPTAPTAPTTAPNAASAPTPAPALAEGTSAEPQEARSRKKRLQLSCGECRRKKLSCDRGRPCRRCVRTGRADQCEFETNPRSALLPIDQGAQLEQIKSDQAELQSLRAEIFQLKELLSRPLAPPPPPSHPQPQPQPQPQLHPHHGRGSLTEDSRVSDFSSKRSSEMESKDIVTISRIPNSFSDGPPDPREKSPRGYYRRHALLRFFSEQIPQLFPFIKEIADQWLKPYGIYIKKNKVAKDESWRMKMAANEQPLIEGLLPPKHDTDVLIAIYLDHFEQLHRIVHIPTFNREYINFWTPGRPRYPTMAAMILAMISVSVCASVHLVGSSPVPASYRKMAETWISAIDDWLRLQSTKHRKLVHYQVASLMYIAKRMNLVGKKRFWKDTGSMIQDAIMDGLNRDPSLADTFFIREMKRRLWYTVRELELQNSFECGLPTLLHTIESNVTAPLNLADEDFDDTTKLTPMSRPSNSYTESSYQFHSFRSWELRLELSRRQFGSGVFRPLDYDDVLRYTHEITRALNDLPPWSNDRTDSKTDGRVLLSYSMLEFQLKECLLAIHRPYVDREGGKYPISETICYQTAREILLSNIQLAALGVQSLTQLREDLAIASLHMTRLTLMQHQGSNSIIMVNALSTVDLLEQCLPAIEDKYLRFADPWSFFMMCTAIMLIKIHLGKETRQTAKAACARRFLDLYYKSVWMHHPSDLAQQQAISRDIANQTSAPAACAPAPSDGSVLPTSVWLENSYPDIGTDPFDLSVEMDVWDEAGFPLPGFPNC

SEQ ID NO: 25 - DNA, 2626 bp, Trichoderma reesei
atgtcgacggctaaagcgaccaagaagagcgcgttttcctgcgagccatgtcgccgccgcaaggtaagcctggctgcttggagatcgtgttattggcgtgtctctggctttgctgggagggatggagagagtgtaagagaggcatcttggagctgactctgctggtggcgttaggtcaagtgcggcggcgagcagcccatgtgtcaacgctgcgttgctcgcaacgatgattgcgtctacaaattgtgtgacttgatatctcactccgggcacatctacatctgcctagtagcgtctgatcttggcggctgacacccatctgcaggaacccgacgctgtcctacacgcaacggctggaggatcgcatcaaagagctggaggagcagcttgcacaagtcacggctgcaacaccgccaccggcggccattggcagcaaatcccctccatcaagtcatccaagccctacagcctcggggagcggtcagcaagacacccggcagcagatcgatgacagcatctcccggagcttccgcggcctcaagatagaccaaaagggcggcgtcacctaccatgggaccacgagcttcttccatctccccagtgaccgtagctctgcggcactaactgccgacatgcacgcagccgcgactgacattgaaactcagcggagagagcgactagtttcgaacgcgtggcagcagcgcttcttggaggagactgctggggttcccgtacgtgtccaagtgtggtggtcgtcgcttgacagctcaagtctcatgccttaactctctcgcaggaaccctttcagacgcttctgaatgttcactggtgttggatacagcccctcttcaacttcatctaccgaccagcatttacccgtacgaaacccttgtagcttgtcatgacttttcccgccccctttcttctcttccctgatatttaccaatcatgtaggcgatatgcagtccatggggccgtactactcacatacgcttctcaatgctgttctctcccactccatacgctgggcgaagagcgaccccaagacaaagcagattctcgatgagtcgtacgacggaggcgccgtctttggcaagcacgcacgcagcatgcttttcgacgagctcagcaggggagtatgcacgatcccgacggtccaaacactacttctcctaagcgcacaggaatgcagccttgggaacacaacccaagcctggacgtatagtggtctggcgtttcggctgattgaccacttggggatatgtgtggatggagatcgatatatcaactcggtccacttcaccgacgaagacttggaaatccggcatcgagtcttttggtcgtgctatttctgggacaagatcatcagtctgtatcttggtcgatcgccatcactcaagcacacgacggtctctccccctcagattatgtgtaagtctgattgtcgatactcatctcttgtcgttgtcgctcacattggcggtggttggttggtcgttgatgctgacggctgtttcttataagtcgacgactctgctgaaaacgagctgtgggttcccttcggggcgcctccactccaaaccaccccctggaagtacccacctgctactgctcattcggcttcttgctttctgagcatgtgccggctgtcagtcatcttcaacgagatattgattcatatgtacgacccgctcatgcagaacaccgagtccgaggtgctcgagtgcctgacgacccaggagccggccctgcagcaatggtgggatgagcttgctccatatctcaagctcgagcctatggctctgcccagcttggccccaccgtctcacatcgtcacgaccaagtaagttcttgccgtcccaagtgccgaacgccgttctccgttctccgttaaccggtccaaatagctgcctctatcacacgttcaaggtgctattgtaccgtccaatgctcacccggagaatactggagggcgatgttgcgtcgaaccagcgctacctcgtcgagtgcgtatcgtccgccacggccatcatcgctatttttgatctcttctgccgaacctttaccatgaactactgtgtgctgtcgctggcttacagcgtctacattgcttcgtcgatattcctgctacaggttcaggcggctccgggcgacggacaagctctagggcggctcaactactgtatgcaggccttgaagcaagtcaagactttcagcccgggtaagtcgagtattggcgcgactttgcttcttttgtttttctgcttgtcgttcggctcgatcctgacgttttgtagtcatcggcagcgctatcagcgcgctgaatagggaactctcggctgtcggcatctcgctggacgtgccccagcaccagcagccacccccaatccccgtcactgagtttcctccaatagccccaggatttccggccgtcgatgtgcccgtcgatgcgtcgtcttcgcagcctcagcctgctcatccgctgtttcaggttccctcaacccacagctatggtgcccattcaatggccatggacccaggcatctttgaggccatgtcgtccctggagccactcagcgtccgagttggggccctttcccctacggaaaatcaggactcgttttga

SEQ ID NO: 26 - protein, 686 aa, Trichoderma reesei
MSTAKATKKSAFSCEPCRRRKVKCGGEQPMCQRCVARNDDCVYKLNPTLSYTQRLEDRIKELEEQLAQVTAATPPPAAIGSKSPPSSHPSPTASGSGQQDTRQQIDDSISRSFRGLKIDQKGGVTYHGTTSFFHLPSDRSSAALTADMHAAATDIETQRRERLVSNAWQQRFLEETAGVPEPFQTLLNVHWCWIQPLFNFIYRPAFTRDMQSMGPYYSHTLLNAVLSHSIRWAKSDPKTKQILDESYDGGAVFGKHARSMLFDELSRGVCTIPTVQTLLLLSAQECSLGNTTQAWTYSGLAFRLIDHLGICVDGDRYINSVHFTDEDLEIRHRVFWSCYFWDKIISLYLGRSPSLKHTTVSPPQIMFDDSAENELWVPFGAPPLQTTPWKYPPATAHSASCFLSMCRLSVIFNEILIHMYDPLMQNTESEVLECLTTQEPALQQWWDELAPYLKLEPMALPSLAPPSHIVTTNCLYHTFKVLLYRPMLTRRILEGDVASNQRYLVECVSSATAIIAIFDLFCRTFTMNYCVLSLAYSVYIASSIFLLQVQAAPGDGQALGRLNYCMQALKQVKTFSPVIGSAISALNRELSAVGISLDVPQHQQPPPIPVTEFPPIAPGFPAVDVPVDASSSQPQPAHPLFQVPSTHSYGAHSMAMDPGIFEAMSSLEPLSVRVGALSPTENQDSF

SEQ ID NO: 27 - DNA, 2337 bp, Trichoderma reesei
atggatcaacagcagaaacaacaacagccatgtcgccgccggagacccgctctctcatgtattgagtgccggcgccgcaagctcaagtgcgacagaaagagcccttgcagccgctgtgtcgcgaccgagacacagtgctcttacaccacatactaccgggataagccccgagcagggcatgggcaagatagccggatgcacgtgtcttcctcttcgatgccaagcccgtccactgtccatccggcatctactccgtcacatgtcgataccgatggtccaagtatcgttggtacagctactagcgttagagagggatcactccgtgtcggtcggacaatcgaggctcgcgccgcagcgaactcgacactcggttcagctgagtcgtcaaccgcaccagcggctgacgagatccagattccaaatcgccctcggcttctgaaccctgagctccaggatcttagaggtagggtgccagtgcacgatctatcccaggcaggccgggatatgctcactggtgagccgggtttgccggacgcccgtatcattttgaacaagacgaggattatgagatggagtcattgggttggcatagcaaaagaggtgcgtagagtatttgtgtagatcgtctactcacatctctcgtgaattagacgtgcattgatcgctaacattttggtcgtgcagttcgcccccatcatcacttgctacggagcagcttgcggctttggcgacatcaacgcctttcataacgccgagacgagagaatccattcttgaagtcagcgagctgctccagaaatgcaaaagcgtagccagaagcctcaaaaccgggcggccgagtagatgcctttcctcgctgaactttgatcttacaccccctactcgcgaggtggcagacaagatgacaaccttgtactttcagtcctttgagtcaactcatcgcatcctccatcagccgaccttctggaaagtctacgaaaggttctggagtagcccagagagcgcttcggccaagttgcggctccaagtccttctcgtcattgcaataggctccagcctggcctcgcattcgggagctaatgccggacttcgcaatatggtccatcaatggatatatgcagcagaaatctggctctcaggcccactggaaaaagaccgcctcgatatatccgggctccagatacactgtctggtgatgctggcacgacaaatcttctccattggaggcgacctcgtctggatatcctcggggtcgctcatccatcgggcaatgcagattggcttgcaccgagaccccaggcaccttccaaacatgtctgtgctgcagacgcagttacgcagacgattatgggctacgatattggagttggttgttcagtcgtccctggattcggctttaccgccgaggatctcttttgacgaatttgacacggagccgcccgccaattgcaatgaccaagatatagaagagccagtgcctgagctgaagcctcacagccgagaacggttcactggaacgtctttgcagctcattctactcgattcgttaccaacgcgtttacgtatcctcagcctcctgaacggcctgcactcggagttgtcctatctagacgtgcttgacttgagcacggatattctcgaggcatgtcgtcgttcgaacgccttttttagggataatcaagcctctggaatgactccgttccaccacaatctggttgactacctcctacgtcggttcctgattccccttcattgtccgtttgcgcacaaggcacggacgaacccgctgttacactattcgctcaaggtcagtctagatgctgccatggctatagtctatccagagccagatgaaggtttctccagtctgatggcaattggtggaggcctgttcagggagggtattcgatatgcgactagtatcatcagtctggagcttctgacacagattgaagcccactataaagatggaactttacatcgaagttcgcactccattagccacttgaagaaggcgatggaggatttgatgagtctttccctggagcgcgttcgtcaaggggagactaacgtcaaaaaccacatgttcttgtccatggtcatggctcaagcggaggctatggaacggggtgaatccggtcaacttcgcatcgcgaagagcgccagagatagtgttttgctgagcctggaactactacaaagcactcttggtgattctcctgtggcaacttctgtccaagagttcgattccgcaagcttcggcgaaagagaggactacacatatggactggggcttgacatggacttcttcttcctcaccgactcaggctttccttga

SEQ ID NO: 28 - protein, 750 aa, Trichoderma reesei
MDQQQKQQQPCRRRRPALSCIECRRRKLKCDRKSPCSRCVATETQCSYTTYYRDKPRAGHGQDSRMHVSSSSMPSPSTVHPASTPSHVDTDGPSIVGTATSVREGSLRVGRTIEARAAANSTLGSAESSTAPAADEIQIPNRPRLLNPELQDLRGRVPVHDLSQAGRDMLTGEPGLPDARIILNKTRIMRWSHWVGIAKEFAPIITCYGAACGFGDINAFHNAETRESILEVSELLQKCKSVARSLKTGRPSRCLSSLNFDLTPPTREVADKMTTLYFQSFESTHRILHQPTFWKVYERFWSSPESASAKLRLQVLLVIAIGSSLASHSGANAGLRNMVHQWIYAAEIWLSGPLEKDRLDISGLQIHCLVMLARQIFSIGGDLVWISSGSLIHRAMQIGLHRDPRHLPNMSVLQTQLRRRLWATILELVVQSSLDSALPPRISFDEFDTEPPANCNDQDIEEPVPELKPHSRERFTGTSLQLILLDSLPTRLRILSLLNGLHSELSYLDVLDLSTDILEACRRSNAFFRDNQASGMTPFHHNLVDYLLRRFLIPLHCPFAHKARTNPLLHYSLKVSLDAAMAIVYPEPDEGFSSLMAIGGGLFREGIRYATSIISLELLTQIEAHYKDGTLHRSSHSISHLKKAMEDLMSLSLERVRQGETNVKNHMFLSMVMAQAEAMERGESGQLRIAKSARDSVLLSLELLQSTLGDSPVATSVQEFDSASFGEREDYTYGLGLDMDFFFLTDSGFP

SEQ ID NO: 29 - DNA, 2311 bp, Trichoderma reesei
atggaggagccttatctcctaccaacagggaacccgctgcctcatcgcgtgagaaagatgcgcaagggaactcgaagctgcaccgaatgtatgtcctgtatagacgagttgtatctggtagcagatagacactgatagcagtctttgacaaggccgaagacgcaaaacgcgctgtgttttttctcccggtgacaccgtatgcgtgctgtgcaagtcccggggcagtcgctgtatcgaacaaggttacgaggacgccagtcttccaaatcccagatctggtgcagctcggaaaagccctaccgccgagaaggcgtctcagagtcggaatgagccatctgtagatcagccccccaagttctgtggagatgcagagacatcagatgcccttgcctcagagagagcaaaccatgctccgattgtatctctactcgtggatgtcaaggtcagttaagcaagattcaaaggccgactgcatccacatgcatgcattcacaagcgctttgaaggcgagctaatcgatggctttgcccaatgcacagctctcggcatcggcgaagggcggacaagacgagccctcaaatccaaaaccaatgccttggtccatgcagcctcttggcagcaaatctgcatatgtgtgctctacgatccgctcgatgctacccagctatgataccatcatatccgttctcactagaaacgggtcatggtgggacagcttccgctcaaaggcatatgccatttccgaggcgccttcccagacgatcgaagccttcgcgaagaggacctatacgagcagcaacccggccgatataggtgctctggccattgcatttgctcggagcttgaataagcatcgctatctgtatactctggtagatgacctcgtcatctcggacatcaacttcctgacgactatagaggggctggaatgcctcatccttctcgcaaagtcatataccgactgcggccagccaaaaagagcctggcttgtatggagaaaaggtgcatctgcaacgcagcttatggtgagtcagtccttgtttggctcagccatccggagggacgctggcctcgtctgactcgtcattagggactttgctgtggtagtaactacagccatacagaaatgaggctatggtgctcagtatatcacggcgacagattctgtagtatgctgctcggcttgccttatgtcctcaacgacaatcactaccagtctgttatcaacgcgccggggatggccccaggcttttattttgtcctccgatgcgccatcatctgtggaaagatcattgaccggaatatgactgtcggcaagccgtcattcgcgagagccatggaactagacgaagagatggaaagcgttgcgtcgtcacagccacaggactggtgggccgttacagagaccttggaccagccgctcgaacatcttgagctcaacgagctcagagagcagctattgcaacaattctacttctactgggtcaagctgttcctccatctgccattcctggtggagacgacaacgagctcgccacactacttcagcagaatggcctgcatagaggcatccaagcagattctaaagagatatcgactcttacgcaccagaatgaggtcgggccactgcttgttcgagtgcaagacaacagactttgcgtgcttcaccgctgctgttgttcttctcattggcacgttccacaccagcgccacgtgtcactcgtccaacagcgtggaggatgacctagagctggtggcagacaccgataggctgttgctcgcagaagaagttgaaaacgactgcaaggttgccgcgcaatgccggaaagtgctccagatgttaagtattagggaggctgaaccagcggaagcagatcgggaagtcgtaatcccgtacttcggggccgtgatccgcaaacgttctcaccgcacacagccggatcctcggcctgtcactctgcctcggccgggtgggcactcaagtaaggacctcggcggccttgatattcgtccaccgcctccgtacagctgcgccgaacagacagcggccataccagccgtggaaaatccgtggagtctggaaggattctcgctagagtatataagccatcgtggatttgacagggccaatttgggcggattcctaggggcggatgaggccgcctgcacgcaggatgattcgacatcgtatatgacctcggcggcaatggagtgggatccaggttggagcgttcttgatgatatagatgataccggcagagaccttttgacagggattgaggactggtctgcagaggacttcgagatgtag

SEQ ID NO: 30 - protein, 694 aa, Trichoderma reesei
MEEPYLLPTGNPLPHRVRKMRKGTRSCTECRRRKTRCVFSPGDTVCVLCKSRGSRCIEQGYEDASLPNPRSGAARKSPTAEKASQSRNEPSVDQPPKFCGDAETSDALASERANHAPIVSLLVDVKLSASAKGGQDEPSNPKPMPWSMQPLGSKSAYVCSTIRSMLPSYDTIISVLTRNGSWWDSFRSKAYAISEAPSQTIEAFAKRTYTSSNPADIGALAIAFARSLNKHRYLYTLVDDLVISDINFLTTIEGLECLILLAKSYTDCGQPKRAWLVWRKGASATQLMGLCCGSNYSHTEMRLWCSVYHGDRFCSMLLGLPYVLNDNHYQSVINAPGMAPGFYFVLRCAIICGKIIDRNMTVGKPSFARAMELDEEMESVASSQPQDWWAVTETLDQPLEHLELNELREQLLQQFYFYWVKLFLHLPFLVETTTSSPHYFSRMACIEASKQILKRYRLLRTRMRSGHCLFECKTTDFACFTAAVVLLIGTFHTSATCHSSNSVEDDLELVADTDRLLLAEEVENDCKVAAQCRKVLQMLSIREAEPAEADREVVIPYFGAVIRKRSHRTQPDPRPVTLPRPGGHSSKDLGGLDIRPPPPYSCAEQTAAIPAVENPWSLEGFSLEYISHRGFDRANLGGFLGADEAACTQDDSTSYMTSAAMEWDPGWSVLDDIDDTGRDLLTGIEDWSAEDFEM

SEQ ID NO: 31 - DNA, 2861 bp, Trichoderma reesei
atggagggtgcagaccgtggcaaaggtacctggatgtacccgccagcgaccgcgacgccataccttccccagccggtgcaggtgcgtcgatgcttccttctgtccagcaacagcaacagcagcagcacgactccaagcttcattcttccacggcttgactttttgcatctctgcagcttcatccacctccattgatcgtagatcgtcagattcccatcacatcaaatcaatgtcactgactcacacttccagctttcacccctgactctccgtccttcgctgcttccgtctctagacttgcgcctctcaccgagtcacccccagtttttccgctcgcagcttgaagcctccacctcaacctcgagtctctctgctcctgcttacttgctcgccgcgcattgcgacagctccgagactcaatcgcctcccgcgctctccttttcgccgtcgaccgcgagctcgatcgcgacgccgttgcccggctcagtcgcagcaatggacctcgacccgcctcccgacgccgacatgccgtcattcgacctcgccggatcgtcgctcccggaagcccagccgagcctcgccaaccccgtcgagggctccggcctgtcgctcgaccgccggcccaagaagtcgtcgaccacctgcgccgtctgccgcttccgaaaggtgcgctgcaacggccagcgcccgtcctgcggcaattgccagcgcctcggcttcccctgttcctacgacgatgccgacgtcgacacatggtccatgtcattgccgcgccgccgtgtcaagcaggcctgcttgagctgccacagccgcaaggcgcgatgctccggccatctgccctcgtgcgagcgttgtcgagcccagggcatcgagtgcgtctaccggcccaacaagcgagccaagccgtcgtcggcgggagccggcattgcgggctcaaagagccccaacagcccggatcaagagtcggatcgcgacggcggcagggaggcacggggccatggagagagccgcgaccacgatgaagggcgcaatgatagcccggcattgaccgaccgtgccagctccgccagtccaggggcagacaaccaagggtaagtgactcggacaacaactgttttcgtgataggtacactgacggccttggaactggcagtccgcagatagatgaaagcttcagctccatcgtcagccgcgcattcgacctcttcttccgtcacgtccaccacatgcccatgttcaccttcctgcaccgcgcttctctaatggagcaatatcacgccggcaaagtcgacagggcgcttcttctggcactggtcggcatcacgtcgtgcttgaccaacatgggcccgggcatgcgcaaatatggaaaccgctgcatcaacgatgccgaggccctcctccttgccgactacagccggccctcgattgtcaagatccaggccctcgtcttcatcatcaaacaccgcatcctctgcaataaattctccagcgcctttgtcctgcacagcttcgcctctcggtacgcctctgctttgcgcctcaactacgaggcgccccatctccgcttcctggctcaagagtcccgtcggcgccttatgtgggccctgtactgcatcgacacgagcatctgtggcgggtatccggactttgtcctctggagagccgaccagatccacgtctatttgccgtgcaacgagcgcaacttcgagtttgacctgccgcagcagacggagaagcttgtgccagactcacaccaaccccggccgccgctggctgaggatatcggaactctggcccttcatgcgcgcatcctgcacattcgccagaagatcatcgagttcacaaaggtagctcagtatgaccgcggcatggaagccgccgagctgcagggccgtatcttcgcgctcgacaaggagctcaatgactttgccaccaacctgccgacctcgttccagttctccgaaaactcgctccgtctccgtgcctactcgccgagactatgcatctttgtcatgatccacgtctggtggcgccagtgctactgcgacctctatcgccttgccttggtcagcatcagtggaggtctgccgcagtccatgctcgacatgttcgaccagagcttcctcgagcactgtcagaggcagtgtgtcgaccactctctggcgctgactctcatcttttcactcatccagaagctgggtgcaaagccagtggcagacattgatctggccatgtgcgcgtatcagtgcgcgcggatgctcatgtaccttttccagttcggcatcttcgatcggttcggcgtcacggcagaaacggtcatggagcaagctcagctatgtcttcagaccatcaaggactgttgcgtggggcccgcggtggactgtatcgtggccgatttggagaggctcatcaaccaggaccccaaagccgcaatacgagaagggctaccgggggggccgccgaatctccaaatgacagacgggccagatgttctcagcgctcccgctgcgaccggcctgcagccttttagctccccttcaggcgctgtcaacaacaccgtctcgccttttactcatcccgtcatgactgctccctggatgtcggaaacgggctttgcctcaggactggaccagcaaagtctcccgccagaggtccatgccaatcctagcaagggcggaactcccagtctgtctgcaccgcggtcggagtttgggcagtctgatgtgaacgcatacgacagcacctttgcagggctcgggctggacgatggatttgattatgccatgggactagacatgaacatgtgggcgaccaacgggggcagctggactgcgcctggctatgggaatacctggatgtga

SEQ ID NO: 32 - protein, 884 aa, Trichoderma reesei
MEGADRGKGTWMYPPATATPYLPQPVQLSPLTLRPSLLPSLDLRLSPSHPQFFRSQLEASTSTSSLSAPAYLLAAHCDSSETQSPPALSFSPSTASSIATPLPGSVAAMDLDPPPDADMPSFDLAGSSLPEAQPSLANPVEGSGLSLDRRPKKSSTTCAVCRFRKVRCNGQRPSCGNCQRLGFPCSYDDADVDTWSMSLPRRRVKQACLSCHSRKARCSGHLPSCERCRAQGIECVYRPNKRAKPSSAGAGIAGSKSPNSPDQESDRDGGREARGHGESRDHDEGRNDSPALTDRASSASPGADNQGYTDGLGTGSPQIDESFSSIVSRAFDLFFRHVHHMPMFTFLHRASLMEQYHAGKVDRALLLALVGITSCLTNMGPGMRKYGNRCINDAEALLLADYSRPSIVKIQALVFIIKHRILCNKFSSAFVLHSFASRYASALRLNYEAPHLRFLAQESRRRLMWALYCIDTSICGGYPDFVLWRADQIHVYLPCNERNFEFDLPQQTEKLVPDSHQPRPPLAEDIGTLALHARILHIRQKIIEFTKVAQYDRGMEAAELQGRIFALDKELNDFATNLPTSFQFSENSLRLRAYSPRLCIFVMIHVWWRQCYCDLYRLALVSISGGLPQSMLDMFDQSFLEHCQRQCVDHSLALTLIFSLIQKLGAKPVADIDLAMCAYQCARMLMYLFQFGIFDRFGVTAETVMEQAQLCLQTIKDCCVGPAVDCIVADLERLINQDPKAAIREGLPGGPPNLQMTDGPDVLSAPAATGLQPFSSPSGAVNNTVSPFTHPVMTAPWMSETGFASGLDQQSLPPEVHANPSKGGTPSLSAPRSEFGQSDVNAYDSTFAGLGLDDGFDYAMGLDMNMWATNGGSWTAPGYGNTWM

SEQ ID NO: 33 - DNA, 2248 bp, Trichoderma reesei
atgaagacatcatcgagtaccccacgcatccgcaagaagaggattccggtaagcatatgccgtctgagcagcccaggggccgctgtcatgtcacgttgacctcggtagctcggcatgctccgttgctgatcgtgttggtggacagaaatcctgcaccgcatgtcgccgctccaaggtgagaggctgaactcgaagcctatagttcgtgagctcaaagttctgactgacagctaatcccaggctaaatgcgatggtaagagaccatgctcacggtaggtcttctatgtgggctgaatcggctgtgcctcgatcccagattaacggttacatccagatgcagcgacctgaagaagatctgcacttttattgatcctcccaaacaggctcatgagatgtatgcttcttgcgttaccgattccaatttgtcgatcgcacacgcaactgaccacgctcgcctcactcatgcagccggatagaggagcttgagcaacaggttgcggcgctcaaagataggcttcgagcggatgcattggcacaaagcactcaagcagagaccggcttccagcacaccagccagccaggcattggcgacaccccaatctcatactcacctcgggccacttttgtcagccccgatagagtcgcctcagcattctgcatacgttcatcacacacccaatcaccgaccaatacgacacttgccagaaaacacaccagatctcactttgagattgggtctgtgacattaccagattgcctggatgctggcctgctaagtctcgagcaagctcgtcactacttctccatcttcttccaaggatgtgaccactttgttccggtgctcgatcctcgctatgactcattcgaaagcattcgcgcaagaagcagtctcctgtttggggcaatatgtgcagttggctgccgcgttgtgacggggtctgaaacccagcaatggcatatgctcgatttccatgtcaagaggatgctgacttgcgccctggccagtccgtccatggcctcactcgagactattcaggcactgttggtccgctcgtgctatgcatcagagaggtctctccttgtcgcagctgcaacgcgcatggctatcgatttggactttgcaagttcctacgatgagatggtcaatcggtctgtggccccggcagcacggggagtatcctcggcaagtctggatcaagatacaatcaccttgatgcgaagagtgaggacatggctccatctgtttgtgttggggcaaatattgcatgtcgatgctgctgacctggcgaccttcaagtttgttggggatttacggagatgccgcattatactgaagagcccggccgcaactgagctcgatctgtccttgttctcccaagtcgagctcaatgctgtccgtggcaggatctatgactcgttgtctggcctcgtgggattcagcgatgaagacgcaatgatggacgtcgttcgcgaggcaaagatcgacattgctctatggtatgatgattgggagcacatcttcgagaagcaaagcgctcaagcgccatggcttggtgtcaacttgcgcgcacaaaaatgttggtcagagaacatggccttttgccgagtcctgcgggcgtctggtgtcgagaatgtggacttcatgtcgcccgctcaaaagtcagttgtcgccatggcaaaagatgccttggaggagcacctggatatcatgatcagagagccgaggctctatttacgcaatctgcgcttcgcaatggactttgtctgggcaaagtgcgcattttgctacttactgctgctcaaactttccattctgttgccggaaagtaaggggcgcagcagtcgagagcttgtagaacacggcaatatcctcctggccgagctaagtgaagccagcggagggaatcatagcggaagccggagcagcactggaaaacagtatcttcagcttctacaggtggggatcgggaagttcagctgtgccacccaggaagcccacgaagtacttgcgagcacggcaaatgacgacagttttgcaactgctcgacggacgcctacattggcaacacagaatcgaatggagctggactcgtttgttccggaacaattcgtctttgagtgggatttccctgggctgacgttgttttcctcatcggcgaccagggtttcgtggcttgatgatatccttgcagaggcactcaacgggagtgaggatgcatttgtttggttaccaacagacaatgacaac

SEQ ID NO: 34 - protein, 650 aa, Trichoderma reesei
MKTSSSTPRIRKKRIPKSCTACRRSKAKCDGKRPCSRCSDLKKICTFIDPPKQAHEIRIEELEQQVAALKDRLRADALAQSTQAETGFQHTSQPGIGDTPISYSPRATFVSPDRVASAFCIRSSHTQSPTNTTLARKHTRSHFEIGSVTLPDCLDAGLLSLEQARHYFSIFFQGCDHFVPVLDPRYDSFESIRARSSLLFGAICAVGCRVVTGSETQQWHMLDFHVKRMLTCALASPSMASLETIQALLVRSCYASERSLLVAAATRMAIDLDFASSYDEMVNRSVAPAARGVSSASLDQDTITLMRRVRTWLHLFVLGQILHVDAADLATFKFVGDLRRCRIILKSPAATELDLSLFSQVELNAVRGRIYDSLSGLVGFSDEDAMMDVVREAKIDIALWYDDWEHIFEKQSAQAPWLGVNLRAQKCWSENMAFCRVLRASGVENVDFMSPAQKSVVAMAKDALEEHLDIMIREPRLYLRNLRFAMDFVWAKCAFCYLLLLKLSILLPESKGRSSRELVEHGNILLAELSEASGGNHSGSRSSTGKQYLQLLQVGIGKFSCATQEAHEVLASTANDDSFATARRTPTLATQNRMELDSFVPEQFVFEWDFPGLTLFSSSATRVSWLDDILAEALNGSEDAFVWLPTDNDN

SEQ ID NO: 35 - DNA, 820 bp, Trichoderma reesei
atgtttaacacattcaagatcgaccccgagacgaacagcgtccaggagctcaggcgggcctccgatcccatctcagcccggtcgagccagcaccaggcgtgcaacaactgccacgctaaaaaggtataaagtcgcagcaagcgcgccgcttccagtcaagtgatcaactctgttgttgttcctgcgctaacagtcactctcccccgattagctcaagtgcagtggtgacaagagtggctgtgagcgctgcatcgccagccagctgcgatgcgaatacactcgctctccctctcgcagaggcggcaggaagaccagcggcagaagctccactgacagccgtggcggattggagccggccggtggtgacagcagccccggcagccagtctggcaagtcgaggcacagaagcagcaggcacttgcatgccgggactagcagcagcagcagagcccgggcctcagcgtccagggaggaagaatatggcgatgcgttggacttgttcgaccccacgacgctcggtcctgacgatggcttcgatctacgatccttatccttggaggcttcggacagtggttacggcaacagtgcctattacaaccagcagcagcagcagcagcagcaacaacagcatggtgcaggaagctggcaacacgtggcctccagcgacccgtacggcaacttgtctggcagctcatacatgggcacgaccagctccagtggccaagactacgacgtggattactacggaaactatgacgagtacggacagtatcatggccagcaaaacgatcctagatactggggcgggcagcaacaatag

SEQ ID NO: 36 - protein, 243 aa, Trichoderma reesei
MFNTFKIDPETNSVQELRRASDPISARSSQHQACNNCHAKKLKCSGDKSGCERCIASQLRCEYTRSPSRRGGRKTSGRSSTDSRGGLEPAGGDSSPGSQSGKSRHRSSRHLHAGTSSSSRARASASREEEYGDALDLFDPTTLGPDDGFDLRSLSLEASDSGYGNSAYYNQQQQQQQQQQHGAGSWQHVASSDPYGNLSGSSYMGTTSSSGQDYDVDYYGNYDEYGQYHGQQNDPRYWGGQQQ

SEQ ID NO: 37 - DNA, 2317 bp, Trichoderma reesei
atggctgctcccggttccaggccggcgatcatggcctccgtcgaaggctcatatcagaccccagccgcaactccaaccggatcgcccgatcccgacggcatggagccgttgctgcccccgacttcgatggctccggcgattggcggcgatggtggcggcgaggcggccaggccggccaagcgcggacgacgaagcaaccccaaggtcaagacgggatgcttgaactgcaagtgaggacgctttggaataccctgattatcatgcccttccgcttccccatgaccactcactctcccatgcatgtcctcttcctcctcctctcccttcctcttctctcgtattgacggaaacacataaagccaccccgggcagcccactgacgagctccatccctaggcaacggcgcatcaagtgcgatgaaaagcgtccgtcttgttcgcaatgcatccgcagcaagaaggagtgcagtggctatccggcgcccactcgcgggccccggactgccgtcgatgtccgcattgctccaaagcccttggctccctcctcgacggggctcccgcaactgcagcctactcccagcagcatagcgagcgcctccacttcaaccactgccagcctcttgctcactggccatacgattatgctgcctcctcgacgggcctatcgccgcaaacgccagaccaagcctgccgcctccaatgccgcgatgccctttatgtatgagccgtctcacaacttggccctcatgcataccgagagtttgtatttcgacctgttccgcgtccagacggcttccgagctgtccgggtactttgactcgaccttttggacatcacgggttctgcaggaatgccacttcgaaccggccattcgacatgccgtcgtcgctcttggtgccctgtacaagactctggaacagtcctgcgaacccgacctgccgccgctcgctggtgccatgagccgcctggactcggtcatgtgtcactggcaggttgccatcagaaagtactcggaagcctgcaaggccatgctgcatctcagcggcgacaagctggcaaccaacaagacccggctgatggccagcgtcctattagcctgcttcgactccttcattggcgatcaccgccaagccattgtccagatacagaccggattgaggcttctggctcggattcagtatgatcggacacaggccccttattccaatgaacgggttgaggaggatctgctgattatcttcacacgactagccattcaagccaagtcgtacgacatggccttccacttcccccatccctacgtgatccatctaggccctcaaagcctacatgatccgtcatcccccctctcagactcgggttcacctcagccatccagccccatcccgtataagttctcatccttacgagaagctcgcctggcttcggatcagctttgtgaaatgctgctgcgattcatcgaacacttgcagcgcgccaagaaggaaccttcttacactttacccccatcctggaggcagctcggagccacattccagagtcaaatcgactcatggtcacaggccttcgagcccatcttccaatcaaggctcacgcagcccggcatgagcctccttgaaaagtcgggcatcgcagcgctcaagatgttccaggtcaacaccaacgtcatcttcctcaccatcttctgcgacgccgaggtccagttcgacgctttcctctcccacttcaaggccattgtcagcctgggctgggaagtcgtcggcgacgacgagaagcgagcggccaccgagcgatgccccgatccgcggcggtgcaagcagcagcacggacacgcaaggaccggcggggaagaaggaggaccgagggcgggaagagacgcattcccaacccacaacatcaagcccagcttctccgcagacctgggcatcgtgccgccgctcttcgtcgtggccacaaagtgccgcgagccgaccgtccggcgggaggcgatccagctgctgaggagcagtgcccggcgcgagggcatgtgggacagcgagctggcggccaacattgcccaatgggtcatggagattgaggagtccgagaacccctttccggagatgcagcagcagcagcagccttcgcacggtcatggtggcgcagccgcacaggccgtcgcgctgcccagccgggccattccggaggagaagagggtcatggtcaagtcggtcgactttgatttgcgagcgaggtttgccgacgtgaccgtgggctcgagggactttcgccagggggtgcaggatcgacgacatagggcgacgcgaatcagttggtga

SEQ ID NO: 38 - protein, 716 aa, Trichoderma reesei
MAAPGSRPAIMASVEGSYQTPAATPTGSPDPDGMEPLLPPTSMAPAIGGDGGGEAARPAKRGRRSNPKVKTGCLNCKQRRIKCDEKRPSCSQCIRSKKECSGYPAPTRGPRTAVDVRIAPKPLAPSSTGLPQLQPTPSSIASASTSTTASLLLTGHTIMLPPRRAYRRKRQTKPAASNAAMPFMYEPSHNLALMHTESLYFDLFRVQTASELSGYFDSTFWTSRVLQECHFEPAIRHAVVALGALYKTLEQSCEPDLPPLAGAMSRLDSVMCHWQVAIRKYSEACKAMLHLSGDKLATNKTRLMASVLLACFDSFIGDHRQAIVQIQTGLRLLARIQYDRTQAPYSNERVEEDLLIIFTRLAIQAKSYDMAFHFPHPYVIHLGPQSLHDPSSPLSDSGSPQPSSPIPYKFSSLREARLASDQLCEMLLRFIEHLQRAKKEPSYTLPPSWRQLGATFQSQIDSWSQAFEPIFQSRLTQPGMSLLEKSGIAALKMFQVNTNVIFLTIFCDAEVQFDAFLSHFKAIVSLGWEVVGDDEKRAATERCPDPRRCKQQHGHARTGGEEGGPRAGRDAFPTHNIKPSFSADLGIVPPLFVVATKCREPTVRREAIQLLRSSARREGMWDSELAANIAQWVMEIEESENPFPEMQQQQQPSHGHGGAAAQAVALPSRAIPEEKRVMVKSVDFDLRARFADVTVGSRDFRQGVQDRRHRATRISW

SEQ ID NO: 39 - DNA, 1887 bp, Trichoderma reesei
atgagcgccgtatctaccgagaagtcacatcctaagcaagcagctgagacaacaagaccgacttctctcaatcgtacctgtgaaggctgtcgtcagaggaagatcagatgcatcattgaaagctctcaatcaatcagtcccccgaaatgcgcccgatgttccaagttcaacctggattgtatctttctgcccccggctattcggaggaggcgcaataagaacgagactaggatcaaggaactggagcaaaagctgcagcaactccaagatgccattgcccattctccagctgaagccgttgctggcaacgcgcattcagaaggatcccatcctgacagtgtctttgagtcgttccatccggactttcagcagccgtccacttcattcgcctattcactgccgccagaaacatttcttccaactgcactctcttactctaccccagaagactcgatatcaactggtttggtaagccccgagcttgcagacgatctattcacgacatttttccagactctggcgcctatttatcccctcgtgcaagttccatcggactggacatggcagcagaccagatacgccaaacctgccctcttcagggccattctcacagctgcttcgagtgaccgtgatccggcattcttcatgatcatgttccgcaacacgggcatgtatgtgacggaagaggtttccataaagggcaacaagtcgcttgacctgattcaagcacttctcgtgctatccgcgtggtactgtcccatggaggactttcgaaagctgaagtttagccattacgcaaacttggctgggagcatggccttggacttgaggtcctcaaacgacgagcagtattggattccaccagtcaaggactcgtttgccagctcagagcagttggtagaaacatgcagaacgttcttggcaagttactttctttgctctagtatggcattttcttatcgacggccgagtatcctccgctatggaccttgggttgatgattgtatacgagtacttgaagccgcaccaactgcatgcctaaacgaccgccgactgatagagtggacaaagctgcagatcattgcggaagaatgcatgtcggcggctgggctcgacaatgagtccaacgtctgtttagcggacgacagagttggtcgcgtattgaagcatggcattgaaagggcgatatcgtggaagcaccaggtttcccctgagatcatacataaaccgatggtgatgcactaccatatggtgttgataagccttaatgagccggccttttacgatgcacatgatatgcaggatttccgaccgccatatcgactgcgtcctctgcctctggccaaaagctccgatgatcctggctcaattgccatcgccaactcgcttgctcagtgcgtcagctccgcacaaacggtcatcaacacgtttctgggcatcccaacggaaaccctcagagtcatgcccgtcatcgtatacacccgcatcacatacgcagcagtcacgctgatcaagtttgatgtctccgcccgaatgctgcagagcgttgcgtgtatgttggatgacatccacctaagcccaaaggtgcttttgttacagcttcttgacaaactaatcgaagtggcggggagcgagaacgttgttgtgccggttgtctttcgaggggctttggcacggatgatgaggtggtatgttgatcaacttgagagctttcaggagccggatcaagatgaggtcctcgaaccgatgatgtacgttggcgttgatgcagaaagcagtagcgggttggatgtcggttatggtggtgctgaatcgcaaggatctgttgcggatccaaatgccagcttcttggatcctttgagaacccatcatgtagattatctatag

SEQ ID NO: 40 - protein, 605 aa, Trichoderma reesei
MSAVSTEKSHPKQAAETTRPTSLNRTCEGCRQRKIRCIIESSQSISPPKCARCSKFNLDCIFLPPAIRRRRNKNETRIKELEQKLQQLQDAIAHSPAEAVAGNAHSEGSHPDSVFESFHPDFQQPSTSFAYSLPPETFLPTALSYSTPEDSISTGLVSPELADDLFTTFFQTLAPIYPLVQVPSDWTWQQTRYAKPALFRAILTAASSDRDPAFFMIMFRNTGMYVTEEVSIKGNKSLDLIQALLVLSAWYCPMEDFRKLKFSHYANLAGSMALDLRSSNDEQYWIPPVKDSFASSEQLVETCRTFLASYFLCSSMAFSYRRPSILRYGPWVDDCIRVLEAAPTACLNDRRLIEWTKLQIIAEECMSAAGLDNESNVCLADDRVGRVLKHGIERAISWKHQVSPEIIHKPMVMHYHMVLISLNEPAFYDAHDMQDFRPPYRLRPLPLAKSSDDPGSIAIANSLAQCVSSAQTVINTFLGIPTETLRVMPVIVYTRITYAAVTLIKFDVSARMLQSVALAGSENVVVPVVFRGALARMMRWYVDQLESFQEPDQDEVLEPMMYVGVDAESSSGLDVGYGGAESQGSVADPNASFLDPLRTHHVDYL

SEQ ID NO: 41 - DNA, 2066 bp, Trichoderma reesei
atgccccatatgcgcgaggtcaagacgtgtgaccgctgtcgccatttcaagcggcgctgcgacctcctcaagccgtcgtgctcgcgatgcatccaagcgggagtgcgctgtagcttcgatgtcaacggggtcgccgtgcccggagccgctgccaatggcggtgcgtcttcctcgcccaacgcatcgcgacaggcgagggccggcgccttgacatcttcgtctcctgccaccaatggcgaccctgcgctggctacaccgggtcggaacgggctcatctcgcccacggcatccaccgagagccccgagcccgccgtggccctcgatgccaatggcatgccgatcgagggcgcctctgcgtccgcgtcggccacgaaccagctgcgcgttgtccgcaagcgcaagcgcaactgcctgagctgccttcgctgccatcgcctcaaggtcaagtgtgacaaggaactgccgtgtggccgctgcaagtcgagcggcaacggcagggagtgctattatagctacaacaaggggcccaacggtggaaagttcccttgtcccaccgccccgtcgagcaccgaggagacgaagacggtgcttgctacctggcaggtccaacacaaggtgcgaggctccagccattggcgggacctcatgaccaaggtatgcagagagcactttgcatggaagacttcctcgagatgtgctgactggtgggatagattggcacattgacaacgctagactccgcgcccctggctgccgctctcgaagacgtagccaccaatgcttgcctggccaactttacgctgccgggcaacttcccctttggcacccctggtgcgaccaagtactatactcgcgatgccgtcactcgactgttggccagcgagagggcaaactgcgatcggtacctccagcgatacctggatctgctcgacgtggtcaaccccatcgtggacgtacacatcttcacgcgcgaagttgagcgatactggatagaccccaacgcctcggacctgtgctggctggcgcagttcctcatggtcatgggcctcggcagctttgcctctcccgagggggagcctcccgtcgccacggagctcatgatggccgccgaggcgtgtctgatgcagacgcccttttccttccggccgactctgttggcgctcaagatgctggccctcatggttgtcgccaagcaggtctgcaaccccacctgctggtcggtcgactcgtgctggtccctgctcggaatgctcgtgcgcacggccttcatctacggactgccccaggacccgtcgagcccagaggacgcggggcgcaaccaggacgaaaaggacgcccgacggaagctgtggctcacgatcctgtacctcgacatcaaggtggccatgtgtaccggaatgccgcccctcacacggccggacgagctcggcaccctcgagaagattcccgaatggggtccgccggacagtctccagatggtgctgtaccagtcgctgccgaccgtgctggccgtcatggcgcagatcaattccaacaaggagcagatctcgtaccccgacgtgctgcgatacaatgcccagctgcgggagctcatgagccacgcccagcgggtctgtaccggacagctgcagcgtgtcacggtcgacatcttcttgcggcggtgcctcatggtgctgcatcgtccctttgccctgcacccagagggccccgtcatgttccccgagtcgtactggtcgtccctggagtgctgtcttgcgctgctggtgcactaccgcgagatgtggtgcagcgacccggatctgcgcctggacctggtcggacgggcctttgtcctcgacttcttctccgccaccttgacgacatatatccacgtcctccgcgctgacgcccctctcacgggcgctgctgccatgggatgcgccattccgccgcgacagattatcctcgacacgttgcgaagctgcgtagagatttggagctcggaaaaggacaagagcgtgtgctaccgcaccgggtacaacctgcttgtcgcggtgctgaacatggttcccaagaacaagatttga

SEQ ID NO: 42 - protein, 658 aa, Trichoderma reesei
MPHMREVKTCDRCRHFKRRCDLLKPSCSRCIQAGVRCSFDVNGVAVPGAAANGGASSSPNASRQARAGALTSSSPATNGDPALATPGRNGLISPTASTESPEPAVALDANGMPIEGASASASATNQLRVVRKRKRNCLSCLRCHRLKVKCDKELPCGRCKSSGNGRECYYSYNKGPNGGKFPCPTAPSSTEETKTVLATWQVQHKVRGSSHWRDLMTKPLAAALEDVATNACLANFTLPGNFPFGTPGATKYYTRDAVTRLLASERANCDRYLQRYLDLLDVVNPIVDVHIFTREVERYWIDPNASDLCWLAQFLMVMGLGSFASPEGEPPVATELMMAAEACLMQTPFSFRPTLLALKMLALMVVAKQVCNPTCWSVDSCWSLLGMLVRTAFIYGLPQDPSSPEDAGRNQDEKDARRKLWLTILYLDIKVAMCTGMPPLTRPDELGTLEKIPEWGPPDSLQMVLYQSLPTVLAVMAQINSNKEQISYPDVLRYNAQLRELMSHAQRVCTGQLQRVTVDIFLRRCLMVLHRPFALHPEGPVMFPESYWSSLECCLALLVHYREMWCSDPDLRLDLVGRAFVLDFFSATLTTYIHVLRADAPLTGAAAMGCAIPPRQIILDTLRSCVEIWSSEKDKSVCYRTGYNLLVAVLNMVPKNKI

SEQ ID NO: 43 - DNA, 2605 bp, Trichoderma reesei
atgaccctcgactacaatgtcaacggcgggggcgatgccttactcgatctgacctacgaacaacaccagcgggaccgggctgctgcgtacagctactcgacatcaaccactgctcactcgagtcctggccctgcgtctaacctcgtcaccggctcgtggaatctgcccatcgacatgaacgccaacgccggctcgtctcagggagctcgaggcagctccccggaggaataccacccaaactcgcctctctcacacatgtccagccctcacagccaccactcaccggctcagcactcgccctaccagcaacccacaaacccgttgaccgactgggctcttcaccaccaccagcaacaggctgcttccgcggcgcagcagcaccaggttttgcccgcccacgacctgacgcagtacatccaggactccctgttgaacttcaacaacaccttttctggataccatcatgtcgactatctaccggccacgacgcaggctgcgatgcagacgcatttgctcgagtcgcccttcagctcgatgcctgctctggatgataccgcacacgcaatgcactggggaggcgcgggcatgggaaattggcaggactttcaaaacaccctgcagctggacgggctgcagaccgttggcagcaactcgcccactggaacctacctcgaggtgctctcattaccgtcttcgtcctctggcgatggctgggccatggtggactgggggcatcccggccacgagctcttccagcctacgcccagtgccgccatcttcaacccctcccagacactgcacttgaggaccaactcggattcctccgacggccgcaactcggtcgaaatcggtagctttgaggaagttgccccctttccctattcgcccttctcaccagactctgatggccaagccgagaaccacaacaacggccaccggaactgctactcggccgatggccatcatcaccaccatgatcacagccatagccatggtcatacccatggccacagccatagtcacagtcacagtcacagccacgtccacagtcccatcgcggtgactgccccagtctccctcaagggcgagtcgtctgcccgtcacagccctggcagcggagcgggctcggtatccccctcttcgtccagcaccacctcaaggaggagtgccggaagtgctggcattcgcaagagcccgattgccaaggcacccaaaaccgtcgtcaggcgcgtctccaatggaaagaaggatggatctgtcgagaagaaggtgggccgtagaaagggccctcttttgccagagcagcgaaagcaagccagcgagataagaaagctgcgtgcttgcctgcgatgcaaattccttaagaagacttgcgataagggagagccctgcgctggatgccagccgtctcacgcccgcctctggcaggtcccctgcacgcggatcgacatcaaggacattggctacttcatgaaggactggaaggccgactatgagaggcacatggaccgtggagtgtccgtctacaatgtcaagggcttcgcgcagaaggagacgctcatgtggatcacccacggctacggctttgccctgcccgtcatggtccgcgaggtctttgttgccgatgacagctgcttctccgtcgagtgggtcgagtcgtacgttgcctccaacgacaccatcgactttgagactcggactgagaagctcgatgtcggtgccgagggcattcgcatcgacgctctctccgagtacctcgacaagcacatcgagggtccgtttgaggacttcatcgacgaccacttcgagggcactcccttcatcaccgagatcctcaagactgcccaccgctactacgtcaaggagaagatgcccgtcatccgaaaggccctgaagctggtccttgcctacaacctgaccatgcacatcaccatggtcgagagccagggatccgagatgccccttgacggccagatcgacgacgaggactccaagtactacggccgcaccgttgcgcctgtcatgatcaacttccagattaagtgtgctctggctgacatgtggcgcgagcttcagaaggacatcctggaggagctctctgccctctactcgggtgtctacagcggcgagaagatgaagaactggccgaccatcttccttctggcgtccatcctgctcgccgtgtgggaggagatgcagtttgactgccactaccgcgtgccagaccccgtggcggtgaacaagttctgcaacgacatggaaacgacccctgtcggtgttattgtgggcctcttccacgccatctctcagaagctcccggccttcaccgactgggacacgcgtgagcacggccacctgcttcacaacaatgttgccgtctgtgatgccatgactgaggtgcgacagcatgttctcaagcatggtaagtttgcaccttttcgtgtcccgtcggacatgacccgaaaagttgacgaaagtgtatgctaacaaactatgttttacagaggcttacctgcgaacccggagagaagccaagtttgaccgctacgattttgattcgctttcgaacaagttcttgtcgaagcttgttatccgggccaactaa

SEQ ID NO: 44 - protein, 840 aa, Trichoderma reesei
MTLDYNVNGGGDALLDLTYEQHQRDRAAAYSYSTSTTAHSSPGPASNLVTGSWNLPIDMNANAGSSQGARGSSPEEYHPNSPLSHMSSPHSHHSPAQHSPYQQPTNPLTDWALHHHQQQAASAAQQHQVLPAHDLTQYIQDSLLNFNNTFSGYHHVDYLPATTQAAMQTHLLESPFSSMPALDDTAHAMHWGGAGMGNWQDFQNTLQLDGLQTVGSNSPTGTYLEVLSLPSSSSGDGWAMVDWGHPGHELFQPTPSAAIFNPSQTLHLRTNSDSSDGRNSVEIGSFEEVAPFPYSPFSPDSDGQAENHNNGHRNCYSADGHHHHHDHSHSHGHTHGHSHSHSHSHSHVHSPIAVTAPVSLKGESSARHSPGSGAGSVSPSSSSTTSRRSAGSAGIRKSPIAKAPKTVVRRVSNGKKDGSVEKKVGRRKGPLLPEQRKQASEIRKLRACLRCKFLKKTCDKGEPCAGCQPSHARLWQVPCTRIDIKDIGYFMKDWKADYERHMDRGVSVYNVKGFAQKETLMWITHGYGFALPVMVREVFVADDSCFSVEWVESYVASNDTIDFETRTEKLDVGAEGIRIDALSEYLDKHIEGPFEDFIDDHFEGTPFITEILKTAHRYYVKEKMPVIRKALKLVLAYNLTMHITMVESQGSEMPLDGQIDDEDSKYYGRTVAPVMINFQIKCALADMWRELQKDILEELSALYSGVYSGEKMKNWPTIFLLASILLAVWEEMQFDCHYRVPDPVAVNKFCNDMETTPVGVIVGLFHAISQKLPAFTDWDTREHGHLLHNNVAVCDAMTEVRQHVLKHEAYLRTRREAKFDRYDFDSLSNKFLSKLVIRAN

SEQ ID NO: 45 - DNA, 1698 bp, Trichoderma reesei
atggatcaaaccatctccaccaagagccctcctcctcctcatcctcctcctcctcagaagagccgccgctccaagaagggctgctacacatgccgcatcaagaaagtcaaatgcgacgaagtccggccccgctgcgagcgctgcgtccgtctaagacgcctctgcgactacgagcccaggatgaagcacaacgacctcctggccatctccaggggctacataatggccctcgagagctccaagtcactgccgacgtgggcgcagcgcgcagcgttgacgagggctctggctcttcctactcggcttcctgagtttccgagcagtgccagctcgctgaatctcacctcggccgatcatgaggcgattcggtattttcggacgacgtttgcgcggcttcatcataccaagaatccggattactcgctcttttcaatcatcttcaccttggcacaggatgagccaatggtcatgcacgcgttgctggctcttggaggccacgagattgaatttcggcgcagtagcacttcagaaggatcggatggctcaactcagctggtaggacgagatggtaataaatggacaccagttcaacactattcggccgctctcggcctcttggccgatgccatcggaaatgcagacgatggtcggcaacttgagctggatcccatctgcgcagtcttgtatctgatgctggtctacgaacaaaagtacggagacggaacatgctctggtctgtcaaaccaccttgccggagcggccttgatcgtaaggcatcgctgccagcgcctatcagaacagatagccaagcgtggatggcaaggcacgccagtgctggcgaggatacagccgcaaagccagtcagaagagcctggcttgtcgctcttcgtaatcaggctcctggtctggatcgctctctgcgatgctacggccgccagctttgggctcggcggccagttcaacagcgagctgaaagacatcatggggccagaagatgcttcgtccatttcggggtttgatgtgctgcatagatactcaaactccttataccgctcaatgtggggagatgcgtatcctcaagtcgaactgctggatgacgttgagaatcgggacattttcgcctttctatgcgcatccatgcagcttcgaagcatggtggctaatctggcgaagcttggcgcagacgagctcaagctccgtttaccagctgttgaggcagcctttagacaggttgatttgcgatatggagagttgctcgaggtggcgtccgacctgtctctatcgaccgataactcacatcgtctggtggccaatatccgtgctttcatcccgatatatcacgctgcaaagctcgaactgacgcgcgtgctgcggagaaatgggacaactgcaaagatggaaattgtaccagaggctcatatagcagccattgtcgacttggccattcaggctgataagcaccagggagtcgaaggcaccattagagtcgcatggccgcttttcattgtggccctggagaccaacagcgtggttcatcaacgctggatcctcagccggttccaagcgatgagcagctatagcagaaatctggagcgtgcgcaccgctttctcaaagaacacctcgggatgccgcttgagaagggtgaagattggttgcagagattcagcctagcaaacggggagatattcgtgatatag

SEQ ID NO: 46 - protein, 565 aa, Trichoderma reesei
MDQTISTKSPPPPHPPPPQKSRRSKKGCYTCRIKKVKCDEVRPRCERCVRLRRLCDYEPRMKHNDLLAISRGYIMALESSKSLPTWAQRAALTRALALPTRLPEFPSSASSLNLTSADHEAIRYFRTTFARLHHTKNPDYSLFSIIFTLAQDEPMVMHALLALGGHEIEFRRSSTSEGSDGSTQLVGRDGNKWTPVQHYSAALGLLADAIGNADDGRQLELDPICAVLYLMLVYEQKYGDGTCSGLSNHLAGAALIVRHRCQRLSEQIAKRGWQGTPVLARIQPQSQSEEPGLSLFVIRLLVWIALCDATAASFGLGGQFNSELKDIMGPEDASSISGFDVLHRYSNSLYRSMWGDAYPQVELLDDVENRDIFAFLCASMQLRSMVANLAKLGADELKLRLPAVEAAFRQVDLRYGELLEVASDLSLSTDNSHRLVANIRAFIPIYHAAKLELTRVLRRNGTTAKMEIVPEAHIAAIVDLAIQADKHQGVEGTIRVAWPLFIVALETNSVVHQRWILSRFQAMSSYSRNLERAHRFLKEHLGMPLEKGEDWLQRFSLANGEIFVI

SEQ ID NO: 47 - DNA, 2487 bp, Trichoderma reesei
atggaacgtacgctcgccatcttgacgaacgaacaagactttacccggccagacgataacagcatgtccccggacgagggatcctacgacggcatggtgctgccttcaccttctcccaccgacaccgcgtggagacctggcgtttccgcggcgcctctccagacgggccagctctcgcccgtcagctccaacacagacgccaacatggacaacgggccgtcgaccatgcccatgatgcaccactgctcctcctcctcctcctcgtcatgcgtggactcgtggaatgcgggctacggcaacccagacgagggcgaggaggacaaccagtgggagcagtgggagcagggctcggacgaggccctcgccatcccgaagctcgagccgatcgacgacgacatcaacctggaggacgtgagcgccgcgcctctgtcacagacgccaccaaacgagctgctgggcggcgccccgatcaagcagaagaggccacgaggacggccgaggaaacatcccctgacgcccattgtcaacgccaacaaggtgaccaagggccggtccaagaccgggtgcattacgtgcaggaagcgcaagaagaagtgcgatgaggcaaagccgagatgtatgccagccattttctttatgtttatctccactggcctcctttatgctcttccctcgtgttgcgttgccacgcatgatgaacttgtgtgcattttcaaaagctaatatgtgagattctgaacctcgataggtatgaactgtgagaagaatgccgtcgtctgcgagggatatcacgagaagcaagtctggaagagtggcagggaacgggctgaagagggtatgtcggacagaaaagacatgacaagactagaagtagatggacaagggacaagagaacggcttgtggctaacccatcagcagagcgacagcggtgggaaaatctccccatcatcaccatgcagcccatcttccacggcgtggagacggccgaggacaagatcttctggaagcactacgtcaaccacttcagcaacgtgctgaccgtcgagggagaggccaagaacgccttcaaggacatcatcctgcagctcgccaaccggcaccagggcctgatgcactcgatcctcgccgccagcagcaagcacatcgactgggacacgccctacggcatcaagatcctgcagagcaacccgaccacgagcaaggaggcgctgcagcagcgctccgagtatcaccacgacgagggcatgaagcgcatgtacgccgagatgaaccaggagatggacaagagtaaccccgagtacaagacggtcctcgccgcgcggtacggccagatgatgtgcctcctgctgcagacgcgagcagagggcaatccacggggcgaccaccgggtccatctgcaggcgtacaagcacctggtgcagcactcgccgcccgaggactccaacttcctgaccttcatcaccgagttcttccagtaccacatctacgccgacgacctcttctggtaccccgagaagcggacggagcgcctcgcctccgaggactgggagccgtcggcgcccatccatcctccgcgtctgctgggcgtcgccgacggcctcttccgccacctctccgagatcaccagcatccgcaacgccatccgcgtcaacatggccggcgccgtcgaccccctggtggactacacgagcctctacaaggccgccgagatcgacgccgccatccgggagtggacgcctcgctggccctcgggcgacagccgcgaccgcgtcggcctgctgtacaagcagatgctctgggtctacctcttccgcacgatatacccgccctcggcgctccccggccgtcgcagcaccattggctcgctgcccacggccatgttctcctcgatgccgacgaatcctcacccccgacgggcctcgatggccgcggccattggcactgccacggctggctccccgctcatgggggctgtcaatcccttttcccggtcgacgacgaaaacgacgcacagctgcccgtccacgcgccaaccctcacggaccaactccatgcatgagggcgacctgcacgcaaccaccacctctgcctctgcctctgcctccgcctccgagtcacacagccaacaacggcccccctcgtctccgccacccgcccgccgacctgcccaggacgataagcgcatcacgctggccgtcgacgaatcgctgggcctcctcgagtccttcaagccgtccgatccctcgcagacgctcctcctgatcccgtgcctggtcatcggcacggcgtgcttcgagcccgaccagcggtaccgcatccgcgccgccgtccgggccgtgcgagggtacaccggcctgaagaactgcgaccgagtcctggagctgctcgaagaggtgtgggcactcatggagcagggcgactgggtggcggtatgggactggcagagggtcgcgagacgaatgggcctcgactttccctgcacttga

SEQ ID NO: 48 - protein, 756 aa, Trichoderma reesei
MERTLAILTNEQDFTRPDDNSMSPDEGSYDGMVLPSPSPTDTAWRPGVSAAPLQTGQLSPVSSNTDANMDNGPSTMPMMHHCSSSSSSSCVDSWNAGYGNPDEGEEDNQWEQWEQGSDEALAIPKLEPIDDDINLEDVSAAPLSQTPPNELLGGAPIKQKRPRGRPRKHPLTPIVNANKVTKGRSKTGCITCRKRKKKCDEAKPRCMNCEKNAVVCEGYHEKQVWKSGRERAEEERQRWENLPIITMQPIFHGVETAEDKIFWKHYVNHFSNVLTVEGEAKNAFKDIILQLANRHQGLMHSILAASSKHIDWDTPYGIKILQSNPTTSKEALQQRSEYHHDEGMKRMYAEMNQEMDKSNPEYKTVLAARYGQMMCLLLQTRAEGNPRGDHRVHLQAYKHLVQHSPPEDSNFLTFITEFFQYHIYADDLFWYPEKRTERLASEDWEPSAPIHPPRLLGVADGLFRHLSEITSIRNAIRVNMAGAVDPLVDYTSLYKAAEIDAAIREWTPRWPSGDSRDRVGLLYKQMLWVYLFRTIYPPSALPGRRSTIGSLPTAMFSSMPTNPHPRRASMAAAIGTATAGSPLMGAVNPFSRSTTKTTHSCPSTRQPSRTNSMHEGDLHATTTSASASASASESHSQQRPPSSPPPARRPAQDDKRITLAVDESLGLLESFKPSDPSQTLLLIPCLVIGTACFEPDQRYRIRAAVRAVRGYTGLKNCDRVLELLEEVWALMEQGDWVAVWDWQRVARRMGLDFPCT

SEQ ID NO: 49 - DNA, 2328 bp, Trichoderma reesei
atgtcagcagcagtaagtgatgcctcctgacagcctcgtgttacgcccctcaagctgacctcctacctacccaacagggggagcccctacggcctcgtgggcttctcatcccacgcctcattagggacccagattcctgcattacttgtcgccgcctgggaaggagatgcaagctgggcaagcccacttgtgttacctgccagaagttcaaccttgagtgcatccaaccagtcaataatctcttcgacgaaaacggcgccgagttcgccttggactcgggcgatggtaggaagaggcaaaggacgggctccatctctcaggagggggttgagagtgatgccgtgcctgccagcaagcctgagcctgaggagtctgagcctgaacctgagtctgagtctgagtctgagtccgagctggagccggactcggagttggagttggagtctgatgcggagtctgacgcggagcctgacattggctcgcagtttaacattgcgatacagatggagctttgcaatatgaccagtcacctcgagcgccagaatcaagaagacgatgatgattcagttgaccttgaccccaacgaggctgccattctcgagaatctgacacgacagctcaatcgggcccagcagactcgtctccgacagatccccggctacgttcagggccctctcggcgacgtgcctggtcctgacggcgaccaagatcacgactatgcttgctattatgactaccgctacgacatcgactctgacgatgccagcagcacaggccgtgacagaaagcgaagccacaccccttataccttggacgcgacttctcgcacctccaccgtgtaccaggtcaagtgcacttgggacgggcagggccctccccctctgtcctctcgtctcaacgatttcattgcgtacatgaaggaatgtcacttgacgcatgcgccatccgccgctctacctttgtctctgctgccgcaagacgattacactgcggccgagatgctccagtactactcgaagcgcccagaggaccgccagacgaagcctgtccctgatgtgaaccccgtcaaccccaagctcattgacctggcgtattccaactccctcgtcctgcagctcatcattgcccaaagggtcaatcaccgccaggtgtcatccgccatgttgccgaccggcgaaagggctgaaggcttctacgctgctgccattgccgagttcgggccaatgattgacagctacctggcggggaacgagcaggatatgcttccgctcactctggcaagcctggtcatttctctcaccgaggtcagtgtgtctcccaacatcctagcgtgcccatggtcttagccccccaccccctccccctccccctttctttttccctcatacgctcttcttgcaggaaccaaggactgatcggtttatacgtatcttctcacagagggcgcggcttgacaaacgtggtcaagctcacaaccatcccaccgcagccatgggtatcttgaagacactcttgacccttccccacaagcagatgtgtaaacaggtcccgcctatcttgctggagtattacatgcatgctgcttgctttgcatgtgttgcagccgatgtcaccaaggcggagtcaattccatttatgagcgaggcactccgagacgccgtcgacgacctagtccaggcgaaatatataggcaagctctgcgggaactggctgtcgatcatggttgtcgtacagagcatctttgagctgggcatgaagatgcggccgtttgccgatgacgcatcgaacgccccggcgggcggtccatccaccggctacttacccaaccactttgttacctttgggcagattcaagagcggctcacgcgcttcgtacctgacaaggaccccgactgcgaaagccctgaggcggccattctcttcaagaacgccgccatactatatctctggagtctcttggaatggccaaacgtctcgaagccccccggatcgtataccaactttatgaagatcgcttacaagatcgcgctcctgcaactgagccgcatctcggaattctccagcatcaacaaggtcctgtgctggccattgctcatcgttgggtgctttgccaaatcggcaaaggtcaagggcatcatcacgtcgcggctcctcagcatcgcgggccgcttcaaggtcggcaacgcgctcgagacccttttcctgctgcagcacgtctggggcctgccttttgagaggcgcaacccctggatggtccacaagtctatccgcgcgacgaggtgctgcggatgcgtctgtaccgactgcatgagtcgattgtttatctga

SEQ ID NO: 50 - protein, 708 aa, Trichoderma reesei
MSAAGEPLRPRGLLIPRLIRDPDSCITCRRLGRRCKLGKPTCVTCQKFNLECIQPVNNLFDENGAEFALDSGDGRKRQRTGSISQEGVESDAVPASKPEPEESEPEPESESESESELEPDSELELESDAESDAEPDIGSQFNIAIQMELCNMTSHLERQNQEDDDDSVDLDPNEAAILENLTRQLNRAQQTRLRQIPGYVQGPLGDVPGPDGDQDHDYACYYDYRYDIDSDDASSTGRDRKRSHTPYTLDATSRTSTVYQVKCTWDGQGPPPLSSRLNDFIAYMKECHLTHAPSAALPLSLLPQDDYTAAEMLQYYSKRPEDRQTKPVPDVNPVNPKLIDLAYSNSLVLQLIIAQRVNHRQVSSAMLPTGERAEGFYAAAIAEFGPMIDSYLAGNEQDMLPLTLASLVISLTERARLDKRGQAHNHPTAAMGILKTLLTLPHKQMCKQVPPILLEYYMHAACFACVAADVTKAESIPFMSEALRDAVDDLVQAKYIGKLCGNWLSIMVVVQSIFELGMKMRPFADDASNAPAGGPSTGYLPNHFVTFGQIQERLTRFVPDKDPDCESPEAAILFKNAAILYLWSLLEWPNVSKPPGSYTNFMKIAYKIALLQLSRISEFSSINKVLCWPLLIVGCFAKSAKVKGIITSRLLSIAGRFKVGNALETLFLLQHVWGLPFERRNPWMVHKSIRATRCCGCVCTDCMSRLFI

SEQ ID NO: 51 - DNA, 2436 bp, Trichoderma reesei
atgaagaagcgatacccaccgctcctcccgtctgtccctggacagccatcaggcccggcagagccttcgtcgagctcgcagcagctcaagcgacgtcgcaccggcgtttcggtcgcctgcaatgcttgtcgtcggaagaaaatccgggtaacttccacctgaaggttgcttgggagctcagtccctgacgcatggagagctctcatcctaatgtttagacgttaatgctcaatgatagtgcgatggcctgcggccaacgtgttccacatgtcgggagcttgccgtccggtgtacataccgcgacgactacaagctgaccccggaggcgcagggtctccttgtcgaggtgatgcgtctcctcaacagcctgccggagcgagaggcgattcgcatgctgcggtatctgaagagcgagaccgacgcggcagtgattctgtccacgctacgaggggggatatctgcgatccatcagccgtcggagctccgtattgccgtggcgacaatggacaattcctttcatgccctccagcttggatcacagaatccagttgcatacccatatctgcctccgcttcaaccgcaggctttgccgagggatgactttcggcggctcactacgatgggaagcaagccttcgtctccctattctccttctcgacggatgctctgggaccgcgatcccaccgagtcccaggcaccgctctgcgatgaacggctgcatcagctggatatcagtcactggaccaatgctccgatcaacaacgagtcggccgcccgtgccatctccctgtatctggagacggatcatccgctgctgggcttcttcgagcccaacctgttcgtatcggacctgatcaaccacaaacatgactactgctccccaatgctggtcaactcgctgctgtattgggcctgcgtgaataatgcccgtgccgtctggtatcgaatccaccgcagctgacttgattgaaagcaaatgtatagcgccgtcgacccaggcatcgatgctctcgcagcctacttctgcgccgaggccgagacgatatggaggacggaacgagagtctgactcgcttctcaacctggcctccgccctcttcctaggcctgggctacctcggccaaggaagagaccatgctgtgctgtcgtacacttcgcaggctaccaagatggccaccaggctgggcctctttggcgtcgacgaacacagtcgtgcgaagcccagcatcgacaagctgtccaaagaggcagccagtgcgtatatgtacgccgcatggggatcattcaactggatcaggtgggtgcctcttttccccttgtttcttgcgtagacatcgagaagggttcgtcgtgtgctcacggaactcgtccctttccaacgtgtgcagtctcatgtcgctcttctaccgccaaccgggaatattgggccctcggagccccccgagcttgcccatacccggcatggaggaggacatcgaggccgcctcgtcagccacaagccctgggtctcctcgcagggaaggacccgagcccgagcctcagtcgcggtacatgggaggcgtatttccctacctgtgccagttctggagcatcatgtatgaggtcagtctagcgtatgacgacagccaatcgtctctggatagccaagggaccttgtccttcgcggagcataagttccgccagctgctcgcttggagcaacactctcccgtcacgcctcctccgagccaaccagaacccgcattatgtccaagttcttcagtaagtttcctctgctccgtccctgtcccatctttgactcgcaatggttcgtcttcccgctctgggccggtgttgcttaacacgtgggtctctctagcatatggttccatactgccgttctctgcctcttcaggccgtgcatccaggagtttggtgttgctcgacttcgaacgatggttaggagcatcagctccccggatattgtgtatgccgcttcggtcgcgcagctgaaagacctcgtcctcaatttcagactccatttcgcctcctctacgtacaccgtgctgtggcatacagccttgatctacattactaacgagctcctcaccggccccaaggacaatgactggttcttctactttttgatatgcgtgtacggctatgaacgtctgagccggtcctggcgggtgaccacgtccatttccagagccctgttgtcaatggctctacgaaaggggggcataaccagtaccacagcccggacaatcctgaaagatcttggaccggacgactttcgcaaaaagtacggcgagatccgggccacattcatggcagacttggacatggccgaggaggatcccagcaatgccactgttgagaggcaggctgaggactttgagcacaatgccatgctgcgagactatacaaatatactagacgcggacgaggcggcatga

SEQ ID NO: 52 - protein, 688 aa, Trichoderma reesei
MKKRYPPLLPSVPGQPSGPAEPSSSSQQLKRRRTGVSVACNACRRKKIRCDGLRPTCSTCRELAVRCTYRDDYKLTPEAQGLLVEVMRLLNSLPEREAIRMLRYLKSETDAAVILSTLRGGISAIHQPSELRIAVATMDNSFHALQLGSQNPVAYPYLPPLQPQALPRDDFRRLTTMGSKPSSPYSPSRRMLWDRDPTESQAPLCDERLHQLDISHWTNAPINNESAARAISLYLETDHPLLGFFEPNLFVSDLINHKHDYCSPMLVNSLLYWACKQMYSAVDPGIDALAAYFCAEAETIWRTERESDSLLNLASALFLGLGYLGQGRDHAVLSYTSQATKMATRLGLFGVDEHSRAKPSIDKLSKEAASAYMYAAWGSFNWISLMSLFYRQPGILGPRSPPSLPIPGMEEDIEAASSATSPGSPRREGPEPEPQSRYMGGVFPYLCQFWSIMYEVSLAYDDSQSSLDSQGTLSFAEHKFRQLLAWSNTLPSRLLRANQNPHYVQVLQPCIQEFGVARLRTMVRSISSPDIVYAASVAQLKDLVLNFRLHFASSTYTVLWHTALIYITNELLTGPKDNDWFFYFLICVYGYERLSRSWRVTTSISRALLSMALRKGGITSTTARTILKDLGPDDFRKKYGEIRATFMADLDMAEEDPSNATVERQAEDFEHNAMLRDYTNILDADEAA

SEQ ID NO: 53 - DNA, 1614 bp, Trichoderma reesei
atggcggcgtctacgctcttcatgccctctgcgggccccctctccaagaggctgtttaaccatgtcaagatgcttgctcacaaggtcacgaatgacaagtacaaatcggtagaaattgtcttggcgttcatgacccacatcccctggatatttccgggagaccacgcaatggacgacgagacgtgcatgtatattgcgatggcgacaaccattgcgtttgacctgtccctccacaaggtgctgatgcccatggacgtcttggagtcgtcgacgacggtgtcaaagggagagtgtctggatccccggaccgcccttgccattgatgggttcccagacgtagatccgtggtctgagcagggccagctgctgctgcgagcgcgggaacgatgttacatttcgctatttgtcgtggagagagggtgagtgccggcttctcctcccatcttaactcctcgtctgacactgttctagaatggcattggctcgcggtagaccttttatgataccaattacacgaaatatcaaggattgcgacacctggcatagatcaccgatagcagacgagcaggatggtcctgtggcgtccatggctgtcatgagacgcaacttggtacgacggcgactgcgatcctggcctcgacaatctcatgctgactcttgcaggatgggcttttcaacacagttcgagcactttgcgatggctctcaggccagcaacagtgatgggttcttggtggctcgatcgtacgtcgtcacccgagctcacttaacatcggcagaatcactaatcgatgcacaggattcaaggagctatagaaagattcttcgaccagtggctagcggaatggggcatgatgattggcttgggtcctggtaagtctcagcggaattggacttgtctaagagacgtcacctgctgactgcaatggcagaacgtcgcctcccgccatatgtcgacatcctcgtcgcccacactcgcctctcgacgtacggacgcattatcaaccatccgacggcgcctgtcgaggtccgcatcttcttccgaaccgccgggttatcggccgccctgaacgtgatgagggctgcgattcagggagagtcgcaactgcaatcgatgcccaacaacacggccattatgatttcgtttgccgcatgctttgccctgaacattagcgcgtatgccccagacggctccgggcttgcaccgagcatcaggaggttgatcgaagaggccattggcgtgctggagcgtattggcaccgtcacgccgcacaggaatgggctctcggtgctgtatggaaagcatctccgacatctgctgcggatatcgggctcgtccaagggcacccggccgcccaaggcacagcggacaacgccgtcgtcgtcggagccgatgcaggacacgccgccggcgctgccgagcagcttcatggaccagcagatgctgtggccggacatggtgcagttttcgaccatgtcggacgaccagattgcgcaggtgttgaatcagccgggcaacgtgtttgagccgtcgtcgtcgtttgggggcaacatgtcgtgggaggagatgaacaactttgactggctgaactggccagctttcaatgcaaattag

SEQ ID NO: 54 - protein, 464 aa, Trichoderma reesei
MAASTLFMPSAGPLSKRLFNHVKMLAHKVTNDKYKSVEIVLAFMTHIPWIFPGDHAMDDETCMYIAMATTIAFDLSLHKVLMPMDVLESSTTVSKGECLDPRTALAIDGFPDVDPWSEQGQLLLRARERCYISLFVVERGMALARGRPFMIPITRNIKDCDTWHRSPIADEQDGPVASMAVMRRNLDGLFNTVRALCDGSQASNSDGFLVARSIQGAIERFFDQWLAEWGMMIGLGPERRLPPYVDILVAHTRLSTYGRIINHPTAPVEVRIFFRTAGLSAALNVMRAAIQGESQLQSMPNNTAIMISFAACFALNISAYAPDGSGLAPSIRRLIEEAIGVLERIGTVTPHRNGLSVLYGKHLRHLLRISGSSKGTRPPKAQRTTPSSSEPMQDTPPALPSSFMDQQMLWPDMVQFSTMSDDQIAQVLNQPGNVFEPSSSFGGNMSWEEMNNFDWLNWPAFNAN

SEQ ID NO: 55 - DNA, 2678 bp, Trichoderma reesei
atggcctcaatgcatacctttccctccgtctccgagcccggggcggcgcagtcgccggccatgctggtcgatgcctcgaatcccagtgacggagccagtccctacgacacctcgtccatgtctcagcagccctcgccgcgacagcagccgcaccagcatctcctgggcgcgtcttcacaggcactgcagcagtcgcagcagcagcagcagaaccaaccacagaacgagccgtcacaacagcagcaatcacagacccagcagcagcaacaacagcagcagcagcaacagcagcgcagcgtcaagcggcccaggcccgtcaagtcctgcaccgagtgtcgcaaacgaaagcttcgctgcgatcgcctctgcccctgctcgcaatgccaaaagtccaaccgtccctgcagatacgctcccgaccaagactcggccaacctgtccgatggctcggacgctgagggagccgagccgggccgcccagccaagcgcaactactcgcacagcgcgctgcctgcgctggccgcctcgtacggcgatgctgccgctcccagaccggccaaggcgagcgatcccgccagcctcccgttgctcgaggagctgtccatacggatggagcgcctggagaagcagatcctcgcgcggagtccaagcagggctgagggcggcggcggcggcagcagcagcagcatcgtcgccggggcgccagagacgatacggggattgacggtcaaacggggggccacgaggacgcggtactttgggcagaacgactcccgggtgatgctaaacctggtaagttggccctgatgaaccattggaggtccgagtctcgatttgatccccccttccttgatccatcccctttgctgtggcagcaagagctgactctttctgttttctctctctcctagtttgacgatgccaaggcatatatagcccacaacttccggcgagagcccttccccagcttcgagaagctacataggcatctgcgaagcgaattagcaaagtctctgacccccatcaccgtctttgtcgactctatgatgcctatccacaagagaatgatggacatcttgcccagaaaggcagtctgtgaccgcctcatggcggcgtatttcgacacttcggagacgcaatacaggatcctccacaccccgacattttgggagcagtataaccagtactggcagggcaatccgcagcccgagcagtttcttccccagatgctgtcggtgttggcggtagcgtcgaggttcgacaccaaatccagggggctgggccaccaggagcgcgtggagggcgtgcacatcccgactgcgtgcgcgctcgttcggagctggctcgacagcctcaagggcaagcagctcgtggagattgccacgctgcaagtcgaagtgctgctgctgtttgcccagaggatgattatccagcggccgcaggattcgtggaaccatctgggcttcgtcgtgcgcatggccatgtccatgggccttcatagggacccctcggagtttgagccgcgcatgacgcccttccagggtgagatccgacggaggctgtggttcacggtcctcgacatggacctgtacatgtcgcttgcctccaacatgccctgtctcacccgagacggcgactattcctgccggccgccgcgcaacgtcgacgacagcgagctgttccccgacatgacggagctgccgccgtctcggccccttgaccaggcgaccgacagccagatacagatgtacaccatcatgacgcttcccacgcggatgaaggtggctcacatgatcaaccgcatcgataccatccgcgactaccaggaggtgctcgacgtgggcgggaagctcgaccggttcatggaggacatcaactacatcttcccgcggcatgcgtccctcagcgacgcgcagaagagcaggctctggcgcgaccgcgtcgtcctcgacatgcacgtgcggcgacctctgctggccctgtaccgcccctttgccatgggggtgcccgacgccccggcgcaaatctcgcgcgcgtatctgcggtcgtgcatggtgattctcaactacctggacgatttggaccactcgctgccgcactttcgagacgtgtccgagatgtacctgcagatgttgcggcgcgacgtcatccaggccgccctgagcgtctgctactttgtcaagccggccgtccgtcccgctgccgacagcgtcttgctcgcccagcacggcatacgggcctcgccaagccccgcggacgatttcccggcgtacgtcccggacgacctgatgctgtggtcgccgtcgcggctcattggcacggtgcagaagacgattgatctcctcgtgggcttcattcgcggcagcgacgtcaaggatatcctgtgcgtcgcggtcgtgctggagacggtgaggaggcccgacatccgaagcgacgaaatcaccaagaatctgtttggcctgctggacgcttgcctgagggcgagcagcatgaccctggacaagctccacgctttgtcactaggcggcggcggcggcggcagcagcagcagcaacgagtaccagcacgaggcgtatggccatggcaggatgccgtttgtgcagcacggctacggcatggggggccattcggagacgacggagttgggcggctggatcatgtgggatggctgggattga

SEQ ID NO: 56 - protein, 852 aa, Trichoderma reesei
MASMHTFPSVSEPGAAQSPAMLVDASNPSDGASPYDTSSMSQQPSPRQQPHQHLLGASSQALQQSQQQQQNQPQNEPSQQQQSQTQQQQQQQQQQQQRSVKRPRPVKSCTECRKRKLRCDRLCPCSQCQKSNRPCRYAPDQDSANLSDGSDAEGAEPGRPAKRNYSHSALPALAASYGDAAAPRPAKASDPASLPLLEELSIRMERLEKQILARSPSRAEGGGGGSSSSIVAGAPETIRGLTVKRGATRTRYFGQNDSRVMLNLFDDAKAYIAHNFRREPFPSFEKLHRHLRSELAKSLTPITVFVDSMMPIHKRMMDILPRKAVCDRLMAAYFDTSETQYRILHTPTFWEQYNQYWQGNPQPEQFLPQMLSVLAVASRFDTKSRGLGHQERVEGVHIPTACALVRSWLDSLKGKQLVEIATLQVEVLLLFAQRMIIQRPQDSWNHLGFVVRMAMSMGLHRDPSEFEPRMTPFQGEIRRRLWFTVLDMDLYMSLASNMPCLTRDGDYSCRPPRNVDDSELFPDMTELPPSRPLDQATDSQIQMYTIMTLPTRMKVAHMINRIDTIRDYQEVLDVGGKLDRFMEDINYIFPRHASLSDAQKSRLWRDRVVLDMHVRRPLLALYRPFAMGVPDAPAQISRAYLRSCMVILNYLDDLDHSLPHFRDVSEMYLQMLRRDVIQAALSVCYFVKPAVRPAADSVLLAQHGIRASPSPADDFPAYVPDDLMLWSPSRLIGTVQKTIDLLVGFIRGSDVKDILCVAVVLETVRRPDIRSDEITKNLFGLLDACLRASSMTLDKLHALSLGGGGGGSSSSNEYQHEAYGHGRMPFVQHGYGMGGHSETTELGGWIMWDGWD

SEQ ID NO: 57 - DNA, 838 bp, Acremonium alcalophilum
atgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaa

SEQ ID NO: 58 - protein, 227 aa, Acremonium alcalophilum
MKLLPSLIGLASLASLAVARIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 59 - DNA, 50 bp, Artificial Sequence
  Artificial DNA Sequence
gagtcgacctgcaggcatgcgtttaaacttggccacctacactgctacta

SEQ ID NO: 60 - DNA, 43 bp, Artificial Sequence
  Artificial DNA Primer
cgtgaagccgtttaaatgaaactagctccagatggaaatatac

SEQ ID NO: 61 - DNA, 42 bp, Artificial Sequence
  Artificial DNA Primer
tatttccatctggagctagtttcatttaaacggcttcacggg

SEQ ID NO: 62 - DNA, 44 bp, Artificial Sequence
  Artificial DNA Sequence
tcgttcgaaattttcttctagagagttcaaggaagaaacagtgc

SEQ ID NO: 63 - DNA, 44 bp, Artificial Sequence
  Artificial DNA Sequence
gaagaatcgactggctgcctactagctccagatggaaatatact

SEQ ID NO: 64 - DNA, 44 bp, Artificial Sequence
  Artificial DNA Primer
tgtttcttccttgaactctctagaagaaaatttcgaacgaaccg

SEQ ID NO: 65 - DNA, 42 bp, Artificial Sequence
  Artificial DNA Primer
tatttccatctggagctagtaggcagccagtcgattcttctt

SEQ ID NO: 66 - DNA, 48 bp, Artificial Sequence
  Artificial DNA Primer
gctatgaccatgattacgccgtttaaaccgtccagataatgcgcacgc

SEQ ID NO: 67 - DNA, 48 bp, Artificial Sequence
  Artificial DNA Primer
gagtcgacctgcaggcatgcgtttaaacacacacaggggtaccgtttc

SEQ ID NO: 68 - DNA, 43 bp, Artificial Sequence
  Artificial DNA Primer
cgtgaagccgtttaaatgaagttgacggttgagcagaaaacgc

SEQ ID NO: 69 - DNA, 43 bp, Artificial Sequence
  Artificial DNA Primer
ttttctgctcaaccgtcaacttcatttaaacggcttcacgggc

SEQ ID NO: 70 - DNA, 44 bp, Artificial Sequence
  Artificial DNA Primer
gagtggtgggtttggtttgcgagagttcaaggaagaaacagtgc

SEQ ID NO: 71 - DNA, 43 bp, Artificial Sequence
  Artificial DNA Primer
tgtttcttccttgaactctcgcaaaccaaacccaccactctac

SEQ ID NO: 72 - DNA, 42 bp, Artificial Sequence
  Artificial DNA Primer
gatcagttcggatacgcgctgttgacggttgagcagaaaacg

SEQ ID NO: 73 - DNA, 42 bp, Artificial Sequence
  Artificial DNA Primer
ttttctgctcaaccgtcaacagcgcgtatccgaactgatcta

SEQ ID NO: 74 - DNA, 53 bp, Artificial Sequence
  Artificial DNA Primer
gctatgaccatgattacgccgtttaaacctacctgtcgaagaaataaaagagg

SEQ ID NO: 75 - DNA, 20 bp, Artificial Sequence
  Artificial DNA Primer
tgaccgggcaggggatcgcc

SEQ ID NO: 76 - DNA, 24 bp, Artificial Sequence
  Artificial DNA Primer
ctggggcgtcaagggacctgaatg

SEQ ID NO: 77 - DNA, 25 bp, Artificial Sequence
  Artificial DNA Primer
ctacatcgaagctgaaagcacgaga

SEQ ID NO: 78 - DNA, 23 bp, Artificial Sequence
  Artificial DNA Primer
gggacgccctgctgcaacttacc

SEQ ID NO: 79 - DNA, 21 bp, Artificial Sequence
  Artificial DNA Primer
cgcccttcgacgagtcggcac

SEQ ID NO: 80 - DNA, 20 bp, Artificial Sequence
  Artificial DNA Primer
tgaccgggcaggggatcgcc

SEQ ID NO: 81 - DNA, 21 bp, Artificial Sequence
  Artificial DNA Primer
cgcccttcgacgagtcggcac

SEQ ID NO: 82 - DNA, 25 bp, Artificial Sequence
  Artificial DNA Primer
ctacatcgaagctgaaagcacgaga

SEQ ID NO: 83 - DNA, 22 bp, Artificial Sequence
  Artificial DNA Primer
tgccctggtttcgcgcatacgg

SEQ ID NO: 84 - DNA, 26 bp, Artificial Sequence
  Artificial DNA Primer
acggataggagcagcaaagcaaaggc

SEQ ID NO: 85 - DNA, 25 bp, Artificial Sequence
  Artificial DNA Primer
ctacatcgaagctgaaagcacgaga

SEQ ID NO: 86 - DNA, 23 bp, Artificial Sequence
  Artificial DNA Primer
gggacgccctgctgcaacttacc

SEQ ID NO: 87 - DNA, 25 bp, Artificial Sequence
  Artificial DNA Primer
gagacgagactggagtcgttgccgc

SEQ ID NO: 88 - DNA, 22 bp, Artificial Sequence
  Artificial DNA Primer
tgccctggtttcgcgcatacgg

SEQ ID NO: 89 - DNA, 25 bp, Artificial Sequence
  Artificial DNA Primer
gagacgagactggagtcgttgccgc

SEQ ID NO: 90 - DNA, 25 bp, Artificial Sequence
  Artificial DNA Primer
ctacatcgaagctgaaagcacgaga

SEQ ID NO: 91 - DNA, 22 bp, Artificial Sequence
  Artificial DNA Primer
tgccctggtttcgcgcatacgg

SEQ ID NO: 92 - DNA, 26 bp, Artificial Sequence
  Artificial DNA Primer
acggataggagcagcaaagcaaaggc

SEQ ID NO: 93 - DNA, 25 bp, Artificial Sequence
  Artificial DNA Primer
ctacatcgaagctgaaagcacgaga

SEQ ID NO: 94 - DNA, 23 bp, Artificial Sequence
  Artificial DNA Primer
gggacgccctgctgcaacttacc

SEQ ID NO: 95 - DNA, 25 bp, Artificial Sequence
  Artificial DNA Primer
gagacgagactggagtcgttgccgc

SEQ ID NO: 96 - DNA, 22 bp, Artificial Sequence
  Artificial DNA Primer
tgccctggtttcgcgcatacgg

SEQ ID NO: 97 - DNA, 25 bp, Artificial Sequence
  Artificial DNA Primer
gagacgagactggagtcgttgccgc

SEQ ID NO: 98 - DNA, 25 bp, Artificial Sequence
  Artificial DNA Primer
ctacatcgaagctgaaagcacgaga

SEQ ID NO: 99 - DNA, 8655 bp, Artificial Sequence
  ARTIFICIAL DNA SEQUENCE
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcagtcaaaagacctttttaatttctactcttgtagatctacaacacgtcctcgagggttttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 100 - DNA, 63 bp, Artificial Sequence
  ARTIFICIAL DNA PRIMER
tttaatttctactcttgtagatgcattgtcaaagcatcgcccatttttttggctcttgggttc

SEQ ID NO: 101 - DNA, 879 bp, Artificial Sequence
  ARTIFICIAL DNA SEQUENCE
agagtcgacctgcaggcatgaaccggagagtactatcaaccgagcgcctggtttttacctttcaatctcgaccctgtcggtgcaggtgtcggtcttgatcccaaccctgcggcttccgtgcccagtagcgaaaatgccactccagacttatccgcgtttggcggtactggcataccactgggagggtatgatctgggaatgacgggaatgaatcaaaggtcccatcggtgatgggtattgctcctttttatttttttttttattttttttctctctttgcgagcggttctggttgggcgaatatggtgtcttggaaaagggtggggggttcacgacttctatatgctctgtatgctgaactgtttgtgtaactgagttgtatatccctgctttactccgtactctgatccattactttctttgtctgtgtcgtctaatctcgttgccatactgacccgcttaccgaccaatcatgccactggaaattcctttatagttcattctaatgtcttcacaagtggcttgcttgtcaagcaatggcatcattggtctagtggtagaattcgtcgttgccatcgacgaggcccgtgttcgattcacggatgatgcaaatttctactcttgtagatatctgcccagtggcgttcgcctttttttttgagcgcttacaggccacatttttaggtaagatgtcagccatgttgatggggcattgcgagtagcggtcgttcgaaacaagatggagtctggtcaggcatggaagcaaaagtcatggcgagaatatgtatgtcctcctttgagatcggaatatcgtggtcacatgttcaagcgtaatggtactgtaggatccagaaaaaaacaagctttctgctcgaggcc

SEQ ID NO: 102 - DNA, 27 bp, Artificial Sequence
  ARTIFICIAL DNA PRIMER
gaattcaaaagcgccagtcactgcgag

SEQ ID NO: 103 - DNA, 25 bp, Artificial Sequence
  ARTIFICIAL DNA PRIMER
ctacatcgaagctgaaagcacgaga

SEQ ID NO: 104 - DNA, 28 bp, Artificial Sequence
  ARTIFICIAL DNA PRIMER
tcgtcgagtcgaagatgagagaggatgg

SEQ ID NO: 105 - DNA, 23 bp, Artificial Sequence
  ARTIFICIAL DNA PRIMER
gggacgccctgctgcaacttacc

SEQ ID NO: 106 - DNA, 27 bp, Artificial Sequence
  ARTIFICIAL DNA PRIMER
gaattcaaaagcgccagtcactgcgag

SEQ ID NO: 107 - DNA, 25 bp, Artificial Sequence
  ARTIFICIAL DNA PRIMER
gagacgagactggagtcgttgccgc

SEQ ID NO: 108 - DNA, 20 bp, Artificial Sequence
  ARTIFICIAL DNA PRIMER
tgaccgggcaggggatcgcc

SEQ ID NO: 109 - DNA, 25 bp, Artificial Sequence
  ARTIFICIAL DNA PRIMER
ctacatcgaagctgaaagcacgaga

SEQ ID NO: 110 - DNA, 24 bp, Artificial Sequence
  ARTIFICIAL DNA PRIMER
ctggggcgtcaagggacctgaatg

SEQ ID NO: 111 - DNA, 23 bp, Artificial Sequence
  ARTIFICIAL DNA PRIMER
gggacgccctgctgcaacttacc

SEQ ID NO: 112 - DNA, 20 bp, Artificial Sequence
  ARTIFICIAL DNA PRIMER
tgaccgggcaggggatcgcc

SEQ ID NO: 113 - DNA, 21 bp, Artificial Sequence
  ARTIFICIAL DNA PRIMER
cgcccttcgacgagtcggcac

SEQ ID NO: 114 - DNA, 27 bp, Artificial Sequence
  ARTIFICIAL DNA PRIMER
gaattcaaaagcgccagtcactgcgag

SEQ ID NO: 115 - DNA, 25 bp, Artificial Sequence
  ARTIFICIAL DNA PRIMER
ctacatcgaagctgaaagcacgaga

SEQ ID NO: 116 - DNA, 28 bp, Artificial Sequence
  ARTIFICIAL DNA PRIMER
tcgtcgagtcgaagatgagagaggatgg

SEQ ID NO: 117 - DNA, 20 bp, Artificial Sequence
  ARTIFICIAL DNA PRIMER
tgaccgggcaggggatcgcc

SEQ ID NO: 118 - DNA, 25 bp, Artificial Sequence
  ARTIFICIAL DNA PRIMER
ctacatcgaagctgaaagcacgaga

SEQ ID NO: 119 - DNA, 24 bp, Artificial Sequence
  ARTIFICIAL DNA PRIMER
ctggggcgtcaagggacctgaatg

SEQ ID NO: 120 - DNA, 23 bp, Artificial Sequence
  ARTIFICIAL DNA PRIMER
gggacgccctgctgcaacttacc

SEQ ID NO: 121 - DNA, 20 bp, Artificial Sequence
  ARTIFICIAL DNA PRIMER
tgaccgggcaggggatcgcc

SEQ ID NO: 122 - DNA, 21 bp, Artificial Sequence
  ARTIFICIAL DNA PRIMER
cgcccttcgacgagtcggcac

SEQ ID NO: 123 - DNA, 1409 bp, Trichoderma reesei
atgttccccgagttgaccagtggcacctggtcaccacccaatggcagcactactcagcctctttcgccgagggatgtggcttcttcccctggtaacaggcgcaggaggcctgtagacgaagcggatgaagaggaagcccatcgaccacgcaccgtgccgcgggtacatcgtgaccccatgcagcgcgtgtcccgttctcctcaaagcccttcctccacgtcatggagctcaggaggttggaccactaccccagcaaccgccgtctctccgacttcctcctttgccaaaccagcaccaatggaggtgcaggagcggtcacctgctgttcggttcgctctaccattcccgctcccgagccagcctggtgcgcaccactcggaggtttcgccaagcaaggtccaagagtggcccagacactatagccatgattatacccaccaccaccgcgctcagtaccacccggacagcgacagaagcgaatctcgagaggctaccagggcatatagcatggattacacccactggcacaaccaccatacctatcagtcgcaacccacaagcccccgtttgccttacgacccgacaagatactctgcgggcgtgtatcccgctccgcatcacatggagcctaacccatacggggagtcaggggccacgtccggtggcggcgccagaccacgtaggcggcgcggcaaccttcccaaggagacgacagatcagttgcgggcctggctcaacgcgcacctgcaccacccttatcctacagaggatgagaagcagcagctgatgcgcacaactggacttcagatgagtaagtggcctctacatcgtccaattacccgatttgtgcctcgcggctaaccgcccccctctctttagatcaaatctccaactggttcatcaacgcaagaagacgccaagtacccagcttgctcagagagcgaaatgccgagattatcgatccaaaccgaatgatgaccagccccaacaggagatccaggtcctcgtccatcagtgacggcgatctcagctcgagcgaatgggggcctgacgctcaaggccagggcgattgggcagccagacgccgaagccgcagcgtataaaccttcgttgctcgagtcgaccgcattgcctagcagaatcaccctggcacgagaagagccatttccctccaaaaaaacagcattccctaaaacgaaaatggccggcctcaatggcaaaagagtccgaagagcaacggaatatacttcaggaaaaagctaatgggggtgtataaccctccctttttacgactacggttaattgaatgacatcacgagatgttattcgatggctcttctacctttttttttttttttttttttttttcatctcttacttcttcaaatccagtcatggttggcctgtga

SEQ ID NO: 124 - protein, 275 aa, Trichoderma reesei
MFPELTSGTWSPPNGSTTQPLSPRDVASSPGNRRRRPVDEADEEEAHRPRTVPRVHRDPMQRVSRSPQSPSSTSWSSGGWTTTPATAVSPTSSFAKPAPMEVQERSPAVRFALPFPLPSQPGAHHSEVSPSKVQEWPRHYSHDYTHHHRAQYHPDSDRSESREATRAYSMDYTHWHNHHTYQSQPTSPRLPYDPTRYSAGVYPAPHHMEPNPYGESGATSGGGARPRRRRGNLPKETTDQLRAWLNAHLHHPYPTEDEKQQLMRTTGLQMIMVGL

SEQ ID NO: 125 - DNA, 51 bp, Artificial Sequence
  ARTIFICIAL DNA PRIMER
gagtcgacctgcaggcatgcttaattaacaattcctcgtgacagtttctgc

SEQ ID NO: 126 - DNA, 46 bp, Artificial Sequence
  ARTIFICIAL DNA PRIMER
cttgctcggtcctggcgtagacttatcacaaagttagccaaacagg

SEQ ID NO: 127 - DNA, 20 bp, Artificial Sequence
  ARTIFICIAL DNA PRIMER
tctacgccaggaccgagcaa

SEQ ID NO: 128 - DNA, 20 bp, Artificial Sequence
  ARTIFICIAL DNA PRIMER
tggaaacgcaaccctgaagg

SEQ ID NO: 129 - DNA, 43 bp, Artificial Sequence
  ARTIFICIAL DNA PRIMER
tcccttcagggttgcgtttccatgaactaccagcatacacgac

SEQ ID NO: 130 - DNA, 43 bp, Artificial Sequence
  ARTIFICIAL DNA PRIMER
acagctatgaccatgattacgcctccttgtttgatcctagccc

SEQ ID NO: 131 - DNA, 28 bp, Artificial Sequence
  ARTIFICIAL DNA PRIMER
atgcccagtcgcgaataatcactcagcc

SEQ ID NO: 132 - DNA, 25 bp, Artificial Sequence
  ARTIFICIAL DNA PRIMER
ggctgagtagtgctgccattgggtg

SEQ ID NO: 133 - DNA, 33 bp, Artificial Sequence
  ARTIFICIAL DNA PRIMER
ccataaggtggcgttgttacatctccctgagag

SEQ ID NO: 134 - DNA, 23 bp, Artificial Sequence
  ARTIFICIAL DNA PRIMER
ccgtcctcggtcaggagccttgg

SEQ ID NO: 135 - DNA, 35 bp, Artificial Sequence
  ARTIFICIAL DNA PRIMER
cttacttcttcaaatccagtcatggttggcctgtg

SEQ ID NO: 136 - DNA, 35 bp, Artificial Sequence; identical to SEQ ID NO: 135
  ARTIFICIAL DNA PRIMER